SEQUENCE LISTING (73 sequences)

SEQ ID NO: 1 - DNA, 702 bp, homo sapiens
atgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggccttcttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 2 - protein, 233 aa, homo sapiens [sequence omitted]

SEQ ID NO: 3 - DNA, 720 bp, homo sapiens
atggcgcacgctgggagaagtggttacgataaccgggagatagtgatgaagtacatccattataagctgtcgcagaggggctacgagtgggatgcgggagatgtgggcgccgcgcccccgggggccgcccccgcrccgggcwtcttctcctcscagcccgggcacacgccccatmcagccgcatcccgggacccggtcgccaggacctcgccrctrcagaccccggctgcccccggcgccgccgsggggcytgcgctcagcccggtgccacctgtggtccacctgaccctccgccaggccggcgacgacttctcccgccgctaccgccgcgacttcgccgagatgtccagscagctgcacctgacgcccttcaccgcgcggggaygctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcamcctgggtgcctatctgggccacaagtga

SEQ ID NO: 4 - protein, 239 aa, homo sapiens
  misc_feature (48)..(48): Xaa can be any naturally occurring amino acid
  misc_feature (59)..(59): Xaa can be any naturally occurring amino acid
  misc_feature (82)..(82): Xaa can be any naturally occurring amino acid
  misc_feature (84)..(84): Xaa can be any naturally occurring amino acid
  misc_feature (117)..(117): Xaa can be any naturally occurring amino acid
  misc_feature (129)..(129): Xaa can be any naturally occurring amino acid
  misc_feature (231)..(231): Xaa can be any naturally occurring amino acid
MAHAGRSGYDNREIVMKYIHYKLSQRGYEWDAGDVGAAPPGAAPAPGXFSSQPGHTPHXAASRDPVARTSPLQTPAAPGAAXGXALSPVPPVVHLTLRQAGDDFSRRYRRDFAEMSXQLHLTPFTARGXFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACIXLGAYLGHK

SEQ ID NO: 5 - DNA, 1350 bp, homo sapiens
atgtctgctgaagtcatccatcaggttgaagaagcacttgatacagatgagaaggagatgctgctcttcttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtgaatcagactcactcagggctccccctgcatcacatcaggaggatgttcatgggagattcatgcccttatctagcagggaagccaaagatgtttttcattcagaactatgtggtgtcagagggccagctggaggacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccgtccctgtacctgcagtgcctctcccagaaactgagacaagaaagaaaacgcccactcctggatcttcacattgaactcaatggctacatgtatgattggaacagcagagtttctgccaaggagaaatattatgtttggctgcagcacactctgagaaagaaacttatcctctcctacacaatccatcacactggc

SEQ ID NO: 6 - protein, 445 aa, homo sapiens
MSAEVIHQVEEALDTDEKEMLLFLCRDVAIDVVPPNVRDLLDILRERGKLSVGDLAELLYRVRRFDLLKRILKMDRKAVETHLLRNPHLVSDYRVLMAEIGEDLDKSDVSSLIFLMKDYMGRGKISKEKSFLDLVVELEKLNLVAPDQLDLLEKCLKNIHRIDLKTKIQKYKQSVQGAGTSYRNVLQAAIQKSLKDPSNNFRSIPEERYKMKSKPLGICLIIDCIGNETELLRDTFTSLGYEVQKFLHLSMHGISQILGQFACMPEHRDYDSFVCVLVSRGGSQSVYGVNQTHSGLPLHHIRRMFMGDSCPYLAGKPKMFFIQNYVVSEGQLEDSSLLEVDGPAMKNVEFKAQKRGLCTVHREADFFWSLCTADMSLLEQSHSSPSLYLQCLSQKLRQERKRPLLDLHIELNGYMYDWNSRVSAKEKYYVWLQHTLRKKLILSYT

SEQ ID NO: 7 - DNA, 1026 bp, homo sapiens
atggatatcttcagggaaatcgcatcttctatgaaaggagagaatgtattcatttctccaccgtcaatctcgtcagtattgacaatactgtattatggagctaatggatccactgctgaacagctatcaaaatatgtagaaaaggaggcggacaagaataaggatgatatctcattcaagtccatgaataaagtatatgggcgatattctgcagtgtttaaagattcctttttgagaaaaattggagataatttccaaactgttgacttcactgattgtcgcactgtagatgcgatcaacaagtgtgttgatatcttcactgaggggaaaattaatccactattggatgaaccattgtctccagatacctgtctcctagcaattagtgccgtatactttaaagcaaaatggttgatgccatttgaaaaggaatttaccagtgattatcccttttacgtatctccaacggaaatggtagatgtaagtatgatgtctatgtacggcgaggcatttaatcacgcatctgtaaaagaatcattcggcaacttttcaatcatagaactgccatatgttggagatactagtatggtggtaattcttccagacaatattgatggactagaatccatagaacaaaatctaacagatacaaattttaagaaatggtgtgactctatggatgctatgtttatcgatgtgcacattcccaagtttaaggtaacaggctcgtataatctggtggatgcgctagtaaagttgggactgacagaggtgttcggttcaactggagattatagcaatatgtgtaattcagatgtgagtgtcgacgctatgatccacaaaacgtatatagatgtcaatgaagagtatacagaagcagctgcagcaacttgtgcgctggtggcagactgtgcatcaacagttacaaatgagttctgtgcagatcatccgttcatctatgtgattaggcatgtcgatggcaaaattcttttcgttggtagatattgctctccaacaactaattaa

SEQ ID NO: 8 - protein, 341 aa, homo sapiens
MDIFREIASSMKGENVFISPPSISSVLTILYYGANGSTAEQLSKYVEKEADKNKDDISFKSMNKVYGRYSAVFKDSFLRKIGDNFQTVDFTDCRTVDAINKCVDIFTEGKINPLLDEPLSPDTCLLAISAVYFKAKWLMPFEKEFTSDYPFYVSPTEMVDVSMMSMYGEAFNHASVKESFGNFSIIELPYVGDTSMVVILPDNIDGLESIEQNLTDTNFKKWCDSMDAMFIDVHIPKFKVTGSYNLVDALVKLGLTEVFGSTGDYSNMCNSDVSVDAMIHKTYIDVNEEYTEAAAATCALVADCASTVTNEFCADHPFIYVIRHVDGKILFVGRYCSPTTN

SEQ ID NO: 9 - DNA, 1494 bp, homo sapiens
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgttttcgttccttgtggacatctagtcacttgtaaacaatgtgctgaagcagttgacaagtgtcccatgtgctacacagtcattactttcaagcaaaaaatttttatgtcttaa

SEQ ID NO: 10 - protein, 497 aa, homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 11 - DNA, 291 bp, homo sapiens
atgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaa

SEQ ID NO: 12 - protein, 96 aa, homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 13 - DNA, 435 bp, homo sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 14 - protein, 144 aa, homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 15 - DNA, 450 bp, homo sapiens
atggatgcaatgaagagagggctctgctgtgtgctgctgctgtgtggagcagtcttcgtttcgcccagccaggaaatccatgcccgattcagaagaggcgcccgcaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 16 - protein, 149 aa, homo sapiens
MDAMKRGLCCVLLLCGAVFVSPSQEIHARFRRGARNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 17 - DNA, 7173 bp, artificial
  artificial sequence
gtatctactaatcagatctattagagatattattaattctggtgcaatatgacaaaaattatacactaattagcgtctcgtttcagacatggatctgtcacgaattaatacttggaagtctaagcagctgaaaagctttctctctagcaaagatgcatttaaggcggatgtccatggacatagtgccttgtattatgcaatagctgataataacgtgcgtctagtatgtacgttgttgaacgctggagcattgaaaaatcttctagagaatgaatttccattacatcaggcagccacattggaagataccaaaatagtaaagattttgctattcagtggactggatgattcgaggtacccctggcgaaagggggatgtgctgcaaggcgattaagttgggtaacgccagggttttcccagtcacgacgttgtaaaacgacggccagtgccaagcttaaggtgcacggcccacgtggccactagtacttctcgaggtcgacggtatcgataagcttgatatcgaattcctgcagcccgggggatccataacttcgtataatgtatgctatacgaagttatctaggtagaaaaatcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagctttccgtatgtagcatcaccttcaccctctccactgacagaaaatttgtgcccattaacatcaccatctaattcaacaagaattgggacaactccagtgaaaagttcttctcctttgctagccattttttctaccgccattcgcgaaaccgcggaaacgcgtaagccggctatttatgattatttctcgctttcaatttaacacaaccctcaagaacctttgtatttattttcaatttttaggctagataacttcgtataatgtatgctatacgaagttatgcggccgccatatgcatcctaggcctattaatattccggagtatacatcgatcgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaggggcaactagtaagatgggagatctggcgcgcctgcagagaattcgtttatctgcagaattcggcttgggccctactcgagaattaattaaaaagcaaacttaagcttggtaccgagctcggatctcactcctggactggctcccagcagtcaaaggggatgacaagcagaaagtccttcaggttctctttgaaactttcaaaggtgataatctgggttgcacaggaagtttccggggttggagggcagtgctgcttgtagtggctggccatcatggtcaaggggcccttgagcttggtgaggctgccccgcaggccctgcttgtacagctccaggcgggtctgtaggcaggtcggctcctggaggtcaaacatttctgagatgacttctactgtttcattcatctcagcagcagtgtctctactcaggttcaggagacgccgggcctcctggatggcattcacatgctcccagggctgcgtgctggggctgggcgagcgggcgggtgcagagatgctgcaggccacagtgcccaagagcagcaggctctgcagccacatggtgaattcgatatcaagcttatcgataccgtcgacgacggtgactgcagaaaagacccatggaaaggaacagtctgttagtctgtcagctattatgtctggtggcgcgcgcggcagcaacgagtatccagcacagtggcggccgctcgagtctagagggcccgtttgcttatttatgattatttctcgctttcaatttaacacaaccctcaagaacctttgtatttattttcaatttttaggcctaaaaattgaaaataaatacaaaggttcttgagggttgtgttaaattgaaagcgagaaataatcataaatagccggcttacgcgtttccgcggtttcgcgattgagctcggatccactagtaacggccgccagtgtgctggaattctgcagatatccatcacactggcggccgctcgaggccaccatgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggctttgatatcttagggcgaattctgcagatgtagcggccgctagcatcgggggatcctctagagggccctattctatagtgtcacctaaatgctagagctctacgtagcggccgctagccatcgggggatcctctagagtcatcaacaatgaacctaaagtactagaaatggtatatgatgctacaattttacccgaaggtagtagcatggattgtataaacagacacatcaatatgtgtatacaacgcacctatagttctagtataattgccatattggatagattcctaatgatgaacaaggatgaactaaataatacacagtgtcatataattaaagaatttatgacatacgaacaaatggcgattgaccattatggagaatatgtaaacgctattctatatcaaattcgtaaaagacctaatcaacatcacaccattaatctgtttaaaaaaataaaaagaacccggtatgacacttttaaagtggatcccgtagaattcgtaaaaaaagttatcggatttgtatctatcttgaacaaatataaaccggtttatagttacgtcctgtacgagaacgtcctgtacgatgagttcaaatgtttcattgactacgtggaaactaagtatttctaaaattaatgatgcattaatttttgtattgattctcaatcctaaaaactaaaatatgaataagtattaaacatagcggtgtactaattgatttaacataaaaaatagttgttaactaatcatgaggactctacttattagatatattctttggagaaatgacaacgatcaaaccgggcatgcaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactatagaatacgaattccctcctgaaaaactggaatttaatacaccatttgtgttcatcatcagacatgatattactggatttatattgtttatgggtaaggtagaatctccttaatatgggtacggtgtaaggaatcattattttatttatattgatgggtacgtgaaatctgaattttcttaataaatattatttttattaaatgtgtatatgttgttttgcgatagccat

SEQ ID NO: 18 - DNA, 1500 bp, artificial
  artificial sequence
atgggcgccagagccagcgtgctgagcggcggcaagctggacgcctgggagaagatcagactgaggcctggcggcaagaagaagtaccggctgaagcacctggtgtgggccagcagagagctggagagattcgccctgaaccctagcctgctggagaccgccgagggctgccagcagatcatggagcagctgcagcctgccctgaaaaccggcaccgaggagctgagaagcctgtacaacaccgtggccaccctgtactgcgtgcaccagcggatcgacgtgaaggataccaaggaggccctggacaagatcgaggagatccagaacaagagcaagcagaaaacccagcaggccgctgccgacaccggcaatagcagcaaagtgagccagaactaccccatcgtgcagaacgcccagggccagatggtgcaccagagcctgagccccagaaccctgaatgcctgggtgaaagtgattgaggagaaggccttcagccccgaagtgatccctatgttcagcgccctgagcgagggcgccaccccccaggatctgaacatgatgctgaacatcgtgggcggccaccaggccgccatgcagatgctgaaggacaccatcaatgaggaggccgccgagtgggacagactgcaccccgtgcacgccggacccatcccccctggccagatgagagagcccagaggcagcgacatcgccggcaccacaagcacccctcaggagcagatcggctggatgaccagcaacccccccatccccgtgggcgacatctacaagcggtggatcatcctgggcctgaacaagatcgtgcggatgtacagccctgtgagcatcctggacatcaagcagggccccaaggagcccttcagagactacgtggaccggttcttcaagaccctgagagccgagcaggccacccaggaagtgaagaactggatgaccgagaccctgctggtgcagaatgccaaccccgactgcaagagcatcctgagagccctgggccctggcgccaccctggaggagatgatgaccgcctgccagggcgtgggcggacctggccacaaggccagagtgctggccgaggccatgagccaagtgcagcacaccaacatcatgatgcagcggggcaacttcagaggccagaagcggatcaagtgcttcaactgcggcaaggagggccacctggccagaaactgcagagcccccaggaagaagggctgctggaagtgtggaaaggaaggccaccagatgaaggactgcaccgagaggcaggccaatttcctgggcaagatctggcctagcagcaagggcagacccggcaatttcccccagagcagacccgagcccaccgcccctcccgccgagatcttcggcatgggcgaggagatcaccagccctcctaagcaggagcagaaggacagagagcagaaccctcctagcgtgagcctgaagagcctgttcggcaacgatcccctgagccagaagtga

SEQ ID NO: 19 - protein, 499 aa, artificial
  artificial sequence
MGARASVLSGGKLDAWEKIRLRPGGKKKYRLKHLVWASRELERFALNPSLLETAEGCQQIMEQLQPALKTGTEELRSLYNTVATLYCVHQRIDVKDTKEALDKIEEIQNKSKQKTQQAAADTGNSSKVSQNYPIVQNAQGQMVHQSLSPRTLNAWVKVIEEKAFSPEVIPMFSALSEGATPQDLNMMLNIVGGHQAAMQMLKDTINEEAAEWDRLHPVHAGPIPPGQMREPRGSDIAGTTSTPQEQIGWMTSNPPIPVGDIYKRWIILGLNKIVRMYSPVSILDIKQGPKEPFRDYVDRFFKTLRAEQATQEVKNWMTETLLVQNANPDCKSILRALGPGATLEEMMTACQGVGGPGHKARVLAEAMSQVQHTNIMMQRGNFRGQKRIKCFNCGKEGHLARNCRAPRKKGCWKCGKEGHQMKDCTERQANFLGKIWPSSKGRPGNFPQSRPEPTAPPAEIFGMGEEITSPPKQEQKDREQNPPSVSLKSLFGNDPLSQK

SEQ ID NO: 20 - DNA, 3014 bp, artificial
  artificial sequence
atgttcttcagggagaacctggccttccagcagggcgaggccagaaagttcagcagcgagcagaccagagccaatagccccacctccagagatctgtgggacggcggcagagacagcctgcccagcgaggccggagccgagagacagggcaccggccccaccttcagcttccctcagatcaccctgtggcagagacccctggtgaccgtgaagatcggcggccagctgaaggaggctctgctggatacaggcgccgatgataccgtgctggaggacatcaacctgcccggcaagtggaagcctaagatgatcggcggcatcgggggcttcatcaaagtgaagcagtacgaccagatcctgatcgagatctgcggcaagaaggccatcggcaccgtgctggtcggccccacccctgtgaatatcatcggccggaacatgctgacccagatcggctgcaccctgaacttccccatcagccccatcgagaccgtgcctgtgaagctgaagcctggcatggacggccccaaagtgaaacagtggcccctgaccgaggagaagatcaaggccctgacagagatctgcaccgagatggagaaggagggcaagatcagcaagatcggccccgagaacccctacaacacccccatcttcgccatcaagaagaaggacagcaccaagtggcggaaactggtggacttccgggagctgaacaagaggacccaggacttctgggaagtgcagctgggcatcccccaccctgccggcctgaagaagaagaagagcgtgacagtgctggacgtgggcgatgcctacttcagcgtgcccctggacgagagcttcaggaagtacaccgccttcaccatccccagcaccaacaacgagacccccggcatcagataccagtacaacgtgctgcctcagggctggaagggcagccccgccatcttccagagcagcatgaccaagatcctggagcccttcaggagcaagaaccccgagatcatcatctaccagtacatgaacgacctgtacgtgggcagcgacctggagatcggccagcacagagccaagatcgaggagctgagagcccacctgctgagctggggcttcaccacccccgataagaagcaccagaaggagccccctttcctgtggatgggctacgagctgcaccccgataagtggaccgtgcagcccatcaagctgcctgagaaggagagctggaccgtgaacgacatccagaaactggtgggcaagctgaattgggccagccagatctacgccgggatcaaagtgaaacagctgtgcaagctgctgaggggcgccaaagccctgaccgatatcgtgaccctgaccgaagaggccgagctggagctggccgagaacagggagatcctgaaggatcctgtgcacggcgtgtactacgaccccagcaaggatctgatcgccgagatccagaagcagggccaggatcagtggacctaccagatctaccaggagcctttcaagaacctgaaaaccggcaagtacgccaggaagagaagcgcccacaccaacgacgtgaagcagctggccgaagtggtgcagaaagtggtgatggagagcatcgtgatctggggaaagacccccaagttcaagctgcccatccagaaggagacatgggagacctggtggatggattactggcaggccacctggatccccgagtgggagttcgtgaacacccccccactggtgaagctgtggtatcagctggagaaggaccccatcgctggcgccgagaccttctacgtggacggagccgccaatagagagaccaagctgggcaaggccggctacgtgaccgacagaggcagacagaaagtggtgtccctgaccgagaccaccaaccagaaaaccgagctgcacgccatccatctggccctgcaggacagcggcagcgaagtgaacatcgtgaccgactcccagtacgccctgggcatcatccaggcccagcccgacagaagcgagagcgagctggtgaaccagatcatcgagaagctgatcgagaaggacaaagtgtacctgagctgggtgcccgcccacaagggcatcggcggcaacgagcaagtggacagctggtgagcagcggcatccggaaagtgctgttcctggacggcatcgataaggcccaggaggagcacgagagataccactccaactggagggccatggccagcgacttcaacctgcctcccatcgtggccaaggagatcgtggccagctgcgataagtgtcagctgaagggggaggccatgcacggccaagtggactgcagccctggcatctggcagctggattgcacccacctggagggcaaagtgatcctggtggccgtgcacgtggccagcggctacatcgaggccgaagtgatccccgccgagaccggccaggagaccgcctacttcctgctgaagctggccggcagatggcccgtgaaagtggtgcacaccgacaacggcagcaatttcaccagcgccgctgtgaaggccgcctgttggtgggccaacgtgcagcaggagttcggcatcccctacaaccctcagagccagggcgtggtggagagcatgaacaaggagctgaagaagatcatcggccaagtgagagagcaggccgagcacctgaaaacagccgtgcagatggctgtgttcatccacaacttcaagcggaagggcggcattggcggctacagcgccggagagcggatcatcgacatcatcgccaccgatatccagaccaaggaactgcagaagcagatcacaaagatccagaacttcagagtgtactaccgggacagcagggaccccatctggaagggccctgccaagctgctgtggaagggcgagggcgccgtggtgatccaggacaacagcgacatcaaagtggtgccccggaggaaggccaagatcatccgggactacggcaagcagatggccggcgacgactgcgtggccggcaggcaggatgaggattga

SEQ ID NO: 21 - protein, 1004 aa, artificial
  artificial consensus sequence
MFFRENLAFQQGEARKFSSEQTRANSPTSRDLWDGGRDSLPSEAGAERQGTGPTFSFPQITLWQRPLVTVKIGGQLKEALLDTGADDTVLEDINLPGKWKPKMIGGIGGFIKVKQYDQILIEICGKKAIGTVLVGPTPVNIIGRNMLTQIGCTLNFPISPIETVPVKLKPGMDGPKVKQWPLTEEKIKALTEICTEMEKEGKISKIGPENPYNTPIFAIKKKDSTKWRKLVDFRELNKRTQDFWEVQLGIPHPAGLKKKKSVTVLDVGDAYFSVPLDESFRKYTAFTIPSTNNETPGIRYQYNVLPQGWKGSPAIFQSSMTKILEPFRSKNPEIIIYQYMNDLYVGSDLEIGQHRAKIEELRAHLLSWGFTTPDKKHQKEPPFLWMGYELHPDKWTVQPIKLPEKESWTVNDIQKLVGKLNWASQIYAGIKVKQLCKLLRGAKALTDIVTLTEEAELELAENREILKDPVHGVYYDPSKDLIAEIQKQGQDQWTYQIYQEPFKNLKTGKYARKRSAHTNDVKQLAEVVQKVVMESIVIWGKTPKFKLPIQKETWETWWMDYWQATWIPEWEFVNTPPLVKLWYQLEKDPIAGAETFYVDGAANRETKLGKAGYVTDRGRQKVVSLTETTNQKTELHAIHLALQDSGSEVNIVTDSQYALGIIQAQPDRSESELVNQIIEKLIEKDKVYLSWVPAHKGIGGNEQVDKLVSSGIRKVLFLDGIDKAQEEHERYHSNWRAMASDFNLPPIVAKEIVASCDKCQLKGEAMHGQVDCSPGIWQLDCTHLEGKVILVAVHVASGYIEAEVIPAETGQETAYFLLKLAGRWPVKVVHTDNGSNFTSAAVKAACWWANVQQEFGIPYNPQSQGVVESMNKELKKIIGQVREQAEHLKTAVQMAVFIHNFKRKGGIGGYSAGERIIDIIATDIQTKELQKQITKIQNFRVYYRDSRDPIWKGPAKLLWKGEGAVVIQDNSDIKVVPRRKAKIIRDYGKQMAGDDCVAGRQDED

SEQ ID NO: 22 - DNA, 2400 bp, artificial
  artificial consensus sequence
atgcgcgtgatgggcatccagaggaactgccagcacctgtggagatggggcaccatgatcctgggcatgatcatcatctgctctgccgccgagaacctgtgggtgaccgtgtactacggcgtgcccgtgtggaaggacgccgagaccaccctgttctgcgccagcgacgccaaggcctacgataccgaagtgcacaacgtgtgggccacccacgcctgcgtgcctaccgatcccaacccccaggagatcaacctggagaacgtgaccgaggagttcaacatgtggaagaacaacatggtggagcagatgcacaccgacatcatcagcctgtgggaccagagcctgaagccttgcgtgaagctgacccctctgtgcgtgaccctgaactgcagcaacgccgccaactgcaataccagcgccatcacccaggcctgtcccaaagtgagcttcgagcccatccccatccactactgcgcccctgccggcttcgccatcctgaagtgcaaggacaaggagtttaacggcaccggcccctgcaagaacgtgagcaccgtgcagtgcacccacggcatcaagcccgtggtgagcacccagctgctgctgaacggcagcctggccgaggaagaagtgatgatccggagcgagaacatcaccaacaacgccaagaacatcatcgtgcagctgaccaagcccgtgaagatcaactgcacccggcccaacaacaacacccggaagagcatcagaatcggccctggccaggccttctacgccaccggcgacatcatcggcgatatcaggcaggcccactgcaatgtgagccggaccgagtggaacgagaccctgcagaaagtggccaagcagctgcggaagtacttcaacaacaagaccatcatcttcaccaacagcagcggcggagatctggagatcaccacccacagcttcaattgtggcggcgagttcttctactgcaacacctccggcctgttcaacagcacctggaacggcaacggcaccaagaagaagaacagcaccgagagcaacgacaccatcaccctgccctgccggatcaagcagatcatcaatatgtggcagcgcgtgggccaggccatgtacgcccctcccatccagggcgtgatcagatgcgagagcaacatcaccggcctgctgctgaccagagatggcggcgacaacaacagcaagaacgagaccttcagacctggcggcggagacatgagggacaactggcggagcgagctgtacaagtacaaagtggtgaagatcgagcccctgggcgtggcccccaccaaggccaagagaagagtggtggagcgggagaagagagccgtgggcatcggcgccgtgttcctgggcttcctgggagccgccggaagcaccatgggagccgccagcatcaccctgaccgtgcaggccagacagctgctgagcggcattgtgcagcagcagagcaacctgctgagagccatcgaggcccagcagcacctgctgaagctgacagtgtggggcattaagcagctgcaggcccgcgtgctggccgtggagagatacctgaaggaccagcagctgctgggcatctggggctgcagcggcaagctgatctgcaccaccaacgtgccctggaatagcagctggagcaacaagagccagagcgagatctgggacaacatgacctggctgcagtgggacaaggagatcagcaactacaccgatatcatctacaacctgatcgaggagagccagaaccagcaggagaagaacgagcaggatctgctggccctggacaagtgggccaacctgtggaactggttcgacatcagcaactggctgtggtacatcaagatcttcatcatgatcgtgggcggcctgatcggcctgagaatcgtgttcgccgtgctgagcgtgatcaacagagtgcggcagggctacagccccctgagcttccagacccacacccccaaccctggcggcctggacagacccggcagaatcgaggaggagggcggcgagcagggcagagacaggagcatcagactggtgagcggcttcctggccctggcctgggacgacctgagaagcctgtgcctgttcagctaccaccggctgagggacttcatcctgatcgccgccagaaccgtggagctgctgggacacagctccctgaagggcctgagactgggctgggagggcctgaagtacctgtggaatctgctgctgtactggggcagggagctgaagatcagcgccattaacctgctggacaccatcgccatcgccgtggccggctggaccgacagagtgatcgagatcggccagaggatctgcagagccattctgaacatcccccggaggatcagacagggcctggagcgggccctgctgtga

SEQ ID NO: 23 - protein, 799 aa, artificial
  artificial consensus sequence
MRVMGIQRNCQHLWRWGTMILGMIIICSAAENLWVTVYYGVPVWKDAETTLFCASDAKAYDTEVHNVWATHACVPTDPNPQEINLENVTEEFNMWKNNMVEQMHTDIISLWDQSLKPCVKLTPLCVTLNCSNAANCNTSAITQACPKVSFEPIPIHYCAPAGFAILKCKDKEFNGTGPCKNVSTVQCTHGIKPVVSTQLLLNGSLAEEEVMIRSENITNNAKNIIVQLTKPVKINCTRPNNNTRKSIRIGPGQAFYATGDIIGDIRQAHCNVSRTEWNETLQKVAKQLRKYFNNKTIIFTNSSGGDLEITTHSFNCGGEFFYCNTSGLFNSTWNGNGTKKKNSTESNDTITLPCRIKQIINMWQRVGQAMYAPPIQGVIRCESNITGLLLTRDGGDNNSKNETFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQSEIWDNMTWLQWDKEISNYTDIIYNLIEESQNQQEKNEQDLLALDKWANLWNWFDISNWLWYIKIFIMIVGGLIGLRIVFAVLSVINRVRQGYSPLSFQTHTPNPGGLDRPGRIEEEGGEQGRDRSIRLVSGFLALAWDDLRSLCLFSYHRLRDFILIAARTVELLGHSSLKGLRLGWEGLKYLWNLLLYWGRELKISAINLLDTIAIAVAGWTDRVIEIGQRICRAILNIPRRIRQGLERALL

SEQ ID NO: 24 - DNA, 609 bp, artificial
  artificial consensus sequence
atgaagtggagcaagagcagcatcgtgggctggcctgaagtgcgggagcggatcagaagaaccccccctgccgccaagggcgtgggcgccgtgagccaggacctggacaagcacggagccgtgaccagcagcaacatcaaccaccctagctgcgcctggctggaggcccaggaggaggaggaagtgggcttccctgtgagaccccaagtgcccctgagacccatgacctacaagggcgccttcgacctgagccacttcctgaaggagaagggcggcctggacggcctgatctacagcaagaagcggcaggagatcctggatctgtgggtgtaccacacccagggctacttccccgactggcagaattacacccctggccctggcatcagataccctctgaccttcggctggtgcttcaagctggtgcccgtggaccccgacgaagtggaggaggccaccgagggcgagaacaatagcctgctgcaccccatctgccagcacggcatggacgatgaggagcgggaagtgctgatgtggaagttcgacagcaggctggccctgaagcacagagccagagagctgcaccccgagttctacaaggactgctga

SEQ ID NO: 25 - protein, 202 aa, artificial
  artificial consensus sequence
MKWSKSSIVGWPEVRERIRRTPPAAKGVGAVSQDLDKHGAVTSSNINHPSCAWLEAQEEEEVGFPVRPQVPLRPMTYKGAFDLSHFLKEKGGLDGLIYSKKRQEILDLWVYHTQGYFPDWQNYTPGPGIRYPLTFGWCFKLVPVDPDEVEEATEGENNSLLHPICQHGMDDEEREVLMWKFDSRLALKHRARELHPEFYKDC

SEQ ID NO: 26 - DNA, 1503 bp, artificial
  artificial consensus sequence
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgaggtgaaggacaccaaggaggccctggagaagatcgaggaggagcagaacaagagcaagaagaaggcccagcaggccgccgccgacaccggcaacagcagccaagtgagccagaactaccccatcgtgcagaacctgcagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcctgcaccccgtgcacgccggccccatcgcccccggccagatgcgcgagccccgcggcagcgacatcgccggcaccacgagcaccctgcaggagcagatcggctggatgaccaacaacccccctatccccgtgggcgagatctacaagcgctggatcatcctgggcctgaacaagatcgtgcgcatgtacagccccacgagcatcctggacatccgccagggccccaaggagcccttccgcgactacgtggaccgcttctacaagaccctgcgggccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagaccgtgaagtgcttcaactgcgggaaggagggccacatcgccaagaactgccgcgccccccgcaagaagggctgctggaagtgcggcaaggaggggcaccagatgaaggactgcaccgagcgccaggccaacttcctgggcaagatctggcccagccacaagggccgccccggcaacttcctgcagagccgccccgagcccaccgcccctcccgaggagagcttccgcttcggcgaggagaccaccacccccagccagaagcaggagcccatcgacaaggagctgtaccccctggccagcctgcgcagcctgttcggcaacgaccccagcagccagtaa

SEQ ID NO: 27 - protein, 500 aa, artificial
  artificial consensus sequence
MGARASVLSGGELDRWEKIRLRPGGKKKYKLKHIVWASRELERFAVNPGLLETSEGCRQILGQLQPSLQTGSEELRSLYNTVATLYCVHQRIEVKDTKEALEKIEEEQNKSKKKAQQAAADTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGNDPSSQ

SEQ ID NO: 28 - DNA, 3018 bp, artificial
  artificial consensus sequence
atggccttcttccgcgaggacctggccttcccccaaggcaaggcccgcgagttcagcagcgagcagacccgcgccaacagccccacccgccgcgagctgcaggtgtggggccgcgacaacaacagcctgagcgaggccggcgccgaccgccagggcaccgtgagcttcagcttcccccaaatcaccctgtggcagcgccccctggtgaccatcaagatcggcggccagctgaaggaggccctgctggacaccggcgccgacgacaccgtgctggaagagatgaacctgcccggccgctggaagcccaagatgatcggcggcatcggcggcttcatcaaagtgcgccagtacgaccagatcctgatcgagatctgcggccacaaggccatcggcaccgtgctcgtgggccccacccccgtgaacatcatcggccgcaacctgctgacccagatcggctgcaccctgaacttccccatcagccccatcgagaccgtgcccgtgaagctgaagcccggcatggacggccccaaggtgaagcagtggcccctgaccgaggagaagatcaaggccctggtggagatctgcaccgagatggagaaggagggcaagatcagcaagatcggccccgagaacccctacaacacccccgtgttcgccatcaagaagaaggacagcaccaagtggcgcaagctcgtggacttccgcgagctgaacaagcgcacccaggacttctgggaggtgcagctgggcatcccccaccccgccggcctgaagaagaagaagagcgtgaccgtgctggacgtgggcgacgcctacttcagcgtgcccctggacaaggacttccgcaagtacaccgccttcaccatccccagcatcaacaacgagacccccggcatccgctaccagtacaacgtgctgccccagggctggaagggcagccccgccatcttccagagcagcatgaccaagatcctggagcccttccgcaagcagaaccccgacatcgtgatctaccagtacatgaacgacctgtacgtgggcagcgacctggagatcggccagcaccgcaccaagatcgaggagctgcgccagcacctgctgcgctggggcttcaccacccccgacaagaagcaccagaaggagccccccttcctgtggatgggctacgagctgcaccccgacaagtggaccgtgcagcccatcgtgctgcccgagaaggacagctggaccgtgaacgacatccagaagctcgtgggcaagctgaactgggccagccagatctacgccggcatcaaggtgaagcagctgtgcaagctgctgcgcggcaccaaggccctgaccgaggtgatccccctgaccgaggaggccgagctggagctggccgagaaccgcgagatcctgaaggagcccgtgcacggcgtgtactacgaccccagcaaggacctgatcgccgagatccagaagcagggccagggccagtggacctaccagatctaccaggagcccttcaagaacctcaagaccggcaagtacgcccgcatgcgcggcgcccacaccaacgacgtgaagcagctgaccgaggccgtgcagaagatcgccaccgagagcatcgtgatctggggcaagacccccaagttcaagctgcccatccagaaggagacctgggagacctggtggaccgagtactggcaggccacctggatccccgagtgggagttcgtgaacacccctcccctggtgaagctgtggtatcagctggagaaggagcccatcgtgggcgccgagaccttctacgtggacggcgccgccaaccgcgagaccaagctgggcaaggccggctacgtgaccgaccgcggccgccagaaggtggtgagcctgaccgacaccaccaaccaaaagaccgagctgcaggccatccacctggccctgcaggacagcggcctggaggtgaacatcgtgaccgacagccagtacgccctgggcatcatccaggcccagcccgacaagagcgagagcgagctggtgagccagatcatcgagcagctgatcaagaaggagaaggtgtacctggcctgggtgcccgcccacaagggcatcggcggcaacgagcaggtggacaagctggtgagcgccggcatccgcaaggtgctgttcctggacggcatcgacaaggcccaggaggagcacgagaagtaccacagcaactggcgggccatggccagcgacttcaacctgccccccgtggtggccaaggagatcgtggccagctgcgacaagtgccagctgaagggcgaggccatgcacggccaggtggactgcagccccggcatctggcagctggactgcacccacctggagggcaagatcatcctggtggccgtgcacgtggccagcggctacatcgaggccgaggtgatccccgccgagaccggccaggagaccgcctacttcctgctgaagctggccggccgctggcccgtcaagaccatccacaccgacaacggcagcaacttcaccagcaccaccgtgaaggccgcctgttggtgggccggcatcaagcaggagttcggcatcccctacaacccccagagccagggcgtggtggagagcatgaacaaggagctgaagaagatcatcggccaagtgcgcgaccaggccgagcacctcaagaccgccgtgcagatggccgtgttcatccacaacttcaagcgcaagggcgggatcggcggctacagcgccggcgagcgcatcgtggacatcatcgccaccgacatccagaccaaggagctgcagaagcagatcaccaagatccagaacttccgcgtgtactaccgcgacagccgcgaccccctgtggaagggccccgccaagctgctgtggaagggcgagggcgccgtggtgatccaggacaacagcgacatcaaggtggtgccccgccgcaaggccaagatcatccgcgactacggcaagcagatggccggcgacgactgcgtggccagccgccaggacgaggactaa

SEQ ID NO: 29 - protein, 1005 aa, artificial
  artificial consensus sequence
MAFFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTKILEPFRKQNPDIVIYQYMNDLYVGSDLEIGQHRTKIEELRQHLLRWGFTTPDKKHQKEPPFLWMGYELHPDKWTVQPIVLPEKDSWTVNDIQKLVGKLNWASQIYAGIKVKQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWETWWTEYWQATWIPEWEFVNTPPLVKLWYQLEKEPIVGAETFYVDGAANRETKLGKAGYVTDRGRQKVVSLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKIILVAVHVASGYIEAEVIPAETGQETAYFLLKLAGRWPVKTIHTDNGSNFTSTTVKAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRDPLWKGPAKLLWKGEGAVVIQDNSDIKVVPRRKAKIIRDYGKQMAGDDCVASRQDED

SEQ ID NO: 30 - DNA, 2532 bp, artificial
  artificial consensus sequence
atgcgcgtgaagggcatccgcaagaactaccagcacctgtggcgctggggcaccatgctgctgggcatgctgatgatctgcagcgccgccgagcagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggaggtggtgctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccgacctgcgcaacgccaccaacaccacctccagcagctgggagaccatggagaagggcgagatcaagaactgcagcttcaacatcaccacctccatccgcgacaaggtgcagaaggagtacgccctgttctacaacctggacgtggtgcccatcgacaacgccagctaccgcctgatcagctgcaacaccagcgtgatcacccaggcctgccccaaagtgagcttcgagcccatccccatccactactgcgcccccgccggcttcgccatcctgaagtgcaacgacaagaagttcaacggcaccggcccctgcaccaacgtgagcaccgtgcagtgcacccacggcatccgccccgtggtgagcacccagctgctgctgaacggcagcctggccgaggaggaggtggtgatccgcagcgagaacttcaccgacaacgccaagaccatcatcgtgcagctgaacgagagcgtggagatcaactgcacccgccccaacaacaacacccgcaagagcatcaacatcggccccggccgcgccctgtacaccaccggcgagatcatcggcgacatccgccaggcccactgcaacatcagccgcgccaagtggaacaacaccctgaagcagatcgtgatcaagctgcgcgagcagttcggcaacaagaccatcgtgttcaaccagagcagcggcggcgaccccgagatcgtgatgcacagcttcaactgcggcggcgagttcttctactgcaacagcacccagctgttcacctggaacgacacccgcaagctgaacaacaccggccgcaacatcaccctgccctgccgcatcaagcagatcatcaacatgtggcaggaagtgggcaaggccatgtacgcccctcccatccgcggccagatccgctgcagcagcaacatcaccggcctgctgctgacccgcgacggcggcaaggacaccaacggcaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgggccgtgggcatcggcgccatgttcctgggcttcctgggcgccgccggcagcaccatgggcgccgccagcatgaccctgaccgtgcaggcccgccagctgctgagcggcatcgtgcagcagcagaacaacctgctgcgggccatcgaggcccagcagcacctgctgcagctgaccgtgtggggcatcaagcagctgcaggcccgcgtgctggccgtggagcgctacctgaaggaccagcagctgctgggcatctggggctgcagcggcaagctgatctgcaccaccgccgtgccctggaacgccagctggagcaacaagagcctggaccagatctggaacaacatgacctggatggagtgggagcgcgagatcgacaactacaccagcctgatctacaccctgatcgaggagagccagaaccagcaggagaagaacgagcaggagctgctggagctggacaagtgggccagcctgtggaactggttcgacatcaccaactggctgtggtacatcaagatcttcatcatgatcgtgggcggcctggtgggcctgcgcatcgtgttcgccgtgctgagcatcgtgaaccgcgtgcgccagggctacagccccctgagcttccagacccgcctgcccgccccccgcggccccgaccgccccgagggcatcgaggaggagggcggcgagcgcgaccgcgaccgcagcggccgcctggtggacggcttcctggccctgatctgggtggacctgcgcagcctgtgcctgttcagctaccaccgcctgcgcgacctgctgctgatcgtgacccgcatcgtggagctgctgggccgccgcggctgggaggccctgaagtactggtggaacctgctgcagtactggagccaggagctgaagaacagcgccgtgagcctgctgaacgccaccgccatcgccgtggccgagggcaccgaccgcgtgatcgaggtggtgcagcgggcctgccgcgccatcctgcacatcccccgccgcatccgccagggcctggagcgggccctgctgtaatag

SEQ ID NO: 31 - protein, 842 aa, artificial
  artificial consensus sequence
MRVKGIRKNYQHLWRWGTMLLGMLMICSAAEQLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNATNTTSSSWETMEKGEIKNCSFNITTSIRDKVQKEYALFYNLDVVPIDNASYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSINIGPGRALYTTGEIIGDIRQAHCNISRAKWNNTLKQIVIKLREQFGNKTIVFNQSSGGDPEIVMHSFNCGGEFFYCNSTQLFTWNDTRKLNNTGRNITLPCRIKQIINMWQEVGKAMYAPPIRGQIRCSSNITGLLLTRDGGKDTNGTEIFRPGGGDMRDNWRSELYKYKVVKIEPLGVAPTKAKRRVVQREKRAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTAVPWNASWSNKSLDQIWNNMTWMEWEREIDNYTSLIYTLIEESQNQQEKNEQELLELDKWASLWNWFDITNWLWYIKIFIMIVGGLVGLRIVFAVLSIVNRVRQGYSPLSFQTRLPAPRGPDRPEGIEEEGGERDRDRSGRLVDGFLALIWVDLRSLCLFSYHRLRDLLLIVTRIVELLGRRGWEALKYWWNLLQYWSQELKNSAVSLLNATAIAVAEGTDRVIEVVQRACRAILHIPRRIRQGLERALL

SEQ ID NO: 32 - DNA, 621 bp, artificial
  artificial consensus sequence
atgaagtggagcaagagcagcgtggtgggctggcccaccgtgcgcgagcgcatgcgccgcgccgaggagcccgccgccgacggcgtgggcgccgtgagccgcgacctggagaagcacggcgccatcaccagcagcaacaccgccgccaacaacgccgactgcgcctggctggaggcccaggaggaggaggaagtgggcttccccgtgcgcccccaggtgcccctgcgccccatgacctacaaggccgccgtggacctgagccacttcctgaaggagaagggcggcctggagggcctgatctacagccagaagcgccaggacatcctggacctgtgggtgtaccacacccagggctacttccccgactggcagaactacacccccggccccggcatccgctaccccctgaccttcggctggtgcttcaagctggtgcccgtggagcccgagaaggtggaggaggccaacgagggcgagaacaacagcctgctgcaccccatgagcctgcacggcatggacgaccccgagaaggaggtgctggtgtggaagttcgacagccgcctggccttccaccacatggcccgcgagctgcaccccgagtactacaaggactgctaatag

SEQ ID NO: 33 - protein, 205 aa, artificial
  artificial consensus sequence
MKWSKSSVVGWPTVRERMRRAEEPAADGVGAVSRDLEKHGAITSSNTAANNADCAWLEAQEEEEVGFPVRPQVPLRPMTYKAAVDLSHFLKEKGGLEGLIYSQKRQDILDLWVYHTQGYFPDWQNYTPGPGIRYPLTFGWCFKLVPVEPEKVEEANEGENNSLLHPMSLHGMDDPEKEVLVWKFDSRLAFHHMARELHPEYYKDC

SEQ ID NO: 34 - DNA, 1482 bp, artificial
  artificial consensus sequence
atgggcgcccgcgccagcatcctgcgcggcggcaagctggacacctgggagaagatccgcctgcgccccggcggcaagaagcgctacatgctgaagcacctggtgtgggccagccgcgagctggagcgcttcgccctgaaccccggcctgctggagaccagcgagggctgcaagcagatcatgaagcagctgcagcccgccctgcagaccggcaccgaggagctgaagagcctgtacaacaccgtggccaccctgtactgcgtgcacgagggcatcgaggtgcgggacaccaaggaggccctggacaagatcgaggaggagcagaacaagagccagcagaaaacccagcaggccgaggccgccgacggcaaggtgtcccagaactaccccatcgtgcagaacctgcagggccagatggtgcaccaggccatcagcccccgcaccctgaacgcctgggtgaaggtgatcgaggagaaggccttcagccccgaggtgatccccatgttcaccgccctgagcgagggcgccaccccccaggacctgaacaccatgctgaacaccgtgggcggccaccaggccgccatgcagatgctgaaggacaccatcaacgaggaggccgccgagtgggaccgcctgcaccccgtgcacgccggccccgtggcccccggccagatgcgcgagccccgcggcagcgacatcgccggcaccacctccaccctgcaggagcagatcgcctggatgaccagcaacccccctatccccgtgggcgacatctacaagcgctggatcatcctgggcctgaacaagatcgtgcgcatgtacagccccgtgagcatcctggacatcaagcagggccccaaggagcccttccgcgactacgtggaccgcttcttcaagaccctgcgggccgagcaggccacccaggacgtgaagaactggatgaccgacaccctgctggtgcagaacgccaaccccgactgcaagaccatcctgcgggccctgggccccggcgccagcctggaggagatgatgaccgcctgccagggcgtgggcggccccagccacaaggcccgcgtgctggccgaggccatgagccaggccaacaacaccaacatcatgatgcagcgcagcaacttcaagggcccccgccgcatcgtgaagtgcttcaactgcggcaaggagggccacatcgcccgcaactgccgcgccccccgcaagaagggctgctggaagtgcgggaaggaggggcaccagatgaaggactgcaccgagcgccaggccaacttcctgggcaagatctggccctcccacaagggccgccccggcaacttcctgcagagccgccccgagcccaccgcccctcccgccgagagcttccgcttcgaggagaccacccccgcccccaagcaggagcccaaggaccgcgagcccctgaccagcctgaagagcctgttcggcagcgaccccctgagccagtaatag

SEQ ID NO: 35 - protein, 492 aa, artificial
  artificial sequence
MGARASILRGGKLDTWEKIRLRPGGKKRYMLKHLVWASRELERFALNPGLLETSEGCKQIMKQLQPALQTGTEELKSLYNTVATLYCVHEGIEVRDTKEALDKIEEEQNKSQQKTQQAEAADGKVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGASLEEMMTACQGVGGPSHKARVLAEAMSQANNTNIMMQRSNFKGPRRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLKSLFGSDPLSQ

SEQ ID NO: 36 - DNA, 3000 bp, artificial
  artificial consensus sequence
atgttcttccgcgagaacctggccttcccgcagggcgaggcccgcgagttccccagcgagcagacccgcgccaacagccccacctcccgcgagctgcaggtgcggggcgacaacccccgcagcgaggccggcgccgagcgccagggcaccctgaacttcccgcagatcaccctgtggcagcgccccctggtgagcatcaaggtggggggccagatcaaggaggccctgctggacaccggcgccgacgacaccgtgctggaggagatcaacctgcccggcaagtggaagcccaagatgatcggcggcatcggcggcttcatcaaggtgcggcagtacgaccagatccccatcgagatctgcggcaagaaggccatcggcaccgtgctcgtgggccccacccccgtgaacatcatcggccgcaacatgctgacccagctgggctgcaccctcaacttccccatcagccccatcgagaccgtgcccgtgaagctgaagcccggcatggacggccccaaggtgaagcagtggcccctgaccgaggagaagatcaaggccctgaccgccatctgcgaggagatggagaaggagggcaagatcaccaagatcggccccgagaacccctacaacacccccgtgttcgccatcaagaagaaggacagcaccaagtggcgcaagctcgtggacttccgcgagctgaacaagcgcacccaggacttctgggaggtgcagctgggcatcccccaccccgccggcctgaagaagaagaagagcgtgaccgtgctggacgtgggcgacgcctacttcagcgtgcccctggacgaggacttccgcaagtacaccgccttcaccatccccagcatcaacaacgagacccccggcatccgctaccagtacaacgtgctgccccagggctggaagggcagccccgccatcttccagagcagcatgaccaagatcctggagcccttccgcgcccagaaccccgagatcgtgatctaccagtacatgaacgacctgtacgtgggcagcgacctggagatcggccagcaccgcgccaagatcgaggagctgcgcgagcacctgctgaagtggggcttcaccacccccgacaagaagcaccagaaggagccccccttcctgtggatgggctacgagctgcaccccgacaagtggaccgtgcagcccatccagctgcccgagaaggacagctggaccgtgaacgacatccagaagctcgtgggcaagctgaactgggccagccagatctaccccggcatcaaggtgaggcagctgtgcaagctgctgcgcggcgccaaggccctcaccgacatcgtgcccctcaccgaggaggccgagctggagctggccgagaaccgcgagatcctgaaggagcccgtgcacggcgtgtactacgaccccagcaaggacctgatcgccgagatccagaagcagggcgaccagtggacctaccagatctaccaggagcccttcaagaacctcaagaccggcaagtacgccaagatgcgcaccgcccacaccaacgacgtgaagcagctgaccgaggccgtgcagaagatcgcgatggagagcatcgtgatctggggcaagacccccaagttccgcctgcccatccagaaggagacctgggagacctggtggaccgactactggcaggccacctggatccccgagtgggagttcgtgaacacccctcccctggtgaagctgtggtatcagctggagaaggagcccatcgccggcgccgagaccttctacgtggacggcgccgccaaccgcgagaccaagatcggcaaggccggctacgtgaccgaccgcggccgccagaagatcgtgagcctgaccgagaccaccaaccagaaaaccgagctgcaggccatccagctggcgctgcaggacagcggcagcgaggtgaacatcgtgaccgacagccagtacgccctgggcatcatccaggcccagcccgacaagagcgagagcgagctggtgaaccagatcatcgagcagctgatcaagaaggagcgcgtgtacctgagctgggtgcccgcccacaagggcatcggcggcaacgagcaggtggacaagctggtgagcagcggcatccgcaaggtgctgttcctggacggcatcgacaaggcccaggaggagcacgagaagtaccacagcaactggcgggcgatggccagcgagttcaacctgccccccatcgtggccaaggagatcgtggccagctgcgacaagtgccagctgaagggcgaggccatgcacggccaggtggactgcagccccggcatctggcagctggactgcacccacctggagggcaagatcatcctggtggccgtgcacgtggccagcggctacatcgaggccgaggtgatccccgccgagaccggccaggagaccgcctacttcatcctgaagctggccggccgctggcccgtgaaggtgatccacaccgacaacggcagcaacttcaccagcgccgccgtgaaggccgcctgttggtgggccggcatccagcaggagttcggcatcccctacaacccccagagccagggcgtggtggagagcatgaacaaggagctgaagaagatcatcggccaggtgcgggaccaggccgagcacctcaagaccgccgtgcagatggccgtgttcatccacaacttcaagcgcaagggcggcatcggcgggtacagcgccggcgagcgcatcatcgacatcatcgccaccgacatccagaccaaggagctgcagaagcagatcatcaagatccagaacttccgcgtgtactaccgcgacagccgcgaccccatctggaagggccccgccaagctgctgtggaagggcgagggcgccgtggtgatccaggacaacagcgacatcaaggtggtgccccgccgcaaggccaagatcatcaaggactacggcaagcagatggccggcgccgactgcgtggccggccgccaggacgaggactaa

SEQ ID NO: 37 - protein, 999 aa, artificial
  artificial consensus sequence
MFFRENLAFPQGEAREFPSEQTRANSPTSRELQVRGDNPRSEAGAERQGTLNFPQITLWQRPLVSIKVGGQIKEALLDTGADDTVLEEINLPGKWKPKMIGGIGGFIKVRQYDQIPIEICGKKAIGTVLVGPTPVNIIGRNMLTQLGCTLNFPISPIETVPVKLKPGMDGPKVKQWPLTEEKIKALTAICEEMEKEGKITKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRAQNPEIVIYQYMNDLYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKEPIAGAETFYVDGAANRETKIGKAGYVTDRGRQKIVSLTETTNQKTELQAIQLALQDSGSEVNIVTDSQYALGIIQAQPDKSESELVNQIIEQLIKKERVYLSWVPAHKGIGGNEQVDKLVSSGIRKVLFLDGIDKAQEEHEKYHSNWRAMASEFNLPPIVAKEIVASCDKCQLKGEAMHGQVDCSPGIWQLDCTHLEGKIILVAVHVASGYIEAEVIPAETGQETAYFILKLAGRWPVKVIHTDNGSNFTSAAVKAACWWAGIQQEFGIPYNPQSQGVVESMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKAKIIKDYGKQMAGADCVAGRQDED

SEQ ID NO: 38 - DNA, 2403 bp, artificial
  artificial consensus sequence
atgcgcgtgatgggcatccagcgcaactgccagcagtggtggatctggggcatcctgggcttctggatgctgatgatctgcaacgtgatgggcaacctgtgggtgaccgtgtactacggcgtgcccgtgtggaaggaggccaagaccaccctgttctgcgccagcgacgccaaggcctacgagaccgaggtgcacaacgtgtgggccacccacgcctgcgtgcccaccgaccccaacccccaggagatcgtgctggagaacgtgaccgagaacttcaacatgtggaagaacgacatggtggaccagatgcacgaggacatcatcagcctgtgggaccagagcctgaagccctgcgtgaagctgacccccctgtgcgtgaccctgaactgcaccaacgcggccgcgaactgcaacaccagcgccatcacccaggcctgccccaaggtgtccttcgaccccatccccatccactactgcgcccccgccggctacgccatcctgaagtgcaacaacaagaccttcaacggcaccggcccctgcaacaacgtgagcaccgtgcagtgcacccacggcatcaagcccgtggtgagcacccagctgctgctgaacggcagcctggccgaggaggagatcatcatccgcagcgagaacctgaccaacaacgccaagaccatcatcgtgcacctgaacgagagcgtggagatcgtgtgcacccgccccaacaacaacacccgcaagagcatccgcatcggccccggccagaccttctacgccaccggcgacatcatcggcgacatccgccaggcccactgcaacatcagcggcaccaagtggaacaagaccctgcagcgcgtgagcgagaagctggccgagcacttccccaacaagaccatcaagttcgcccccagcagcggcggcgacctggagatcaccacccacagcttcaactgccgcggcgagttcttctactgcaacaccagcaagctgttcaacagcacctacaacagcaacagcaccgacaacgccaacagcaccgacaactccaccatcaccctgccctgccgcatcaagcagatcatcaacatgtggcagggcgtgggccaggccatctacgcccctcccatccgcggcaacatcacctgcaagtccaacatcaccggcatcctgctgacccgcgacggcggcagcgacgccaacgagaccgagaccttccgccccggcggcggcgacatgcgcgacaactggcgcagcgagctgtacaagtacaaggtggtggagatcaagcccctgggcatcgcccccaccaaggccaagcgccgcgtggtggagcgcgagaagcgggccgtgggcatcggcgccgtgttcctgggcttcctgggcgccgccggcagcacgatgggcgccgccagcatcaccctgaccgtgcaggcccgccagctgctgagcggcatcgtgcagcagcagagcaacctgctgcgggccatcgaagcccagcagcacatgctgcagctgaccgtgtggggcatcaagcagctgcagacccgcgtgctggccatcgagcgctacctgaaggaccagcagctgctgggcatctggggctgcagcggcaagctgatctgcaccaccgccgtgccctggaacagcagctggagcaacaagagccaggccgacatctgggacagcatgacctggatgcagtgggacaaggagatcagcaactacaccggcaccatctaccgcctgctggaggagagccagaaccagcaggagaagaacgagaaggacctgctggccctggacagctggcagaacctgtggaactggttcagcatcaccaactggctgtggtacatcaagatcttcatcatgatcgtgggcggcctgatcggcctgcgcatcatcttcgccgtgctgagcatcgtgaaccgcgtgcgccagggctacagccccctgagcttccagaccctgacccccaacccccgcggccccgaccgcctgggccgcatcgaggaggagggcggcgagcaggacaaggaccgcagcatccgcctggtgagcggcttcctggccctggcctgggacgacctgcgcagcctgtgcctgttcagctaccaccgcctgcgcgacctgatcctgatcgccgcccgcgccgtggagctgctgggccgcagcagcctgcggggcctgcagcgcggctgggagaccctgaagtacctgggcagcctggtgcagtactggggcctggagctgaagaagagcgccatcagcctgctggacaccaccgccatcgccgtggccgagggcaccgaccgcatcctggagctgatccagcgcatctgccgcgccatccgcaacatcccccgccgcatccgccagggcttcgaggccgccctgcagtaatag

SEQ ID NO: 39 - protein, 799 aa, artificial
  artificial consensus sequence
MRVMGIQRNCQQWWIWGILGFWMLMICNVMGNLWVTVYYGVPVWKEAKTTLFCASDAKAYETEVHNVWATHACVPTDPNPQEIVLENVTENFNMWKNDMVDQMHEDIISLWDQSLKPCVKLTPLCVTLNCTNAAANCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISGTKWNKTLQRVSEKLAEHFPNKTIKFAPSSGGDLEITTHSFNCRGEFFYCNTSKLFNSTYNSNSTDNANSTDNSTITLPCRIKQIINMWQGVGQAIYAPPIRGNITCKSNITGILLTRDGGSDANETETFRPGGGDMRDNWRSELYKYKVVEIKPLGIAPTKAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQADIWDSMTWMQWDKEISNYTGTIYRLLEESQNQQEKNEKDLLALDSWQNLWNWFSITNWLWYIKIFIMIVGGLIGLRIIFAVLSIVNRVRQGYSPLSFQTLTPNPRGPDRLGRIEEEGGEQDKDRSIRLVSGFLALAWDDLRSLCLFSYHRLRDLILIAARAVELLGRSSLRGLQRGWETLKYLGSLVQYWGLELKKSAISLLDTTAIAVAEGTDRILELIQRICRAIRNIPRRIRQGFEAALQ

SEQ ID NO: 40 - DNA, 636 bp, artificial
  artificial consensus sequence
atggccgccaagtggtcaaaatgtagtgtgggatggcctgctgtaagagaaagaatgcgccgcactgagccagcagcagaggaggcagcagagggagtaggagcagcatctcaagacttagataaacacggggcacttacaagcagcaacacagccgccaataatgctgattgtgcctggctggaagcgcaagaggaggaagaagaggtaggctttccagtcagacctcaggttcctttaagaccaatgacttataagggagcattcgatctcagcttctttttaaaagaaaaggggggactggaagggttaatttacagcaagaagcgccaggagatcctggacctgtgggtgtaccacacccagggcttcttccccgactggcagaactacacccccggccccggcgtgcgctaccccctgaccttcggctggtgcttcaagctggtgcccgtggaccccggcgaggtggaggaggccaacgagggcgagaacaactgcctgctgcaccccatgagccagcacggcatggaggacgaggaccgcgaggtgctgaagtggaagttcgacagccacctggcccgccgccacatggcccgcgagctgcaccccgagtactacaaggactgctaatag

SEQ ID NO: 41 - protein, 210 aa, artificial
  artificial consensus sequence
MAAKWSKCSVGWPAVRERMRRTEPAAEEAAEGVGAASQDLDKHGALTSSNTAANNADCAWLEAQEEEEEVGFPVRPQVPLRPMTYKGAFDLSFFLKEKGGLEGLIYSKKRQEILDLWVYHTQGFFPDWQNYTPGPGVRYPLTFGWCFKLVPVDPGEVEEANEGENNCLLHPMSQHGMEDEDREVLKWKFDSHLARRHMARELHPEYYKDC

SEQ ID NO: 42 - DNA, 4527 bp, artificial
  artificial fusion gene
atgggcgccagagccagcgtgctgagcggcggcaagctggacgcctgggagaagatcagactgaggcctggcggcaagaagaagtaccggctgaagcacctggtgtgggccagcagagagctggagagattcgccctgaaccctagcctgctggagaccgccgagggctgccagcagatcatggagcagctgcagcctgccctgaaaaccggcaccgaggagctgagaagcctgtacaacaccgtggccaccctgtactgcgtgcaccagcggatcgacgtgaaggataccaaggaggccctggacaagatcgaggagatccagaacaagagcaagcagaaaacccagcaggccgctgccgacaccggcaatagcagcaaagtgagccagaactaccccatcgtgcagaacgcccagggccagatggtgcaccagagcctgagccccagaaccctgaatgcctgggtgaaagtgattgaggagaaggccttcagccccgaagtgatccctatgttcagcgccctgagcgagggcgccaccccccaggatctgaacatgatgctgaacatcgtgggcggccaccaggccgccatgcagatgctgaaggacaccatcaatgaggaggccgccgagtgggacagactgcaccccgtgcacgccggacccatcccccctggccagatgagagagcccagaggcagcgacatcgccggcaccacaagcacccctcaggagcagatcggctggatgaccagcaacccccccatccccgtgggcgacatctacaagcggtggatcatcctgggcctgaacaagatcgtgcggatgtacagccctgtgagcatcctggacatcaagcagggccccaaggagcccttcagagactacgtggaccggttcttcaagaccctgagagccgagcaggccacccaggaagtgaagaactggatgaccgagaccctgctggtgcagaatgccaaccccgactgcaagagcatcctgagagccctgggccctggcgccaccctggaggagatgatgaccgcctgccagggcgtgggcggacctggccacaaggccagagtgctggccgaggccatgagccaagtgcagcacaccaacatcatgatgcagcggggcaacttcagaggccagaagcggatcaagtgcttcaactgcggcaaggagggccacctggccagaaactgcagagcccccaggaagaagggctgctggaagtgtggaaaggaaggccaccagatgaaggactgcaccgagaggcaggccaatttcctgggcaagatctggcctagcagcaagggcagacccggcaatttcccccagagcagacccgagcccaccgcccctcccgccgagatcttcggcatgggcgaggagatcaccagccctcctaagcaggagcagaaggacagagagcagaaccctcctagcgtgagcctgaagagcctgttcggcaacgatcccctgagccagaagtctagaaacgccaccatgttcttcagggagaacctggccttccagcagggcgaggccagaaagttcagcagcgagcagaccagagccaatagccccacctccagagatctgtgggacggcggcagagacagcctgcccagcgaggccggagccgagagacagggcaccggccccaccttcagcttccctcagatcaccctgtggcagagacccctggtgaccgtgaagatcggcggccagctgaaggaggctctgctggatacaggcgccgatgataccgtgctggaggacatcaacctgcccggcaagtggaagcctaagatgatcggcggcatcgggggcttcatcaaagtgaagcagtacgaccagatcctgatcgagatctgcggcaagaaggccatcggcaccgtgctggtcggccccacccctgtgaatatcatcggccggaacatgctgacccagatcggctgcaccctgaacttccccatcagccccatcgagaccgtgcctgtgaagctgaagcctggcatggacggccccaaagtgaaacagtggcccctgaccgaggagaagatcaaggccctgacagagatctgcaccgagatggagaaggagggcaagatcagcaagatcggccccgagaacccctacaacacccccatcttcgccatcaagaagaaggacagcaccaagtggcggaaactggtggacttccgggagctgaacaagaggacccaggacttctgggaagtgcagctgggcatcccccaccctgccggcctgaagaagaagaagagcgtgacagtgctggacgtgggcgatgcctacttcagcgtgcccctggacgagagcttcaggaagtacaccgccttcaccatccccagcaccaacaacgagacccccggcatcagataccagtacaacgtgctgcctcagggctggaagggcagccccgccatcttccagagcagcatgaccaagatcctggagcccttcaggagcaagaaccccgagatcatcatctaccagtacatgaacgacctgtacgtgggcagcgacctggagatcggccagcacagagccaagatcgaggagctgagagcccacctgctgagctggggcttcaccacccccgataagaagcaccagaaggagccccctttcctgtggatgggctacgagctgcaccccgataagtggaccgtgcagcccatcaagctgcctgagaaggagagctggaccgtgaacgacatccagaaactggtgggcaagctgaattgggccagccagatctacgccgggatcaaagtgaaacagctgtgcaagctgctgaggggcgccaaagccctgaccgatatcgtgaccctgaccgaagaggccgagctggagctggccgagaacagggagatcctgaaggatcctgtgcacggcgtgtactacgaccccagcaaggatctgatcgccgagatccagaagcagggccaggatcagtggacctaccagatctaccaggagcctttcaagaacctgaaaaccggcaagtacgccaggaagagaagcgcccacaccaacgacgtgaagcagctggccgaagtggtgcagaaagtggtgatggagagcatcgtgatctggggaaagacccccaagttcaagctgcccatccagaaggagacatgggagacctggtggatggattactggcaggccacctggatccccgagtgggagttcgtgaacacccccccactggtgaagctgtggtatcagctggagaaggaccccatcgctggcgccgagaccttctacgtggacggagccgccaatagagagaccaagctgggcaaggccggctacgtgaccgacagaggcagacagaaagtggtgtccctgaccgagaccaccaaccagaaaaccgagctgcacgccatccatctggccctgcaggacagcggcagcgaagtgaacatcgtgaccgactcccagtacgccctgggcatcatccaggcccagcccgacagaagcgagagcgagctggtgaaccagatcatcgagaagctgatcgagaaggacaaagtgtacctgagctgggtgcccgcccacaagggcatcggcggcaacgagcaagtggacaagctggtgagcagcggcatccggaaagtgctgttcctggacggcatcgataaggcccaggaggagcacgagagataccactccaactggagggccatggccagcgacttcaacctgcctcccatcgtggccaaggagatcgtggccagctgcgataagtgtcagctgaagggggaggccatgcacggccaagtggactgcagccctggcatctggcagctggattgcacccacctggagggcaaagtgatcctggtggccgtgcacgtggccagcggctacatcgaggccgaagtgatccccgccgagaccggccaggagaccgcctacttcctgctgaagctggccggcagatggcccgtgaaagtggtgcacaccgacaacggcagcaatttcaccagcgccgctgtgaaggccgcctgttggtgggccaacgtgcagcaggagttcggcatcccctacaaccctcagagccagggcgtggtggagagcatgaacaaggagctgaagaagatcatcggccaagtgagagagcaggccgagcacctgaaaacagccgtgcagatggctgtgttcatccacaacttcaagcggaagggcggcattggcggctacagcgccggagagcggatcatcgacatcatcgccaccgatatccagaccaaggaactgcagaagcagatcacaaagatccagaacttcagagtgtactaccgggacagcagggaccccatctggaagggccctgccaagctgctgtggaagggcgagggcgccgtggtgatccaggacaacagcgacatcaaagtggtgccccggaggaaggccaagatcatccgggactacggcaagcagatggccggcgacgactgcgtggccggcaggcaggatgaggattga

SEQ ID NO: 43 - protein, 1508 aa, artificial
  artificial fusion protien
MGARASVLSGGKLDAWEKIRLRPGGKKKYRLKHLVWASRELERFALNPSLLETAEGCQQIMEQLQPALKTGTEELRSLYNTVATLYCVHQRIDVKDTKEALDKIEEIQNKSKQKTQQAAADTGNSSKVSQNYPIVQNAQGQMVHQSLSPRTLNAWVKVIEEKAFSPEVIPMFSALSEGATPQDLNMMLNIVGGHQAAMQMLKDTINEEAAEWDRLHPVHAGPIPPGQMREPRGSDIAGTTSTPQEQIGWMTSNPPIPVGDIYKRWIILGLNKIVRMYSPVSILDIKQGPKEPFRDYVDRFFKTLRAEQATQEVKNWMTETLLVQNANPDCKSILRALGPGATLEEMMTACQGVGGPGHKARVLAEAMSQVQHTNIMMQRGNFRGQKRIKCFNCGKEGHLARNCRAPRKKGCWKCGKEGHQMKDCTERQANFLGKIWPSSKGRPGNFPQSRPEPTAPPAEIFGMGEEITSPPKQEQKDREQNPPSVSLKSLFGNDPLSQKSRNATMFFRENLAFQQGEARKFSSEQTRANSPTSRDLWDGGRDSLPSEAGAERQGTGPTFSFPQITLWQRPLVTVKIGGQLKEALLDTGADDTVLEDINLPGKWKPKMIGGIGGFIKVKQYDQILIEICGKKAIGTVLVGPTPVNIIGRNMLTQIGCTLNFPISPIETVPVKLKPGMDGPKVKQWPLTEEKIKALTEICTEMEKEGKISKIGPENPYNTPIFAIKKKDSTKWRKLVDFRELNKRTQDFWEVQLGIPHPAGLKKKKSVTVLDVGDAYFSVPLDESFRKYTAFTIPSTNNETPGIRYQYNVLPQGWKGSPAIFQSSMTKILEPFRSKNPEIIIYQYMNDLYVGSDLEIGQHRAKIEELRAHLLSWGFTTPDKKHQKEPPFLWMGYELHPDKWTVQPIKLPEKESWTVNDIQKLVGKLNWASQIYAGIKVKQLCKLLRGAKALTDIVTLTEEAELELAENREILKDPVHGVYYDPSKDLIAEIQKQGQDQWTYQIYQEPFKNLKTGKYARKRSAHTNDVKQLAEVVQKVVMESIVIWGKTPKFKLPIQKETWETWWMDYWQATWIPEWEFVNTPPLVKLWYQLEKDPIAGAETFYVDGAANRETKLGKAGYVTDRGRQKVVSLTETTNQKTELHAIHLALQDSGSEVNIVTDSQYALGIIQAQPDRSESELVNQIIEKLIEKDKVYLSWVPAHKGIGGNEQVDKLVSSGIRKVLFLDGIDKAQEEHERYHSNWRAMASDFNLPPIVAKEIVASCDKCQLKGEAMHGQVDCSPGIWQLDCTHLEGKVILVAVHVASGYIEAEVIPAETGQETAYFLLKLAGRWPVKVVHTDNGSNFTSAAVKAACWWANVQQEFGIPYNPQSQGVVESMNKELKKIIGQVREQAEHLKTAVQMAVFIHNFKRKGGIGGYSAGERIIDIIATDIQTKELQKQITKIQNFRVYYRDSRDPIWKGPAKLLWKGEGAVVIQDNSDIKVVPRRKAKIIRDYGKQMAGDDCVAGRQDED

SEQ ID NO: 44 - DNA, 3078 bp, artificial
  artificial fusion gene
atgcgcgtgatgggcatccagaggaactgccagcacctgtggagatggggcaccatgatcctgggcatgatcatcatctgctctgccgccgagaacctgtgggtgaccgtgtactacggcgtgcccgtgtggaaggacgccgagaccaccctgttctgcgccagcgacgccaaggcctacgataccgaagtgcacaacgtgtgggccacccacgcctgcgtgcctaccgatcccaacccccaggagatcaacctggagaacgtgaccgaggagttcaacatgtggaagaacaacatggtggagcagatgcacaccgacatcatcagcctgtgggaccagagcctgaagccttgcgtgaagctgacccctctgtgcgtgaccctgaactgcagcaacgccgccaactgcaataccagcgccatcacccaggcctgtcccaaagtgagcttcgagcccatccccatccactactgcgcccctgccggcttcgccatcctgaagtgcaaggacaaggagtttaacggcaccggcccctgcaagaacgtgagcaccgtgcagtgcacccacggcatcaagcccgtggtgagcacccagctgctgctgaacggcagcctggccgaggaagaagtgatgatccggagcgagaacatcaccaacaacgccaagaacatcatcgtgcagctgaccaagcccgtgaagatcaactgcacccggcccaacaacaacacccggaagagcatcagaatcggccctggccaggccttctacgccaccggcgacatcatcggcgatatcaggcaggcccactgcaatgtgagccggaccgagtggaacgagaccctgcagaaagtggccaagcagctgcggaagtacttcaacaacaagaccatcatcttcaccaacagcagcggcggagatctggagatcaccacccacagcttcaattgtggcggcgagttcttctactgcaacacctccggcctgttcaacagcacctggaacggcaacggcaccaagaagaagaacagcaccgagagcaacgacaccatcaccctgccctgccggatcaagcagatcatcaatatgtggcagcgcgtgggccaggccatgtacgcccctcccatccagggcgtgatcagatgcgagagcaacatcaccggcctgctgctgaccagagatggcggcgacaacaacagcaagaacgagaccttcagacctggcggcggagacatgagggacaactggcggagcgagctgtacaagtacaaagtggtgaagatcgagcccctgggcgtggcccccaccaaggccaagagaagagtggtggagcgggagaagagagccgtgggcatcggcgccgtgttcctgggcttcctgggagccgccggaagcaccatgggagccgccagcatcaccctgaccgtgcaggccagacagctgctgagcggcattgtgcagcagcagagcaacctgctgagagccatcgaggcccagcagcacctgctgaagctgacagtgtggggcattaagcagctgcaggcccgcgtgctggccgtggagagatacctgaaggaccagcagctgctgggcatctggggctgcagcggcaagctgatctgcaccaccaacgtgccctggaatagcagctggagcaacaagagccagagcgagatctgggacaacatgacctggctgcagtgggacaaggagatcagcaactacaccgatatcatctacaacctgatcgaggagagccagaaccagcaggagaagaacgagcaggatctgctggccctggacaagtgggccaacctgtggaactggttcgacatcagcaactggctgtggtacatcaagatcttcatcatgatcgtgggcggcctgatcggcctgagaatcgtgttcgccgtgctgagcgtgatcaacagagtgcggcagggctacagccccctgagcttccagacccacacccccaaccctggcggcctggacagacccggcagaatcgaggaggagggcggcgagcagggcagagacaggagcatcagactggtgagcggcttcctggccctggcctgggacgacctgagaagcctgtgcctgttcagctaccaccggctgagggacttcatcctgatcgccgccagaaccgtggagctgctgggacacagctccctgaagggcctgagactgggctgggagggcctgaagtacctgtggaatctgctgctgtactggggcagggagctgaagatcagcgccattaacctgctggacaccatcgccatcgccgtggccggctggaccgacagagtgatcgagatcggccagaggatctgcagagccattctgaacatcccccggaggatcagacagggcctggagcgggccctgctgtctagcgctgaacttcgacctgctgaagctggccggcgacgtggagagcaaccccgccccgtttgggccaccatgaagtggagcaagagcagcatcgtgggctggcctgaagtgcgggagcggatcagaagaaccccccctgccgccaagggcgtgggcgccgtgagccaggacctggacaagcacggagccgtgaccagcagcaacatcaaccaccctagctgcgcctggctggaggcccaggaggaggaggaagtgggcttccctgtgagaccccaagtgcccctgagacccatgacctacaagggcgccttcgacctgagccacttcctgaaggagaagggcggcctggacggcctgatctacagcaagaagcggcaggagatcctggatctgtgggtgtaccacacccagggctacttccccgactggcagaattacacccctggccctggcatcagataccctctgaccttcggctggtgcttcaagctggtgcccgtggaccccgacgaagtggaggaggccaccgagggcgagaacaatagcctgctgcaccccatctgccagcacggcatggacgatgaggagcgggaagtgctgatgtggaagttcgacagcaggctggccctgaagcacagagccagagagctgcaccccgagttctacaaggactgctga

SEQ ID NO: 45 - protein, 1025 aa, artificial
  artificial fusion protein
MRVMGIQRNCQHLWRWGTMILGMIIICSAAENLWVTVYYGVPVWKDAETTLFCASDAKAYDTEVHNVWATHACVPTDPNPQEINLENVTEEFNMWKNNMVEQMHTDIISLWDQSLKPCVKLTPLCVTLNCSNAANCNTSAITQACPKVSFEPIPIHYCAPAGFAILKCKDKEFNGTGPCKNVSTVQCTHGIKPVVSTQLLLNGSLAEEEVMIRSENITNNAKNIIVQLTKPVKINCTRPNNNTRKSIRIGPGQAFYATGDIIGDIRQAHCNVSRTEWNETLQKVAKQLRKYFNNKTIIFTNSSGGDLEITTHSFNCGGEFFYCNTSGLFNSTWNGNGTKKKNSTESNDTITLPCRIKQIINMWQRVGQAMYAPPIQGVIRCESNITGLLLTRDGGDNNSKNETFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQSEIWDNMTWLQWDKEISNYTDIIYNLIEESQNQQEKNEQDLLALDKWANLWNWFDISNWLWYIKIFIMIVGGLIGLRIVFAVLSVINRVRQGYSPLSFQTHTPNPGGLDRPGRIEEEGGEQGRDRSIRLVSGFLALAWDDLRSLCLFSYHRLRDFILIAARTVELLGHSSLKGLRLGWEGLKYLWNLLLYWGRELKISAINLLDTIAIAVAGWTDRVIEIGQRICRAILNIPRRIRQGLERALLSSAELRPAEAGRRRGEQPRPVWATMKWSKSSIVGWPEVRERIRRTPPAAKGVGAVSQDLDKHGAVTSSNINHPSCAWLEAQEEEEVGFPVRPQVPLRPMTYKGAFDLSHFLKEKGGLDGLIYSKKRQEILDLWVYHTQGYFPDWQNYTPGPGIRYPLTFGWCFKLVPVDPDEVEEATEGENNSLLHPICQHGMDDEEREVLMWKFDSRLALKHRARELHPEFYKDC

SEQ ID NO: 46 - DNA, 5211 bp, artificial
  artificial fusion gene
atgggcgccagagccagcgtgctgagcggcggcaagctggacgcctgggagaagatcagactgaggcctggcggcaagaagaagtaccggctgaagcacctggtgtgggccagcagagagctggagagattcgccctgaaccctagcctgctggagaccgccgagggctgccagcagatcatggagcagctgcagcctgccctgaaaaccggcaccgaggagctgagaagcctgtacaacaccgtggccaccctgtactgcgtgcaccagcggatcgacgtgaaggataccaaggaggccctggacaagatcgaggagatccagaacaagagcaagcagaaaacccagcaggccgctgccgacaccggcaatagcagcaaagtgagccagaactaccccatcgtgcagaacgcccagggccagatggtgcaccagagcctgagccccagaaccctgaatgcctgggtgaaagtgattgaggagaaggccttcagccccgaagtgatccctatgttcagcgccctgagcgagggcgccaccccccaggatctgaacatgatgctgaacatcgtgggcggccaccaggccgccatgcagatgctgaaggacaccatcaatgaggaggccgccgagtgggacagactgcaccccgtgcacgccggacccatcccccctggccagatgagagagcccagaggcagcgacatcgccggcaccacaagcacccctcaggagcagatcggctggatgaccagcaacccccccatccccgtgggcgacatctacaagcggtggatcatcctgggcctgaacaagatcgtgcggatgtacagccctgtgagcatcctggacatcaagcagggccccaaggagcccttcagagactacgtggaccggttcttcaagaccctgagagccgagcaggccacccaggaagtgaagaactggatgaccgagaccctgctggtgcagaatgccaaccccgactgcaagagcatcctgagagccctgggccctggcgccaccctggaggagatgatgaccgcctgccagggcgtgggcggacctggccacaaggccagagtgctggccgaggccatgagccaagtgcagcacaccaacatcatgatgcagcggggcaacttcagaggccagaagcggatcaagtgcttcaactgcggcaaggagggccacctggccagaaactgcagagcccccaggaagaagggctgctggaagtgtggaaaggaaggccaccagatgaaggactgcaccgagaggcaggccaatttcctgggcaagatctggcctagcagcaagggcagacccggcaatttcccccagagcagacccgagcccaccgcccctcccgccgagatcttcggcatgggcgaggagatcaccagccctcctaagcaggagcagaaggacagagagcagaaccctcctagcgtgagcctgaagagcctgttcggcaacgatcccctgagccagaagtctagaaacgccaccatgttcttcagggagaacctggccttccagcagggcgaggccagaaagttcagcagcgagcagaccagagccaatagccccacctccagagatctgtgggacggcggcagagacagcctgcccagcgaggccggagccgagagacagggcaccggccccaccttcagcttccctcagatcaccctgtggcagagacccctggtgaccgtgaagatcggcggccagctgaaggaggctctgctggatacaggcgccgatgataccgtgctggaggacatcaacctgcccggcaagtggaagcctaagatgatcggcggcatcgggggcttcatcaaagtgaagcagtacgaccagatcctgatcgagatctgcggcaagaaggccatcggcaccgtgctggtcggccccacccctgtgaatatcatcggccggaacatgctgacccagatcggctgcaccctgaacttccccatcagccccatcgagaccgtgcctgtgaagctgaagcctggcatggacggccccaaagtgaaacagtggcccctgaccgaggagaagatcaaggccctgacagagatctgcaccgagatggagaaggagggcaagatcagcaagatcggccccgagaacccctacaacacccccatcttcgccatcaagaagaaggacagcaccaagtggcggaaactggtggacttccgggagctgaacaagaggacccaggacttctgggaagtgcagctgggcatcccccaccctgccggcctgaagaagaagaagagcgtgacagtgctggacgtgggcgatgcctacttcagcgtgcccctggacgagagcttcaggaagtacaccgccttcaccatccccagcaccaacaacgagacccccggcatcagataccagtacaacgtgctgcctcagggctggaagggcagccccgccatcttccagagcagcatgaccaagatcctggagcccttcaggagcaagaaccccgagatcatcatctaccagtacatgaacgacctgtacgtgggcagcgacctggagatcggccagcacagagccaagatcgaggagctgagagcccacctgctgagctggggcttcaccacccccgataagaagcaccagaaggagccccctttcctgtggatgggctacgagctgcaccccgataagtggaccgtgcagcccatcaagctgcctgagaaggagagctggaccgtgaacgacatccagaaactggtgggcaagctgaattgggccagccagatctacgccgggatcaaagtgaaacagctgtgcaagctgctgaggggcgccaaagccctgaccgatatcgtgaccctgaccgaagaggccgagctggagctggccgagaacagggagatcctgaaggatcctgtgcacggcgtgtactacgaccccagcaaggatctgatcgccgagatccagaagcagggccaggatcagtggacctaccagatctaccaggagcctttcaagaacctgaaaaccggcaagtacgccaggaagagaagcgcccacaccaacgacgtgaagcagctggccgaagtggtgcagaaagtggtgatggagagcatcgtgatctggggaaagacccccaagttcaagctgcccatccagaaggagacatgggagacctggtggatggattactggcaggccacctggatccccgagtgggagttcgtgaacacccccccactggtgaagctgtggtatcagctggagaaggaccccatcgctggcgccgagaccttctacgtggacggagccgccaatagagagaccaagctgggcaaggccggctacgtgaccgacagaggcagacagaaagtggtgtccctgaccgagaccaccaaccagaaaaccgagctgcacgccatccatctggccctgcaggacagcggcagcgaagtgaacatcgtgaccgactcccagtacgccctgggcatcatccaggcccagcccgacagaagcgagagcgagctggtgaaccagatcatcgagaagctgatcgagaaggacaaagtgtacctgagctgggtgcccgcccacaagggcatcggcggcaacgagcaagtggacaagctggtgagcagcggcatccggaaagtgctgttcctggacggcatcgataaggcccaggaggagcacgagagataccactccaactggagggccatggccagcgacttcaacctgcctcccatcgtggccaaggagatcgtggccagctgcgataagtgtcagctgaagggggaggccatgcacggccaagtggactgcagccctggcatctggcagctggattgcacccacctggagggcaaagtgatcctggtggccgtgcacgtggccagcggctacatcgaggccgaagtgatccccgccgagaccggccaggagaccgcctacttcctgctgaagctggccggcagatggcccgtgaaagtggtgcacaccgacaacggcagcaatttcaccagcgccgctgtgaaggccgcctgttggtgggccaacgtgcagcaggagttcggcatcccctacaaccctcagagccagggcgtggtggagagcatgaacaaggagctgaagaagatcatcggccaagtgagagagcaggccgagcacctgaaaacagccgtgcagatggctgtgttcatccacaacttcaagcggaagggcggcattggcggctacagcgccggagagcggatcatcgacatcatcgccaccgatatccagaccaaggaactgcagaagcagatcacaaagatccagaacttcagagtgtactaccgggacagcagggaccccatctggaagggccctgccaagctgctgtggaagggcgagggcgccgtggtgatccaggacaacagcgacatcaaagtggtgccccggaggaaggccaagatcatccgggactacggcaagcagatggccggcgacgactgcgtggccggcaggcaggatgaggattctagcgctgaacttcgacctgctgaagctggccggcgacgtggagagcaaccccggccccgtttaacccgggccaccatgaagtggagcaagagcagcatcgtgggctggcctgaagtgcgggagcggatcagaagaaccccccctgccgccaagggcgtgggcgccgtgagccaggacctggacaagcacggagccgtgaccagcagcaacatcaaccaccctagctgcgcctggctggaggcccaggaggaggaggaagtgggcttccctgtgagaccccaagtgcccctgagacccatgacctacaagggcgccttcgacctgagccacttcctgaaggagaagggcggcctggacggcctgatctacagcaagaagcggcaggagatcctggatctgtgggtgtaccacacccagggctacttccccgactggcagaattacacccctggccctggcatcagataccctctgaccttcggctggtgcttcaagctggtgcccgtggaccccgacgaagtggaggaggccaccgagggcgagaacaatagcctgctgcaccccatctgccagcacggcatggacgatgaggagcgggaagtgctgatgtggaagttcgacagcaggctggccctgaagcacagagccagagagctgcaccccgagttctacaaggactgctga

SEQ ID NO: 47 - protein, 1736 aa, artificial
  artificial fusion protein
MGARASVLSGGKLDAWEKIRLRPGGKKKYRLKHLVWASRELERFALNPSLLETAEGCQQIMEQLQPALKTGTEELRSLYNTVATLYCVHQRIDVKDTKEALDKIEEIQNKSKQKTQQAAADTGNSSKVSQNYPIVQNAQGQMVHQSLSPRTLNAWVKVIEEKAFSPEVIPMFSALSEGATPQDLNMMLNIVGGHQAAMQMLKDTINEEAAEWDRLHPVHAGPIPPGQMREPRGSDIAGTTSTPQEQIGWMTSNPPIPVGDIYKRWIILGLNKIVRMYSPVSILDIKQGPKEPFRDYVDRFFKTLRAEQATQEVKNWMTETLLVQNANPDCKSILRALGPGATLEEMMTACQGVGGPGHKARVLAEAMSQVQHTNIMMQRGNFRGQKRIKCFNCGKEGHLARNCRAPRKKGCWKCGKEGHQMKDCTERQANFLGKIWPSSKGRPGNFPQSRPEPTAPPAEIFGMGEEITSPPKQEQKDREQNPPSVSLKSLFGNDPLSQKSRNATMFFRENLAFQQGEARKFSSEQTRANSPTSRDLWDGGRDSLPSEAGAERQGTGPTFSFPQITLWQRPLVTVKIGGQLKEALLDTGADDTVLEDINLPGKWKPKMIGGIGGFIKVKQYDQILIEICGKKAIGTVLVGPTPVNIIGRNMLTQIGCTLNFPISPIETVPVKLKPGMDGPKVKQWPLTEEKIKALTEICTEMEKEGKISKIGPENPYNTPIFAIKKKDSTKWRKLVDFRELNKRTQDFWEVQLGIPHPAGLKKKKSVTVLDVGDAYFSVPLDESFRKYTAFTIPSTNNETPGIRYQYNVLPQGWKGSPAIFQSSMTKILEPFRSKNPEIIIYQYMNDLYVGSDLEIGQHRAKIEELRAHLLSWGFTTPDKKHQKEPPFLWMGYELHPDKWTVQPIKLPEKESWTVNDIQKLVGKLNWASQIYAGIKVKQLCKLLRGAKALTDIVTLTEEAELELAENREILKDPVHGVYYDPSKDLIAEIQKQGQDQWTYQIYQEPFKNLKTGKYARKRSAHTNDVKQLAEVVQKVVMESIVIWGKTPKFKLPIQKETWETWWMDYWQATWIPEWEFVNTPPLVKLWYQLEKDPIAGAETFYVDGAANRETKLGKAGYVTDRGRQKVVSLTETTNQKTELHAIHLALQDSGSEVNIVTDSQYALGIIQAQPDRSESELVNQIIEKLIEKDKVYLSWVPAHKGIGGNEQVDKLVSSGIRKVLFLDGIDKAQEEHERYHSNWRAMASDFNLPPIVAKEIVASCDKCQLKGEAMHGQVDCSPGIWQLDCTHLEGKVILVAVHVASGYIEAEVIPAETGQETAYFLLKLAGRWPVKVVHTDNGSNFTSAAVKAACWWANVQQEFGIPYNPQSQGVVESMNKELKKIIGQVREQAEHLKTAVQMAVFIHNFKRKGGIGGYSAGERIIDIIATDIQTKELQKQITKIQNFRVYYRDSRDPIWKGPAKLLWKGEGAVVIQDNSDIKVVPRRKAKIIRDYGKQMAGDDCVAGRQDEDSSAELRPAEAGRRRGEQPRPRLTRATMKWSKSSIVGWPEVRERIRRTPPAAKGVGAVSQDLDKHGAVTSSNINHPSCAWLEAQEEEEVGFPVRPQVPLRPMTYKGAFDLSHFLKEKGGLDGLIYSKKRQEILDLWVYHTQGYFPDWQNYTPGPGIRYPLTFGWCFKLVPVDPDEVEEATEGENNSLLHPICQHGMDDEEREVLMWKFDSRLALKHRARELHPEFYKDC

SEQ ID NO: 48 - DNA, 7683 bp, artificial
  artificial fusion gene
atgggcgccagagccagcgtgctgagcggcggcaagctggacgcctgggagaagatcagactgaggcctggcggcaagaagaagtaccggctgaagcacctggtgtgggccagcagagagctggagagattcgccctgaaccctagcctgctggagaccgccgagggctgccagcagatcatggagcagctgcagcctgccctgaaaaccggcaccgaggagctgagaagcctgtacaacaccgtggccaccctgtactgcgtgcaccagcggatcgacgtgaaggataccaaggaggccctggacaagatcgaggagatccagaacaagagcaagcagaaaacccagcaggccgctgccgacaccggcaatagcagcaaagtgagccagaactaccccatcgtgcagaacgcccagggccagatggtgcaccagagcctgagccccagaaccctgaatgcctgggtgaaagtgattgaggagaaggccttcagccccgaagtgatccctatgttcagcgccctgagcgagggcgccaccccccaggatctgaacatgatgctgaacatcgtgggcggccaccaggccgccatgcagatgctgaaggacaccatcaatgaggaggccgccgagtgggacagactgcaccccgtgcacgccggacccatcccccctggccagatgagagagcccagaggcagcgacatcgccggcaccacaagcacccctcaggagcagatcggctggatgaccagcaacccccccatccccgtgggcgacatctacaagcggtggatcatcctgggcctgaacaagatcgtgcggatgtacagccctgtgagcatcctggacatcaagcagggccccaaggagcccttcagagactacgtggaccggttcttcaagaccctgagagccgagcaggccacccaggaagtgaagaactggatgaccgagaccctgctggtgcagaatgccaaccccgactgcaagagcatcctgagagccctgggccctggcgccaccctggaggagatgatgaccgcctgccagggcgtgggcggacctggccacaaggccagagtgctggccgaggccatgagccaagtgcagcacaccaacatcatgatgcagcggggcaacttcagaggccagaagcggatcaagtgcttcaactgcggcaaggagggccacctggccagaaactgcagagcccccaggaagaagggctgctggaagtgtggaaaggaaggccaccagatgaaggactgcaccgagaggcaggccaatttcctgggcaagatctggcctagcagcaagggcagacccggcaatttcccccagagcagacccgagcccaccgcccctcccgccgagatcttcggcatgggcgaggagatcaccagccctcctaagcaggagcagaaggacagagagcagaaccctcctagcgtgagcctgaagagcctgttcggcaacgatcccctgagccagaagtctagaaacgccaccatgttcttcagggagaacctggccttccagcagggcgaggccagaaagttcagcagcgagcagaccagagccaatagccccacctccagagatctgtgggacggcggcagagacagcctgcccagcgaggccggagccgagagacagggcaccggccccaccttcagcttccctcagatcaccctgtggcagagacccctggtgaccgtgaagatcggcggccagctgaaggaggctctgctggatacaggcgccgatgataccgtgctggaggacatcaacctgcccggcaagtggaagcctaagatgatcggcggcatcgggggcttcatcaaagtgaagcagtacgaccagatcctgatcgagatctgcggcaagaaggccatcggcaccgtgctggtcggccccacccctgtgaatatcatcggccggaacatgctgacccagatcggctgcaccctgaacttccccatcagccccatcgagaccgtgcctgtgaagctgaagcctggcatggacggccccaaagtgaaacagtggcccctgaccgaggagaagatcaaggccctgacagagatctgcaccgagatggagaaggagggcaagatcagcaagatcggccccgagaacccctacaacacccccatcttcgccatcaagaagaaggacagcaccaagtggcggaaactggtggacttccgggagctgaacaagaggacccaggacttctgggaagtgcagctgggcatcccccaccctgccggcctgaagaagaagaagagcgtgacagtgctggacgtgggcgatgcctacttcagcgtgcccctggacgagagcttcaggaagtacaccgccttcaccatccccagcaccaacaacgagacccccggcatcagataccagtacaacgtgctgcctcagggctggaagggcagccccgccatcttccagagcagcatgaccaagatcctggagcccttcaggagcaagaaccccgagatcatcatctaccagtacatgaacgacctgtacgtgggcagcgacctggagatcggccagcacagagccaagatcgaggagctgagagcccacctgctgagctggggcttcaccacccccgataagaagcaccagaaggagccccctttcctgtggatgggctacgagctgcaccccgataagtggaccgtgcagcccatcaagctgcctgagaaggagagctggaccgtgaacgacatccagaaactggtgggcaagctgaattgggccagccagatctacgccgggatcaaagtgaaacagctgtgcaagctgctgaggggcgccaaagccctgaccgatatcgtgaccctgaccgaagaggccgagctggagctggccgagaacagggagatcctgaaggatcctgtgcacggcgtgtactacgaccccagcaaggatctgatcgccgagatccagaagcagggccaggatcagtggacctaccagatctaccaggagcctttcaagaacctgaaaaccggcaagtacgccaggaagagaagcgcccacaccaacgacgtgaagcagctggccgaagtggtgcagaaagtggtgatggagagcatcgtgatctggggaaagacccccaagttcaagctgcccatccagaaggagacatgggagacctggtggatggattactggcaggccacctggatccccgagtgggagttcgtgaacacccccccactggtgaagctgtggtatcagctggagaaggaccccatcgctggcgccgagaccttctacgtggacggagccgccaatagagagaccaagctgggcaaggccggctacgtgaccgacagaggcagacagaaagtggtgtccctgaccgagaccaccaaccagaaaaccgagctgcacgccatccatctggccctgcaggacagcggcagcgaagtgaacatcgtgaccgactcccagtacgccctgggcatcatccaggcccagcccgacagaagcgagagcgagctggtgaaccagatcatcgagaagctgatcgagaaggacaaagtgtacctgagctgggtgcccgcccacaagggcatcggcggcaacgagcaagtggacaagctggtgagcagcggcatccggaaagtgctgttcctggacggcatcgataaggcccaggaggagcacgagagataccactccaactggagggccatggccagcgacttcaacctgcctcccatcgtggccaaggagatcgtggccagctgcgataagtgtcagctgaagggggaggccatgcacggccaagtggactgcagccctggcatctggcagctggattgcacccacctggagggcaaagtgatcctggtggccgtgcacgtggccagcggctacatcgaggccgaagtgatccccgccgagaccggccaggagaccgcctacttcctgctgaagctggccggcagatggcccgtgaaagtggtgcacaccgacaacggcagcaatttcaccagcgccgctgtgaaggccgcctgttggtgggccaacgtgcagcaggagttcggcatcccctacaaccctcagagccagggcgtggtggagagcatgaacaaggagctgaagaagatcatcggccaagtgagagagcaggccgagcacctgaaaacagccgtgcagatggctgtgttcatccacaacttcaagcggaagggcggcattggcggctacagcgccggagagcggatcatcgacatcatcgccaccgatatccagaccaaggaactgcagaagcagatcacaaagatccagaacttcagagtgtactaccgggacagcagggaccccatctggaagggccctgccaagctgctgtggaagggcgagggcgccgtggtgatccaggacaacagcgacatcaaagtggtgccccggaggaaggccaagatcatccgggactacggcaagcagatggccggcgacgactgcgtggccggcaggcaggatgaggattctagcgctgaacttcgacctgctgaagctggccggcgacgtggagagcaaccccggccccgtttgggtttaaacgccaccatgcgcgtgatgggcatccagaggaactgccagcacctgtggagatggggcaccatgatcctgggcatgatcatcatctgctctgccgccgagaacctgtgggtgaccgtgtactacggcgtgcccgtgtggaaggacgccgagaccaccctgttctgcgccagcgacgccaaggcctacgataccgaagtgcacaacgtgtgggccacccacgcctgcgtgcctaccgatcccaacccccaggagatcaacctggagaacgtgaccgaggagttcaacatgtggaagaacaacatggtggagcagatgcacaccgacatcatcagcctgtgggaccagagcctgaagccttgcgtgaagctgacccctctgtgcgtgaccctgaactgcagcaacgccgccaactgcaataccagcgccatcacccaggcctgtcccaaagtgagcttcgagcccatccccatccactactgcgcccctgccggcttcgccatcctgaagtgcaaggacaaggagtttaacggcaccggcccctgcaagaacgtgagcaccgtgcagtgcacccacggcatcaagcccgtggtgagcacccagctgctgctgaacggcagcctggccgaggaagaagtgatgatccggagcgagaacatcaccaacaacgccaagaacatcatcgtgcagctgaccaagcccgtgaagatcaactgcacccggcccaacaacaacacccggaagagcatcagaatcggccctggccaggccttctacgccaccggcgacatcatcggcgatatcaggcaggcccactgcaatgtgagccggaccgagtggaacgagaccctgcagaaagtggccaagcagctgcggaagtacttcaacaacaagaccatcatcttcaccaacagcagcggcggagatctggagatcaccacccacagcttcaattgtggcggcgagttcttctactgcaacacctccggcctgttcaacagcacctggaacggcaacggcaccaagaagaagaacagcaccgagagcaacgacaccatcaccctgccctgccggatcaagcagatcatcaatatgtggcagcgcgtgggccaggccatgtacgcccctcccatccagggcgtgatcagatgcgagagcaacatcaccggcctgctgctgaccagagatggcggcgacaacaacagcaagaacgagaccttcagacctggcggcggagacatgagggacaactggcggagcgagctgtacaagtacaaagtggtgaagatcgagcccctgggcgtggcccccaccaaggccaagagaagagtggtggagcgggagaagagagccgtgggcatcggcgccgtgttcctgggcttcctgggagccgccggaagcaccatgggagccgccagcatcaccctgaccgtgcaggccagacagctgctgagcggcattgtgcagcagcagagcaacctgctgagagccatcgaggcccagcagcacctgctgaagctgacagtgtggggcattaagcagctgcaggcccgcgtgctggccgtggagagatacctgaaggaccagcagctgctgggcatctggggctgcagcggcaagctgatctgcaccaccaacgtgccctggaatagcagctggagcaacaagagccagagcgagatctgggacaacatgacctggctgcagtgggacaaggagatcagcaactacaccgatatcatctacaacctgatcgaggagagccagaaccagcaggagaagaacgagcaggatctgctggccctggacaagtgggccaacctgtggaactggttcgacatcagcaactggctgtggtacatcaagatcttcatcatgatcgtgggcggcctgatcggcctgagaatcgtgttcgccgtgctgagcgtgatcaacagagtgcggcagggctacagccccctgagcttccagacccacacccccaaccctggcggcctggacagacccggcagaatcgaggaggagggcggcgagcagggcagagacaggagcatcagactggtgagcggcttcctggccctggcctgggacgacctgagaagcctgtgcctgttcagctaccaccggctgagggacttcatcctgatcgccgccagaaccgtggagctgctgggacacagctccctgaagggcctgagactgggctgggagggcctgaagtacctgtggaatctgctgctgtactggggcagggagctgaagatcagcgccattaacctgctggacaccatcgccatcgccgtggccggctggaccgacagagtgatcgagatcggccagaggatctgcagagccattctgaacatcccccggaggatcagacagggcctggagcgggccctgctgtctagcgctgaacttcgacctgctgaagctggccggcgacgtggagagcaaccccgccccgtttgggccaccatgaagtggagcaagagcagcatcgtgggctggcctgaagtgcgggagcggatcagaagaaccccccctgccgccaagggcgtgggcgccgtgagccaggacctggacaagcacggagccgtgaccagcagcaacatcaaccaccctagctgcgcctggctggaggcccaggaggaggaggaagtgggcttccctgtgagaccccaagtgcccctgagacccatgacctacaagggcgccttcgacctgagccacttcctgaaggagaagggcggcctggacggcctgatctacagcaagaagcggcaggagatcctggatctgtgggtgtaccacacccagggctacttccccgactggcagaattacacccctggccctggcatcagataccctctgaccttcggctggtgcttcaagctggtgcccgtggaccccgacgaagtggaggaggccaccgagggcgagaacaatagcctgctgcaccccatctgccagcacggcatggacgatgaggagcgggaagtgctgatgtggaagttcgacagcaggctggccctgaagcacagagccagagagctgcaccccgagttctacaaggactgctga

SEQ ID NO: 49 - protein, 2560 aa, artificial
  artificial fusion protein
MGARASVLSGGKLDAWEKIRLRPGGKKKYRLKHLVWASRELERFALNPSLLETAEGCQQIMEQLQPALKTGTEELRSLYNTVATLYCVHQRIDVKDTKEALDKIEEIQNKSKQKTQQAAADTGNSSKVSQNYPIVQNAQGQMVHQSLSPRTLNAWVKVIEEKAFSPEVIPMFSALSEGATPQDLNMMLNIVGGHQAAMQMLKDTINEEAAEWDRLHPVHAGPIPPGQMREPRGSDIAGTTSTPQEQIGWMTSNPPIPVGDIYKRWIILGLNKIVRMYSPVSILDIKQGPKEPFRDYVDRFFKTLRAEQATQEVKNWMTETLLVQNANPDCKSILRALGPGATLEEMMTACQGVGGPGHKARVLAEAMSQVQHTNIMMQRGNFRGQKRIKCFNCGKEGHLARNCRAPRKKGCWKCGKEGHQMKDCTERQANFLGKIWPSSKGRPGNFPQSRPEPTAPPAEIFGMGEEITSPPKQEQKDREQNPPSVSLKSLFGNDPLSQKSRNATMFFRENLAFQQGEARKFSSEQTRANSPTSRDLWDGGRDSLPSEAGAERQGTGPTFSFPQITLWQRPLVTVKIGGQLKEALLDTGADDTVLEDINLPGKWKPKMIGGIGGFIKVKQYDQILIEICGKKAIGTVLVGPTPVNIIGRNMLTQIGCTLNFPISPIETVPVKLKPGMDGPKVKQWPLTEEKIKALTEICTEMEKEGKISKIGPENPYNTPIFAIKKKDSTKWRKLVDFRELNKRTQDFWEVQLGIPHPAGLKKKKSVTVLDVGDAYFSVPLDESFRKYTAFTIPSTNNETPGIRYQYNVLPQGWKGSPAIFQSSMTKILEPFRSKNPEIIIYQYMNDLYVGSDLEIGQHRAKIEELRAHLLSWGFTTPDKKHQKEPPFLWMGYELHPDKWTVQPIKLPEKESWTVNDIQKLVGKLNWASQIYAGIKVKQLCKLLRGAKALTDIVTLTEEAELELAENREILKDPVHGVYYDPSKDLIAEIQKQGQDQWTYQIYQEPFKNLKTGKYARKRSAHTNDVKQLAEVVQKVVMESIVIWGKTPKFKLPIQKETWETWWMDYWQATWIPEWEFVNTPPLVKLWYQLEKDPIAGAETFYVDGAANRETKLGKAGYVTDRGRQKVVSLTETTNQKTELHAIHLALQDSGSEVNIVTDSQYALGIIQAQPDRSESELVNQIIEKLIEKDKVYLSWVPAHKGIGGNEQVDKLVSSGIRKVLFLDGIDKAQEEHERYHSNWRAMASDFNLPPIVAKEIVASCDKCQLKGEAMHGQVDCSPGIWQLDCTHLEGKVILVAVHVASGYIEAEVIPAETGQETAYFLLKLAGRWPVKVVHTDNGSNFTSAAVKAACWWANVQQEFGIPYNPQSQGVVESMNKELKKIIGQVREQAEHLKTAVQMAVFIHNFKRKGGIGGYSAGERIIDIIATDIQTKELQKQITKIQNFRVYYRDSRDPIWKGPAKLLWKGEGAVVIQDNSDIKVVPRRKAKIIRDYGKQMAGDDCVAGRQDEDSSAELRPAEAGRRRGEQPRPRLGLNATMRVMGIQRNCQHLWRWGTMILGMIIICSAAENLWVTVYYGVPVWKDAETTLFCASDAKAYDTEVHNVWATHACVPTDPNPQEINLENVTEEFNMWKNNMVEQMHTDIISLWDQSLKPCVKLTPLCVTLNCSNAANCNTSAITQACPKVSFEPIPIHYCAPAGFAILKCKDKEFNGTGPCKNVSTVQCTHGIKPVVSTQLLLNGSLAEEEVMIRSENITNNAKNIIVQLTKPVKINCTRPNNNTRKSIRIGPGQAFYATGDIIGDIRQAHCNVSRTEWNETLQKVAKQLRKYFNNKTIIFTNSSGGDLEITTHSFNCGGEFFYCNTSGLFNSTWNGNGTKKKNSTESNDTITLPCRIKQIINMWQRVGQAMYAPPIQGVIRCESNITGLLLTRDGGDNNSKNETFRPGGGDMRDNWRSELYKYKVVKIEPLGVAPTKAKRRVVEREKRAVGIGAVFLGFLGAAGSTMGAASITLTVQARQLLSGIVQQQSNLLRAIEAQQHLLKLTVWGIKQLQARVLAVERYLKDQQLLGIWGCSGKLICTTNVPWNSSWSNKSQSEIWDNMTWLQWDKEISNYTDIIYNLIEESQNQQEKNEQDLLALDKWANLWNWFDISNWLWYIKIFIMIVGGLIGLRIVFAVLSVINRVRQGYSPLSFQTHTPNPGGLDRPGRIEEEGGEQGRDRSIRLVSGFLALAWDDLRSLCLFSYHRLRDFILIAARTVELLGHSSLKGLRLGWEGLKYLWNLLLYWGRELKISAINLLDTIAIAVAGWTDRVIEIGQRICRAILNIPRRIRQGLERALLSSAELRPAEAGRRRGEQPRPVWATMKWSKSSIVGWPEVRERIRRTPPAAKGVGAVSQDLDKHGAVTSSNINHPSCAWLEAQEEEEVGFPVRPQVPLRPMTYKGAFDLSHFLKEKGGLDGLIYSKKRQEILDLWVYHTQGYFPDWQNYTPGPGIRYPLTFGWCFKLVPVDPDEVEEATEGENNSLLHPICQHGMDDEEREVLMWKFDSRLALKHRARELHPEFYKDC

SEQ ID NO: 50 - DNA, 4533 bp, artificial
  artificial fusion gene
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgaggtgaaggacaccaaggaggccctggagaagatcgaggaggagcagaacaagagcaagaagaaggcccagcaggccgccgccgacaccggcaacagcagccaagtgagccagaactaccccatcgtgcagaacctgcagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcctgcaccccgtgcacgccggccccatcgcccccggccagatgcgcgagccccgcggcagcgacatcgccggcaccacgagcaccctgcaggagcagatcggctggatgaccaacaacccccctatccccgtgggcgagatctacaagcgctggatcatcctgggcctgaacaagatcgtgcgcatgtacagccccacgagcatcctggacatccgccagggccccaaggagcccttccgcgactacgtggaccgcttctacaagaccctgcgggccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagaccgtgaagtgcttcaactgcgggaaggagggccacatcgccaagaactgccgcgccccccgcaagaagggctgctggaagtgcggcaaggaggggcaccagatgaaggactgcaccgagcgccaggccaacttcctgggcaagatctggcccagccacaagggccgccccggcaacttcctgcagagccgccccgagcccaccgcccctcccgaggagagcttccgcttcggcgaggagaccaccacccccagccagaagcaggagcccatcgacaaggagctgtaccccctggccagcctgcgcagcctgttcggcaacgaccccagcagccaggccatgggggccaccatggccttcttccgcgaggacctggccttcccccaaggcaaggcccgcgagttcagcagcgagcagacccgcgccaacagccccacccgccgcgagctgcaggtgtggggccgcgacaacaacagcctgagcgaggccggcgccgaccgccagggcaccgtgagcttcagcttcccccaaatcaccctgtggcagcgccccctggtgaccatcaagatcggcggccagctgaaggaggccctgctggacaccggcgccgacgacaccgtgctggaagagatgaacctgcccggccgctggaagcccaagatgatcggcggcatcggcggcttcatcaaagtgcgccagtacgaccagatcctgatcgagatctgcggccacaaggccatcggcaccgtgctcgtgggccccacccccgtgaacatcatcggccgcaacctgctgacccagatcggctgcaccctgaacttccccatcagccccatcgagaccgtgcccgtgaagctgaagcccggcatggacggccccaaggtgaagcagtggcccctgaccgaggagaagatcaaggccctggtggagatctgcaccgagatggagaaggagggcaagatcagcaagatcggccccgagaacccctacaacacccccgtgttcgccatcaagaagaaggacagcaccaagtggcgcaagctcgtggacttccgcgagctgaacaagcgcacccaggacttctgggaggtgcagctgggcatcccccaccccgccggcctgaagaagaagaagagcgtgaccgtgctggacgtgggcgacgcctacttcagcgtgcccctggacaaggacttccgcaagtacaccgccttcaccatccccagcatcaacaacgagacccccggcatccgctaccagtacaacgtgctgccccagggctggaagggcagccccgccatcttccagagcagcatgaccaagatcctggagcccttccgcaagcagaaccccgacatcgtgatctaccagtacatgaacgacctgtacgtgggcagcgacctggagatcggccagcaccgcaccaagatcgaggagctgcgccagcacctgctgcgctggggcttcaccacccccgacaagaagcaccagaaggagccccccttcctgtggatgggctacgagctgcaccccgacaagtggaccgtgcagcccatcgtgctgcccgagaaggacagctggaccgtgaacgacatccagaagctcgtgggcaagctgaactgggccagccagatctacgccggcatcaaggtgaagcagctgtgcaagctgctgcgcggcaccaaggccctgaccgaggtgatccccctgaccgaggaggccgagctggagctggccgagaaccgcgagatcctgaaggagcccgtgcacggcgtgtactacgaccccagcaaggacctgatcgccgagatccagaagcagggccagggccagtggacctaccagatctaccaggagcccttcaagaacctcaagaccggcaagtacgcccgcatgcgcggcgcccacaccaacgacgtgaagcagctgaccgaggccgtgcagaagatcgccaccgagagcatcgtgatctggggcaagacccccaagttcaagctgcccatccagaaggagacctgggagacctggtggaccgagtactggcaggccacctggatccccgagtgggagttcgtgaacacccctcccctggtgaagctgtggtatcagctggagaaggagcccatcgtgggcgccgagaccttctacgtggacggcgccgccaaccgcgagaccaagctgggcaaggccggctacgtgaccgaccgcggccgccagaaggtggtgagcctgaccgacaccaccaaccaaaagaccgagctgcaggccatccacctggccctgcaggacagcggcctggaggtgaacatcgtgaccgacagccagtacgccctgggcatcatccaggcccagcccgacaagagcgagagcgagctggtgagccagatcatcgagcagctgatcaagaaggagaaggtgtacctggcctgggtgcccgcccacaagggcatcggcggcaacgagcaggtggacaagctggtgagcgccggcatccgcaaggtgctgttcctggacggcatcgacaaggcccaggaggagcacgagaagtaccacagcaactggcgggccatggccagcgacttcaacctgccccccgtggtggccaaggagatcgtggccagctgcgacaagtgccagctgaagggcgaggccatgcacggccaggtggactgcagccccggcatctggcagctggactgcacccacctggagggcaagatcatcctggtggccgtgcacgtggccagcggctacatcgaggccgaggtgatccccgccgagaccggccaggagaccgcctacttcctgctgaagctggccggccgctggcccgtcaagaccatccacaccgacaacggcagcaacttcaccagcaccaccgtgaaggccgcctgttggtgggccggcatcaagcaggagttcggcatcccctacaacccccagagccagggcgtggtggagagcatgaacaaggagctgaagaagatcatcggccaagtgcgcgaccaggccgagcacctcaagaccgccgtgcagatggccgtgttcatccacaacttcaagcgcaagggcgggatcggcggctacagcgccggcgagcgcatcgtggacatcatcgccaccgacatccagaccaaggagctgcagaagcagatcaccaagatccagaacttccgcgtgtactaccgcgacagccgcgaccccctgtggaagggccccgccaagctgctgtggaagggcgagggcgccgtggtgatccaggacaacagcgacatcaaggtggtgccccgccgcaaggccaagatcatccgcgactacggcaagcagatggccggcgacgactgcgtggccagccgccaggacgaggactaa

SEQ ID NO: 51 - protein, 1510 aa, artificial
  artificial fusion protein
MGARASVLSGGELDRWEKIRLRPGGKKKYKLKHIVWASRELERFAVNPGLLETSEGCRQILGQLQPSLQTGSEELRSLYNTVATLYCVHQRIEVKDTKEALEKIEEEQNKSKKKAQQAAADTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGNDPSSQAMGATMAFFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTKILEPFRKQNPDIVIYQYMNDLYVGSDLEIGQHRTKIEELRQHLLRWGFTTPDKKHQKEPPFLWMGYELHPDKWTVQPIVLPEKDSWTVNDIQKLVGKLNWASQIYAGIKVKQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWETWWTEYWQATWIPEWEFVNTPPLVKLWYQLEKEPIVGAETFYVDGAANRETKLGKAGYVTDRGRQKVVSLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKIILVAVHVASGYIEAEVIPAETGQETAYFLLKLAGRWPVKTIHTDNGSNFTSTTVKAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRDPLWKGPAKLLWKGEGAVVIQDNSDIKVVPRRKAKIIRDYGKQMAGDDCVASRQDED

SEQ ID NO: 52 - DNA, 3207 bp, artificial
  artificial fusion gene
atgcgcgtgaagggcatccgcaagaactaccagcacctgtggcgctggggcaccatgctgctgggcatgctgatgatctgcagcgccgccgagcagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggaggtggtgctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccgacctgcgcaacgccaccaacaccacctccagcagctgggagaccatggagaagggcgagatcaagaactgcagcttcaacatcaccacctccatccgcgacaaggtgcagaaggagtacgccctgttctacaacctggacgtggtgcccatcgacaacgccagctaccgcctgatcagctgcaacaccagcgtgatcacccaggcctgccccaaagtgagcttcgagcccatccccatccactactgcgcccccgccggcttcgccatcctgaagtgcaacgacaagaagttcaacggcaccggcccctgcaccaacgtgagcaccgtgcagtgcacccacggcatccgccccgtggtgagcacccagctgctgctgaacggcagcctggccgaggaggaggtggtgatccgcagcgagaacttcaccgacaacgccaagaccatcatcgtgcagctgaacgagagcgtggagatcaactgcacccgccccaacaacaacacccgcaagagcatcaacatcggccccggccgcgccctgtacaccaccggcgagatcatcggcgacatccgccaggcccactgcaacatcagccgcgccaagtggaacaacaccctgaagcagatcgtgatcaagctgcgcgagcagttcggcaacaagaccatcgtgttcaaccagagcagcggcggcgaccccgagatcgtgatgcacagcttcaactgcggcggcgagttcttctactgcaacagcacccagctgttcacctggaacgacacccgcaagctgaacaacaccggccgcaacatcaccctgccctgccgcatcaagcagatcatcaacatgtggcaggaagtgggcaaggccatgtacgcccctcccatccgcggccagatccgctgcagcagcaacatcaccggcctgctgctgacccgcgacggcggcaaggacaccaacggcaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgggccgtgggcatcggcgccatgttcctgggcttcctgggcgccgccggcagcaccatgggcgccgccagcatgaccctgaccgtgcaggcccgccagctgctgagcggcatcgtgcagcagcagaacaacctgctgcgggccatcgaggcccagcagcacctgctgcagctgaccgtgtggggcatcaagcagctgcaggcccgcgtgctggccgtggagcgctacctgaaggaccagcagctgctgggcatctggggctgcagcggcaagctgatctgcaccaccgccgtgccctggaacgccagctggagcaacaagagcctggaccagatctggaacaacatgacctggatggagtgggagcgcgagatcgacaactacaccagcctgatctacaccctgatcgaggagagccagaaccagcaggagaagaacgagcaggagctgctggagctggacaagtgggccagcctgtggaactggttcgacatcaccaactggctgtggtacatcaagatcttcatcatgatcgtgggcggcctggtgggcctgcgcatcgtgttcgccgtgctgagcatcgtgaaccgcgtgcgccagggctacagccccctgagcttccagacccgcctgcccgccccccgcggccccgaccgccccgagggcatcgaggaggagggcggcgagcgcgaccgcgaccgcagcggccgcctggtggacggcttcctggccctgatctgggtggacctgcgcagcctgtgcctgttcagctaccaccgcctgcgcgacctgctgctgatcgtgacccgcatcgtggagctgctgggccgccgcggctgggaggccctgaagtactggtggaacctgctgcagtactggagccaggagctgaagaacagcgccgtgagcctgctgaacgccaccgccatcgccgtggccgagggcaccgaccgcgtgatcgaggtggtgcagcgggcctgccgcgccatcctgcacatcccccgccgcatccgccagggcctggagcgggccctgctgaacctcgacctgctgaagctggccggcgacgtggagagcaaccccggccccgtttgggccaccatgaagtggagcaagagcagcgtggtgggctggcccaccgtgcgcgagcgcatgcgccgcgccgaggagcccgccgccgacggcgtgggcgccgtgagccgcgacctggagaagcacggcgccatcaccagcagcaacaccgccgccaacaacgccgactgcgcctggctggaggcccaggaggaggaggaagtgggcttccccgtgcgcccccaggtgcccctgcgccccatgacctacaaggccgccgtggacctgagccacttcctgaaggagaagggcggcctggagggcctgatctacagccagaagcgccaggacatcctggacctgtgggtgtaccacacccagggctacttccccgactggcagaactacacccccggccccggcatccgctaccccctgaccttcggctggtgcttcaagctggtgcccgtggagcccgagaaggtggaggaggccaacgagggcgagaacaacagcctgctgcaccccatgagcctgcacggcatggacgaccccgagaaggaggtgctggtgtggaagttcgacagccgcctggccttccaccacatggcccgcgagctgcaccccgagtactacaaggactgctaa

SEQ ID NO: 53 - protein, 1068 aa, artificial
  artificial fusion protein
MRVKGIRKNYQHLWRWGTMLLGMLMICSAAEQLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNATNTTSSSWETMEKGEIKNCSFNITTSIRDKVQKEYALFYNLDVVPIDNASYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSINIGPGRALYTTGEIIGDIRQAHCNISRAKWNNTLKQIVIKLREQFGNKTIVFNQSSGGDPEIVMHSFNCGGEFFYCNSTQLFTWNDTRKLNNTGRNITLPCRIKQIINMWQEVGKAMYAPPIRGQIRCSSNITGLLLTRDGGKDTNGTEIFRPGGGDMRDNWRSELYKYKVVKIEPLGVAPTKAKRRVVQREKRAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTAVPWNASWSNKSLDQIWNNMTWMEWEREIDNYTSLIYTLIEESQNQQEKNEQELLELDKWASLWNWFDITNWLWYIKIFIMIVGGLVGLRIVFAVLSIVNRVRQGYSPLSFQTRLPAPRGPDRPEGIEEEGGERDRDRSGRLVDGFLALIWVDLRSLCLFSYHRLRDLLLIVTRIVELLGRRGWEALKYWWNLLQYWSQELKNSAVSLLNATAIAVAEGTDRVIEVVQRACRAILHIPRRIRQGLERALLNLDLLKLAGDVESNPGPVWATMKWSKSSVVGWPTVRERMRRAEEPAADGVGAVSRDLEKHGAITSSNTAANNADCAWLEAQEEEEVGFPVRPQVPLRPMTYKAAVDLSHFLKEKGGLEGLIYSQKRQDILDLWVYHTQGYFPDWQNYTPGPGIRYPLTFGWCFKLVPVEPEKVEEANEGENNSLLHPMSLHGMDDPEKEVLVWKFDSRLAFHHMARELHPEYYKDC

SEQ ID NO: 54 - DNA, 5220 bp, artificial
  artificial fusion gene
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgaggtgaaggacaccaaggaggccctggagaagatcgaggaggagcagaacaagagcaagaagaaggcccagcaggccgccgccgacaccggcaacagcagccaagtgagccagaactaccccatcgtgcagaacctgcagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcctgcaccccgtgcacgccggccccatcgcccccggccagatgcgcgagccccgcggcagcgacatcgccggcaccacgagcaccctgcaggagcagatcggctggatgaccaacaacccccctatccccgtgggcgagatctacaagcgctggatcatcctgggcctgaacaagatcgtgcgcatgtacagccccacgagcatcctggacatccgccagggccccaaggagcccttccgcgactacgtggaccgcttctacaagaccctgcgggccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagaccgtgaagtgcttcaactgcgggaaggagggccacatcgccaagaactgccgcgccccccgcaagaagggctgctggaagtgcggcaaggaggggcaccagatgaaggactgcaccgagcgccaggccaacttcctgggcaagatctggcccagccacaagggccgccccggcaacttcctgcagagccgccccgagcccaccgcccctcccgaggagagcttccgcttcggcgaggagaccaccacccccagccagaagcaggagcccatcgacaaggagctgtaccccctggccagcctgcgcagcctgttcggcaacgaccccagcagccaggccatgggggccaccatggccttcttccgcgaggacctggccttcccccaaggcaaggcccgcgagttcagcagcgagcagacccgcgccaacagccccacccgccgcgagctgcaggtgtggggccgcgacaacaacagcctgagcgaggccggcgccgaccgccagggcaccgtgagcttcagcttcccccaaatcaccctgtggcagcgccccctggtgaccatcaagatcggcggccagctgaaggaggccctgctggacaccggcgccgacgacaccgtgctggaagagatgaacctgcccggccgctggaagcccaagatgatcggcggcatcggcggcttcatcaaagtgcgccagtacgaccagatcctgatcgagatctgcggccacaaggccatcggcaccgtgctcgtgggccccacccccgtgaacatcatcggccgcaacctgctgacccagatcggctgcaccctgaacttccccatcagccccatcgagaccgtgcccgtgaagctgaagcccggcatggacggccccaaggtgaagcagtggcccctgaccgaggagaagatcaaggccctggtggagatctgcaccgagatggagaaggagggcaagatcagcaagatcggccccgagaacccctacaacacccccgtgttcgccatcaagaagaaggacagcaccaagtggcgcaagctcgtggacttccgcgagctgaacaagcgcacccaggacttctgggaggtgcagctgggcatcccccaccccgccggcctgaagaagaagaagagcgtgaccgtgctggacgtgggcgacgcctacttcagcgtgcccctggacaaggacttccgcaagtacaccgccttcaccatccccagcatcaacaacgagacccccggcatccgctaccagtacaacgtgctgccccagggctggaagggcagccccgccatcttccagagcagcatgaccaagatcctggagcccttccgcaagcagaaccccgacatcgtgatctaccagtacatgaacgacctgtacgtgggcagcgacctggagatcggccagcaccgcaccaagatcgaggagctgcgccagcacctgctgcgctggggcttcaccacccccgacaagaagcaccagaaggagccccccttcctgtggatgggctacgagctgcaccccgacaagtggaccgtgcagcccatcgtgctgcccgagaaggacagctggaccgtgaacgacatccagaagctcgtgggcaagctgaactgggccagccagatctacgccggcatcaaggtgaagcagctgtgcaagctgctgcgcggcaccaaggccctgaccgaggtgatccccctgaccgaggaggccgagctggagctggccgagaaccgcgagatcctgaaggagcccgtgcacggcgtgtactacgaccccagcaaggacctgatcgccgagatccagaagcagggccagggccagtggacctaccagatctaccaggagcccttcaagaacctcaagaccggcaagtacgcccgcatgcgcggcgcccacaccaacgacgtgaagcagctgaccgaggccgtgcagaagatcgccaccgagagcatcgtgatctggggcaagacccccaagttcaagctgcccatccagaaggagacctgggagacctggtggaccgagtactggcaggccacctggatccccgagtgggagttcgtgaacacccctcccctggtgaagctgtggtatcagctggagaaggagcccatcgtgggcgccgagaccttctacgtggacggcgccgccaaccgcgagaccaagctgggcaaggccggctacgtgaccgaccgcggccgccagaaggtggtgagcctgaccgacaccaccaaccaaaagaccgagctgcaggccatccacctggccctgcaggacagcggcctggaggtgaacatcgtgaccgacagccagtacgccctgggcatcatccaggcccagcccgacaagagcgagagcgagctggtgagccagatcatcgagcagctgatcaagaaggagaaggtgtacctggcctgggtgcccgcccacaagggcatcggcggcaacgagcaggtggacaagctggtgagcgccggcatccgcaaggtgctgttcctggacggcatcgacaaggcccaggaggagcacgagaagtaccacagcaactggcgggccatggccagcgacttcaacctgccccccgtggtggccaaggagatcgtggccagctgcgacaagtgccagctgaagggcgaggccatgcacggccaggtggactgcagccccggcatctggcagctggactgcacccacctggagggcaagatcatcctggtggccgtgcacgtggccagcggctacatcgaggccgaggtgatccccgccgagaccggccaggagaccgcctacttcctgctgaagctggccggccgctggcccgtcaagaccatccacaccgacaacggcagcaacttcaccagcaccaccgtgaaggccgcctgttggtgggccggcatcaagcaggagttcggcatcccctacaacccccagagccagggcgtggtggagagcatgaacaaggagctgaagaagatcatcggccaagtgcgcgaccaggccgagcacctcaagaccgccgtgcagatggccgtgttcatccacaacttcaagcgcaagggcgggatcggcggctacagcgccggcgagcgcatcgtggacatcatcgccaccgacatccagaccaaggagctgcagaagcagatcaccaagatccagaacttccgcgtgtactaccgcgacagccgcgaccccctgtggaagggccccgccaagctgctgtggaagggcgagggcgccgtggtgatccaggacaacagcgacatcaaggtggtgccccgccgcaaggccaagatcatccgcgactacggcaagcagatggccggcgacgactgcgtggccagccgccaggacgaggaccaattgctgaacttcgacctgctgaagctggccggcgacgtggagagcaaccccggccccggatgggccaccatgaagtggagcaagagcagcgtggtgggctggcccaccgtgcgcgagcgcatgcgccgcgccgaggagcccgccgccgacggcgtgggcgccgtgagccgcgacctggagaagcacggcgccatcaccagcagcaacaccgccgccaacaacgccgactgcgcctggctggaggcccaggaggaggaggaagtgggcttccccgtgcgcccccaggtgcccctgcgccccatgacctacaaggccgccgtggacctgagccacttcctgaaggagaagggcggcctggagggcctgatctacagccagaagcgccaggacatcctggacctgtgggtgtaccacacccagggctacttccccgactggcagaactacacccccggccccggcatccgctaccccctgaccttcggctggtgcttcaagctggtgcccgtggagcccgagaaggtggaggaggccaacgagggcgagaacaacagcctgctgcaccccatgagcctgcacggcatggacgaccccgagaaggaggtgctggtgtggaagttcgacagccgcctggccttccaccacatggcccgcgagctgcaccccgagtactacaaggactgctaa

SEQ ID NO: 55 - protein, 1739 aa, artificial
  artificial fusion protein
MGARASVLSGGELDRWEKIRLRPGGKKKYKLKHIVWASRELERFAVNPGLLETSEGCRQILGQLQPSLQTGSEELRSLYNTVATLYCVHQRIEVKDTKEALEKIEEEQNKSKKKAQQAAADTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGNDPSSQAMGATMAFFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTKILEPFRKQNPDIVIYQYMNDLYVGSDLEIGQHRTKIEELRQHLLRWGFTTPDKKHQKEPPFLWMGYELHPDKWTVQPIVLPEKDSWTVNDIQKLVGKLNWASQIYAGIKVKQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWETWWTEYWQATWIPEWEFVNTPPLVKLWYQLEKEPIVGAETFYVDGAANRETKLGKAGYVTDRGRQKVVSLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKIILVAVHVASGYIEAEVIPAETGQETAYFLLKLAGRWPVKTIHTDNGSNFTSTTVKAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRDPLWKGPAKLLWKGEGAVVIQDNSDIKVVPRRKAKIIRDYGKQMAGDDCVASRQDEDQLLNFDLLKLAGDVESNPGPGWATMKWSKSSVVGWPTVRERMRRAEEPAADGVGAVSRDLEKHGAITSSNTAANNADCAWLEAQEEEEVGFPVRPQVPLRPMTYKAAVDLSHFLKEKGGLEGLIYSQKRQDILDLWVYHTQGYFPDWQNYTPGPGIRYPLTFGWCFKLVPVEPEKVEEANEGENNSLLHPMSLHGMDDPEKEVLVWKFDSRLAFHHMARELHPEYYKDC

SEQ ID NO: 56 - DNA, 7809 bp, artificial
  artificial fusion gene
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgaggtgaaggacaccaaggaggccctggagaagatcgaggaggagcagaacaagagcaagaagaaggcccagcaggccgccgccgacaccggcaacagcagccaagtgagccagaactaccccatcgtgcagaacctgcagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcctgcaccccgtgcacgccggccccatcgcccccggccagatgcgcgagccccgcggcagcgacatcgccggcaccacgagcaccctgcaggagcagatcggctggatgaccaacaacccccctatccccgtgggcgagatctacaagcgctggatcatcctgggcctgaacaagatcgtgcgcatgtacagccccacgagcatcctggacatccgccagggccccaaggagcccttccgcgactacgtggaccgcttctacaagaccctgcgggccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagaccgtgaagtgcttcaactgcgggaaggagggccacatcgccaagaactgccgcgccccccgcaagaagggctgctggaagtgcggcaaggaggggcaccagatgaaggactgcaccgagcgccaggccaacttcctgggcaagatctggcccagccacaagggccgccccggcaacttcctgcagagccgccccgagcccaccgcccctcccgaggagagcttccgcttcggcgaggagaccaccacccccagccagaagcaggagcccatcgacaaggagctgtaccccctggccagcctgcgcagcctgttcggcaacgaccccagcagccaggccatgggggccaccatggccttcttccgcgaggacctggccttcccccaaggcaaggcccgcgagttcagcagcgagcagacccgcgccaacagccccacccgccgcgagctgcaggtgtggggccgcgacaacaacagcctgagcgaggccggcgccgaccgccagggcaccgtgagcttcagcttcccccaaatcaccctgtggcagcgccccctggtgaccatcaagatcggcggccagctgaaggaggccctgctggacaccggcgccgacgacaccgtgctggaagagatgaacctgcccggccgctggaagcccaagatgatcggcggcatcggcggcttcatcaaagtgcgccagtacgaccagatcctgatcgagatctgcggccacaaggccatcggcaccgtgctcgtgggccccacccccgtgaacatcatcggccgcaacctgctgacccagatcggctgcaccctgaacttccccatcagccccatcgagaccgtgcccgtgaagctgaagcccggcatggacggccccaaggtgaagcagtggcccctgaccgaggagaagatcaaggccctggtggagatctgcaccgagatggagaaggagggcaagatcagcaagatcggccccgagaacccctacaacacccccgtgttcgccatcaagaagaaggacagcaccaagtggcgcaagctcgtggacttccgcgagctgaacaagcgcacccaggacttctgggaggtgcagctgggcatcccccaccccgccggcctgaagaagaagaagagcgtgaccgtgctggacgtgggcgacgcctacttcagcgtgcccctggacaaggacttccgcaagtacaccgccttcaccatccccagcatcaacaacgagacccccggcatccgctaccagtacaacgtgctgccccagggctggaagggcagccccgccatcttccagagcagcatgaccaagatcctggagcccttccgcaagcagaaccccgacatcgtgatctaccagtacatgaacgacctgtacgtgggcagcgacctggagatcggccagcaccgcaccaagatcgaggagctgcgccagcacctgctgcgctggggcttcaccacccccgacaagaagcaccagaaggagccccccttcctgtggatgggctacgagctgcaccccgacaagtggaccgtgcagcccatcgtgctgcccgagaaggacagctggaccgtgaacgacatccagaagctcgtgggcaagctgaactgggccagccagatctacgccggcatcaaggtgaagcagctgtgcaagctgctgcgcggcaccaaggccctgaccgaggtgatccccctgaccgaggaggccgagctggagctggccgagaaccgcgagatcctgaaggagcccgtgcacggcgtgtactacgaccccagcaaggacctgatcgccgagatccagaagcagggccagggccagtggacctaccagatctaccaggagcccttcaagaacctcaagaccggcaagtacgcccgcatgcgcggcgcccacaccaacgacgtgaagcagctgaccgaggccgtgcagaagatcgccaccgagagcatcgtgatctggggcaagacccccaagttcaagctgcccatccagaaggagacctgggagacctggtggaccgagtactggcaggccacctggatccccgagtgggagttcgtgaacacccctcccctggtgaagctgtggtatcagctggagaaggagcccatcgtgggcgccgagaccttctacgtggacggcgccgccaaccgcgagaccaagctgggcaaggccggctacgtgaccgaccgcggccgccagaaggtggtgagcctgaccgacaccaccaaccaaaagaccgagctgcaggccatccacctggccctgcaggacagcggcctggaggtgaacatcgtgaccgacagccagtacgccctgggcatcatccaggcccagcccgacaagagcgagagcgagctggtgagccagatcatcgagcagctgatcaagaaggagaaggtgtacctggcctgggtgcccgcccacaagggcatcggcggcaacgagcaggtggacaagctggtgagcgccggcatccgcaaggtgctgttcctggacggcatcgacaaggcccaggaggagcacgagaagtaccacagcaactggcgggccatggccagcgacttcaacctgccccccgtggtggccaaggagatcgtggccagctgcgacaagtgccagctgaagggcgaggccatgcacggccaggtggactgcagccccggcatctggcagctggactgcacccacctggagggcaagatcatcctggtggccgtgcacgtggccagcggctacatcgaggccgaggtgatccccgccgagaccggccaggagaccgcctacttcctgctgaagctggccggccgctggcccgtcaagaccatccacaccgacaacggcagcaacttcaccagcaccaccgtgaaggccgcctgttggtgggccggcatcaagcaggagttcggcatcccctacaacccccagagccagggcgtggtggagagcatgaacaaggagctgaagaagatcatcggccaagtgcgcgaccaggccgagcacctcaagaccgccgtgcagatggccgtgttcatccacaacttcaagcgcaagggcgggatcggcggctacagcgccggcgagcgcatcgtggacatcatcgccaccgacatccagaccaaggagctgcagaagcagatcaccaagatccagaacttccgcgtgtactaccgcgacagccgcgaccccctgtggaagggccccgccaagctgctgtggaagggcgagggcgccgtggtgatccaggacaacagcgacatcaaggtggtgccccgccgcaaggccaagatcatccgcgactacggcaagcagatggccggcgacgactgcgtggccagccgccaggacgaggaccaattgctgaacttcgacctgctgaagctggccggcgacgtggagagcaaccccggccccggatgggccaccatgcgcgtgaagggcatccgcaagaactaccagcacctgtggcgctggggcaccatgctgctgggcatgctgatgatctgcagcgccgccgagcagctgtgggtgaccgtgtactacggcgtgcccgtgtggaaggaggccaccaccaccctgttctgcgccagcgacgccaaggcctacgacaccgaggtgcacaacgtgtgggccacccacgcctgcgtgcccaccgaccccaacccccaggaggtggtgctggagaacgtgaccgagaacttcaacatgtggaagaacaacatggtggagcagatgcacgaggacatcatcagcctgtgggaccagagcctgaagccctgcgtgaagctgacccccctgtgcgtgaccctgaactgcaccgacctgcgcaacgccaccaacaccacctccagcagctgggagaccatggagaagggcgagatcaagaactgcagcttcaacatcaccacctccatccgcgacaaggtgcagaaggagtacgccctgttctacaacctggacgtggtgcccatcgacaacgccagctaccgcctgatcagctgcaacaccagcgtgatcacccaggcctgccccaaagtgagcttcgagcccatccccatccactactgcgcccccgccggcttcgccatcctgaagtgcaacgacaagaagttcaacggcaccggcccctgcaccaacgtgagcaccgtgcagtgcacccacggcatccgccccgtggtgagcacccagctgctgctgaacggcagcctggccgaggaggaggtggtgatccgcagcgagaacttcaccgacaacgccaagaccatcatcgtgcagctgaacgagagcgtggagatcaactgcacccgccccaacaacaacacccgcaagagcatcaacatcggccccggccgcgccctgtacaccaccggcgagatcatcggcgacatccgccaggcccactgcaacatcagccgcgccaagtggaacaacaccctgaagcagatcgtgatcaagctgcgcgagcagttcggcaacaagaccatcgtgttcaaccagagcagcggcggcgaccccgagatcgtgatgcacagcttcaactgcggcggcgagttcttctactgcaacagcacccagctgttcacctggaacgacacccgcaagctgaacaacaccggccgcaacatcaccctgccctgccgcatcaagcagatcatcaacatgtggcaggaagtgggcaaggccatgtacgcccctcccatccgcggccagatccgctgcagcagcaacatcaccggcctgctgctgacccgcgacggcggcaaggacaccaacggcaccgagatcttccgccccggcggcggcgacatgcgcgacaactggcgcagcgagctgtacaagtacaaggtggtgaagatcgagcccctgggcgtggcccccaccaaggccaagcgccgcgtggtgcagcgcgagaagcgggccgtgggcatcggcgccatgttcctgggcttcctgggcgccgccggcagcaccatgggcgccgccagcatgaccctgaccgtgcaggcccgccagctgctgagcggcatcgtgcagcagcagaacaacctgctgcgggccatcgaggcccagcagcacctgctgcagctgaccgtgtggggcatcaagcagctgcaggcccgcgtgctggccgtggagcgctacctgaaggaccagcagctgctgggcatctggggctgcagcggcaagctgatctgcaccaccgccgtgccctggaacgccagctggagcaacaagagcctggaccagatctggaacaacatgacctggatggagtgggagcgcgagatcgacaactacaccagcctgatctacaccctgatcgaggagagccagaaccagcaggagaagaacgagcaggagctgctggagctggacaagtgggccagcctgtggaactggttcgacatcaccaactggctgtggtacatcaagatcttcatcatgatcgtgggcggcctggtgggcctgcgcatcgtgttcgccgtgctgagcatcgtgaaccgcgtgcgccagggctacagccccctgagcttccagacccgcctgcccgccccccgcggccccgaccgccccgagggcatcgaggaggagggcggcgagcgcgaccgcgaccgcagcggccgcctggtggacggcttcctggccctgatctgggtggacctgcgcagcctgtgcctgttcagctaccaccgcctgcgcgacctgctgctgatcgtgacccgcatcgtggagctgctgggccgccgcggctgggaggccctgaagtactggtggaacctgctgcagtactggagccaggagctgaagaacagcgccgtgagcctgctgaacgccaccgccatcgccgtggccgagggcaccgaccgcgtgatcgaggtggtgcagcgggcctgccgcgccatcctgcacatcccccgccgcatccgccagggcctggagcgggccctgctgaacctcgacctgctgaagctggccggcgacgtggagagcaaccccggccccgtttgggccaccatgaagtggagcaagagcagcgtggtgggctggcccaccgtgcgcgagcgcatgcgccgcgccgaggagcccgccgccgacggcgtgggcgccgtgagccgcgacctggagaagcacggcgccatcaccagcagcaacaccgccgccaacaacgccgactgcgcctggctggaggcccaggaggaggaggaagtgggcttccccgtgcgcccccaggtgcccctgcgccccatgacctacaaggccgccgtggacctgagccacttcctgaaggagaagggcggcctggagggcctgatctacagccagaagcgccaggacatcctggacctgtgggtgtaccacacccagggctacttccccgactggcagaactacacccccggccccggcatccgctaccccctgaccttcggctggtgcttcaagctggtgcccgtggagcccgagaaggtggaggaggccaacgagggcgagaacaacagcctgctgcaccccatgagcctgcacggcatggacgaccccgagaaggaggtgctggtgtggaagttcgacagccgcctggccttccaccacatggcccgcgagctgcaccccgagtactacaaggactgctaa

SEQ ID NO: 57 - protein, 2602 aa, artificial
  artificial fusion protein
MGARASVLSGGELDRWEKIRLRPGGKKKYKLKHIVWASRELERFAVNPGLLETSEGCRQILGQLQPSLQTGSEELRSLYNTVATLYCVHQRIEVKDTKEALEKIEEEQNKSKKKAQQAAADTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGNDPSSQAMGATMAFFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTKILEPFRKQNPDIVIYQYMNDLYVGSDLEIGQHRTKIEELRQHLLRWGFTTPDKKHQKEPPFLWMGYELHPDKWTVQPIVLPEKDSWTVNDIQKLVGKLNWASQIYAGIKVKQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWETWWTEYWQATWIPEWEFVNTPPLVKLWYQLEKEPIVGAETFYVDGAANRETKLGKAGYVTDRGRQKVVSLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKIILVAVHVASGYIEAEVIPAETGQETAYFLLKLAGRWPVKTIHTDNGSNFTSTTVKAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRDPLWKGPAKLLWKGEGAVVIQDNSDIKVVPRRKAKIIRDYGKQMAGDDCVASRQDEDQLLNFDLLKLAGDVESNPGPGWATMRVKGIRKNYQHLWRWGTMLLGMLMICSAAEQLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNATNTTSSSWETMEKGEIKNCSFNITTSIRDKVQKEYALFYNLDVVPIDNASYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSINIGPGRALYTTGEIIGDIRQAHCNISRAKWNNTLKQIVIKLREQFGNKTIVFNQSSGGDPEIVMHSFNCGGEFFYCNSTQLFTWNDTRKLNNTGRNITLPCRIKQIINMWQEVGKAMYAPPIRGQIRCSSNITGLLLTRDGGKDTNGTEIFRPGGGDMRDNWRSELYKYKVVKIEPLGVAPTKAKRRVVQREKRAVGIGAMFLGFLGAAGSTMGAASMTLTVQARQLLSGIVQQQNNLLRAIEAQQHLLQLTVWGIKQLQARVLAVERYLKDQQLLGIWGCSGKLICTTAVPWNASWSNKSLDQIWNNMTWMEWEREIDNYTSLIYTLIEESQNQQEKNEQELLELDKWASLWNWFDITNWLWYIKIFIMIVGGLVGLRIVFAVLSIVNRVRQGYSPLSFQTRLPAPRGPDRPEGIEEEGGERDRDRSGRLVDGFLALIWVDLRSLCLFSYHRLRDLLLIVTRIVELLGRRGWEALKYWWNLLQYWSQELKNSAVSLLNATAIAVAEGTDRVIEVVQRACRAILHIPRRIRQGLERALLNLDLLKLAGDVESNPGPVWATMKWSKSSVVGWPTVRERMRRAEEPAADGVGAVSRDLEKHGAITSSNTAANNADCAWLEAQEEEEVGFPVRPQVPLRPMTYKAAVDLSHFLKEKGGLEGLIYSQKRQDILDLWVYHTQGYFPDWQNYTPGPGIRYPLTFGWCFKLVPVEPEKVEEANEGENNSLLHPMSLHGMDDPEKEVLVWKFDSRLAFHHMARELHPEYYKDC

SEQ ID NO: 58 - DNA, 4491 bp, artificial
  artificial fusion gene
atgggcgcccgcgccagcatcctgcgcggcggcaagctggacacctgggagaagatccgcctgcgccccggcggcaagaagcgctacatgctgaagcacctggtgtgggccagccgcgagctggagcgcttcgccctgaaccccggcctgctggagaccagcgagggctgcaagcagatcatgaagcagctgcagcccgccctgcagaccggcaccgaggagctgaagagcctgtacaacaccgtggccaccctgtactgcgtgcacgagggcatcgaggtgcgggacaccaaggaggccctggacaagatcgaggaggagcagaacaagagccagcagaaaacccagcaggccgaggccgccgacggcaaggtgtcccagaactaccccatcgtgcagaacctgcagggccagatggtgcaccaggccatcagcccccgcaccctgaacgcctgggtgaaggtgatcgaggagaaggccttcagccccgaggtgatccccatgttcaccgccctgagcgagggcgccaccccccaggacctgaacaccatgctgaacaccgtgggcggccaccaggccgccatgcagatgctgaaggacaccatcaacgaggaggccgccgagtgggaccgcctgcaccccgtgcacgccggccccgtggcccccggccagatgcgcgagccccgcggcagcgacatcgccggcaccacctccaccctgcaggagcagatcgcctggatgaccagcaacccccctatccccgtgggcgacatctacaagcgctggatcatcctgggcctgaacaagatcgtgcgcatgtacagccccgtgagcatcctggacatcaagcagggccccaaggagcccttccgcgactacgtggaccgcttcttcaagaccctgcgggccgagcaggccacccaggacgtgaagaactggatgaccgacaccctgctggtgcagaacgccaaccccgactgcaagaccatcctgcgggccctgggccccggcgccagcctggaggagatgatgaccgcctgccagggcgtgggcggccccagccacaaggcccgcgtgctggccgaggccatgagccaggccaacaacaccaacatcatgatgcagcgcagcaacttcaagggcccccgccgcatcgtgaagtgcttcaactgcggcaaggagggccacatcgcccgcaactgccgcgccccccgcaagaagggctgctggaagtgcgggaaggaggggcaccagatgaaggactgcaccgagcgccaggccaacttcctgggcaagatctggccctcccacaagggccgccccggcaacttcctgcagagccgccccgagcccaccgcccctcccgccgagagcttccgcttcgaggagaccacccccgcccccaagcaggagcccaaggaccgcgagcccctgaccagcctgaagagcctgttcggcagcgaccccctgagccaggccatgggggccaccatgttcttccgcgagaacctggccttcccgcagggcgaggcccgcgagttccccagcgagcagacccgcgccaacagccccacctcccgcgagctgcaggtgcggggcgacaacccccgcagcgaggccggcgccgagcgccagggcaccctgaacttcccgcagatcaccctgtggcagcgccccctggtgagcatcaaggtggggggccagatcaaggaggccctgctggacaccggcgccgacgacaccgtgctggaggagatcaacctgcccggcaagtggaagcccaagatgatcggcggcatcggcggcttcatcaaggtgcggcagtacgaccagatccccatcgagatctgcggcaagaaggccatcggcaccgtgctcgtgggccccacccccgtgaacatcatcggccgcaacatgctgacccagctgggctgcaccctcaacttccccatcagccccatcgagaccgtgcccgtgaagctgaagcccggcatggacggccccaaggtgaagcagtggcccctgaccgaggagaagatcaaggccctgaccgccatctgcgaggagatggagaaggagggcaagatcaccaagatcggccccgagaacccctacaacacccccgtgttcgccatcaagaagaaggacagcaccaagtggcgcaagctcgtggacttccgcgagctgaacaagcgcacccaggacttctgggaggtgcagctgggcatcccccaccccgccggcctgaagaagaagaagagcgtgaccgtgctggacgtgggcgacgcctacttcagcgtgcccctggacgaggacttccgcaagtacaccgccttcaccatccccagcatcaacaacgagacccccggcatccgctaccagtacaacgtgctgccccagggctggaagggcagccccgccatcttccagagcagcatgaccaagatcctggagcccttccgcgcccagaaccccgagatcgtgatctaccagtacatgaacgacctgtacgtgggcagcgacctggagatcggccagcaccgcgccaagatcgaggagctgcgcgagcacctgctgaagtggggcttcaccacccccgacaagaagcaccagaaggagccccccttcctgtggatgggctacgagctgcaccccgacaagtggaccgtgcagcccatccagctgcccgagaaggacagctggaccgtgaacgacatccagaagctcgtgggcaagctgaactgggccagccagatctaccccggcatcaaggtgaggcagctgtgcaagctgctgcgcggcgccaaggccctcaccgacatcgtgcccctcaccgaggaggccgagctggagctggccgagaaccgcgagatcctgaaggagcccgtgcacggcgtgtactacgaccccagcaaggacctgatcgccgagatccagaagcagggcgaccagtggacctaccagatctaccaggagcccttcaagaacctcaagaccggcaagtacgccaagatgcgcaccgcccacaccaacgacgtgaagcagctgaccgaggccgtgcagaagatcgcgatggagagcatcgtgatctggggcaagacccccaagttccgcctgcccatccagaaggagacctgggagacctggtggaccgactactggcaggccacctggatccccgagtgggagttcgtgaacacccctcccctggtgaagctgtggtatcagctggagaaggagcccatcgccggcgccgagaccttctacgtggacggcgccgccaaccgcgagaccaagatcggcaaggccggctacgtgaccgaccgcggccgccagaagatcgtgagcctgaccgagaccaccaaccagaaaaccgagctgcaggccatccagctggcgctgcaggacagcggcagcgaggtgaacatcgtgaccgacagccagtacgccctgggcatcatccaggcccagcccgacaagagcgagagcgagctggtgaaccagatcatcgagcagctgatcaagaaggagcgcgtgtacctgagctgggtgcccgcccacaagggcatcggcggcaacgagcaggtggacaagctggtgagcagcggcatccgcaaggtgctgttcctggacggcatcgacaaggcccaggaggagcacgagaagtaccacagcaactggcgggcgatggccagcgagttcaacctgccccccatcgtggccaaggagatcgtggccagctgcgacaagtgccagctgaagggcgaggccatgcacggccaggtggactgcagccccggcatctggcagctggactgcacccacctggagggcaagatcatcctggtggccgtgcacgtggccagcggctacatcgaggccgaggtgatccccgccgagaccggccaggagaccgcctacttcatcctgaagctggccggccgctggcccgtgaaggtgatccacaccgacaacggcagcaacttcaccagcgccgccgtgaaggccgcctgttggtgggccggcatccagcaggagttcggcatcccctacaacccccagagccagggcgtggtggagagcatgaacaaggagctgaagaagatcatcggccaggtgcgggaccaggccgagcacctcaagaccgccgtgcagatggccgtgttcatccacaacttcaagcgcaagggcggcatcggcgggtacagcgccggcgagcgcatcatcgacatcatcgccaccgacatccagaccaaggagctgcagaagcagatcatcaagatccagaacttccgcgtgtactaccgcgacagccgcgaccccatctggaagggccccgccaagctgctgtggaagggcgagggcgccgtggtgatccaggacaacagcgacatcaaggtggtgccccgccgcaaggccaagatcatcaaggactacggcaagcagatggccggcgccgactgcgtggccggccgccaggacgaggactaa

SEQ ID NO: 59 - protein, 1496 aa, artificial
  artificial fusion protein
MGARASILRGGKLDTWEKIRLRPGGKKRYMLKHLVWASRELERFALNPGLLETSEGCKQIMKQLQPALQTGTEELKSLYNTVATLYCVHEGIEVRDTKEALDKIEEEQNKSQQKTQQAEAADGKVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGASLEEMMTACQGVGGPSHKARVLAEAMSQANNTNIMMQRSNFKGPRRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLKSLFGSDPLSQAMGATMFFRENLAFPQGEAREFPSEQTRANSPTSRELQVRGDNPRSEAGAERQGTLNFPQITLWQRPLVSIKVGGQIKEALLDTGADDTVLEEINLPGKWKPKMIGGIGGFIKVRQYDQIPIEICGKKAIGTVLVGPTPVNIIGRNMLTQLGCTLNFPISPIETVPVKLKPGMDGPKVKQWPLTEEKIKALTAICEEMEKEGKITKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRAQNPEIVIYQYMNDLYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKEPIAGAETFYVDGAANRETKIGKAGYVTDRGRQKIVSLTETTNQKTELQAIQLALQDSGSEVNIVTDSQYALGIIQAQPDKSESELVNQIIEQLIKKERVYLSWVPAHKGIGGNEQVDKLVSSGIRKVLFLDGIDKAQEEHEKYHSNWRAMASEFNLPPIVAKEIVASCDKCQLKGEAMHGQVDCSPGIWQLDCTHLEGKIILVAVHVASGYIEAEVIPAETGQETAYFILKLAGRWPVKVIHTDNGSNFTSAAVKAACWWAGIQQEFGIPYNPQSQGVVESMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKAKIIKDYGKQMAGADCVAGRQDED

SEQ ID NO: 60 - DNA, 3102 bp, artificial
  artifical fusion gene
atgcgcgtgatgggcatccagcgcaactgccagcagtggtggatctggggcatcctgggcttctggatgctgatgatctgcaacgtgatgggcaacctgtgggtgaccgtgtactacggcgtgcccgtgtggaaggaggccaagaccaccctgttctgcgccagcgacgccaaggcctacgagaccgaggtgcacaacgtgtgggccacccacgcctgcgtgcccaccgaccccaacccccaggagatcgtgctggagaacgtgaccgagaacttcaacatgtggaagaacgacatggtggaccagatgcacgaggacatcatcagcctgtgggaccagagcctgaagccctgcgtgaagctgacccccctgtgcgtgaccctgaactgcaccaacgcggccgcgaactgcaacaccagcgccatcacccaggcctgccccaaggtgtccttcgaccccatccccatccactactgcgcccccgccggctacgccatcctgaagtgcaacaacaagaccttcaacggcaccggcccctgcaacaacgtgagcaccgtgcagtgcacccacggcatcaagcccgtggtgagcacccagctgctgctgaacggcagcctggccgaggaggagatcatcatccgcagcgagaacctgaccaacaacgccaagaccatcatcgtgcacctgaacgagagcgtggagatcgtgtgcacccgccccaacaacaacacccgcaagagcatccgcatcggccccggccagaccttctacgccaccggcgacatcatcggcgacatccgccaggcccactgcaacatcagcggcaccaagtggaacaagaccctgcagcgcgtgagcgagaagctggccgagcacttccccaacaagaccatcaagttcgcccccagcagcggcggcgacctggagatcaccacccacagcttcaactgccgcggcgagttcttctactgcaacaccagcaagctgttcaacagcacctacaacagcaacagcaccgacaacgccaacagcaccgacaactccaccatcaccctgccctgccgcatcaagcagatcatcaacatgtggcagggcgtgggccaggccatctacgcccctcccatccgcggcaacatcacctgcaagtccaacatcaccggcatcctgctgacccgcgacggcggcagcgacgccaacgagaccgagaccttccgccccggcggcggcgacatgcgcgacaactggcgcagcgagctgtacaagtacaaggtggtggagatcaagcccctgggcatcgcccccaccaaggccaagcgccgcgtggtggagcgcgagaagcgggccgtgggcatcggcgccgtgttcctgggcttcctgggcgccgccggcagcacgatgggcgccgccagcatcaccctgaccgtgcaggcccgccagctgctgagcggcatcgtgcagcagcagagcaacctgctgcgggccatcgaagcccagcagcacatgctgcagctgaccgtgtggggcatcaagcagctgcagacccgcgtgctggccatcgagcgctacctgaaggaccagcagctgctgggcatctggggctgcagcggcaagctgatctgcaccaccgccgtgccctggaacagcagctggagcaacaagagccaggccgacatctgggacagcatgacctggatgcagtgggacaaggagatcagcaactacaccggcaccatctaccgcctgctggaggagagccagaaccagcaggagaagaacgagaaggacctgctggccctggacagctggcagaacctgtggaactggttcagcatcaccaactggctgtggtacatcaagatcttcatcatgatcgtgggcggcctgatcggcctgcgcatcatcttcgccgtgctgagcatcgtgaaccgcgtgcgccagggctacagccccctgagcttccagaccctgacccccaacccccgcggccccgaccgcctgggccgcatcgaggaggagggcggcgagcaggacaaggaccgcagcatccgcctggtgagcggcttcctggccctggcctgggacgacctgcgcagcctgtgcctgttcagctaccaccgcctgcgcgacctgatcctgatcgccgcccgcgccgtggagctgctgggccgcagcagcctgcggggcctgcagcgcggctgggagaccctgaagtacctgggcagcctggtgcagtactggggcctggagctgaagaagagcgccatcagcctgctggacaccaccgccatcgccgtggccgagggcaccgaccgcatcctggagctgatccagcgcatctgccgcgccatccgcaacatcccccgccgcatccgccagggcttcgaggccgccctgcagcaattgctgaacttcgacctgctgaagctggccggcgacgtggagagcaaccccggccccgtttgggccaccatggccgccaagtggtcaaaatgtagtgtgggatggcctgctgtaagagaaagaatgcgccgcactgagccagcagcagaggaggcagcagagggagtaggagcagcatctcaagacttagataaacacggggcacttacaagcagcaacacagccgccaataatgctgattgtgcctggctggaagcgcaagaggaggaagaagaggtaggctttccagtcagacctcaggttcctttaagaccaatgacttataagggagcattcgatctcagcttctttttaaaagaaaaggggggactggaagggttaatttacagcaagaagcgccaggagatcctggacctgtgggtgtaccacacccagggcttcttccccgactggcagaactacacccccggccccggcgtgcgctaccccctgaccttcggctggtgcttcaagctggtgcccgtggaccccggcgaggtggaggaggccaacgagggcgagaacaactgcctgctgcaccccatgagccagcacggcatggaggacgaggaccgcgaggtgctgaagtggaagttcgacagccacctggcccgccgccacatggcccgcgagctgcaccccgagtactacaaggactgctaa

SEQ ID NO: 61 - protein, 1033 aa, artificial
  artificial fusion protein
MRVMGIQRNCQQWWIWGILGFWMLMICNVMGNLWVTVYYGVPVWKEAKTTLFCASDAKAYETEVHNVWATHACVPTDPNPQEIVLENVTENFNMWKNDMVDQMHEDIISLWDQSLKPCVKLTPLCVTLNCTNAAANCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISGTKWNKTLQRVSEKLAEHFPNKTIKFAPSSGGDLEITTHSFNCRGEFFYCNTSKLFNSTYNSNSTDNANSTDNSTITLPCRIKQIINMWQGVGQAIYAPPIRGNITCKSNITGILLTRDGGSDANETETFRPGGGDMRDNWRSELYKYKVVEIKPLGIAPTKAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQADIWDSMTWMQWDKEISNYTGTIYRLLEESQNQQEKNEKDLLALDSWQNLWNWFSITNWLWYIKIFIMIVGGLIGLRIIFAVLSIVNRVRQGYSPLSFQTLTPNPRGPDRLGRIEEEGGEQDKDRSIRLVSGFLALAWDDLRSLCLFSYHRLRDLILIAARAVELLGRSSLRGLQRGWETLKYLGSLVQYWGLELKKSAISLLDTTAIAVAEGTDRILELIQRICRAIRNIPRRIRQGFEAALQQLLNFDLLKLAGDVESNPGPVWATMAAKWSKCSVGWPAVRERMRRTEPAAEEAAEGVGAASQDLDKHGALTSSNTAANNADCAWLEAQEEEEEVGFPVRPQVPLRPMTYKGAFDLSFFLKEKGGLEGLIYSKKRQEILDLWVYHTQGFFPDWQNYTPGPGVRYPLTFGWCFKLVPVDPGEVEEANEGENNCLLHPMSQHGMEDEDREVLKWKFDSHLARRHMARELHPEYYKDC

SEQ ID NO: 62 - DNA, 5193 bp, artificial
  artificial fusion gene
atgggcgcccgcgccagcatcctgcgcggcggcaagctggacacctgggagaagatccgcctgcgccccggcggcaagaagcgctacatgctgaagcacctggtgtgggccagccgcgagctggagcgcttcgccctgaaccccggcctgctggagaccagcgagggctgcaagcagatcatgaagcagctgcagcccgccctgcagaccggcaccgaggagctgaagagcctgtacaacaccgtggccaccctgtactgcgtgcacgagggcatcgaggtgcgggacaccaaggaggccctggacaagatcgaggaggagcagaacaagagccagcagaaaacccagcaggccgaggccgccgacggcaaggtgtcccagaactaccccatcgtgcagaacctgcagggccagatggtgcaccaggccatcagcccccgcaccctgaacgcctgggtgaaggtgatcgaggagaaggccttcagccccgaggtgatccccatgttcaccgccctgagcgagggcgccaccccccaggacctgaacaccatgctgaacaccgtgggcggccaccaggccgccatgcagatgctgaaggacaccatcaacgaggaggccgccgagtgggaccgcctgcaccccgtgcacgccggccccgtggcccccggccagatgcgcgagccccgcggcagcgacatcgccggcaccacctccaccctgcaggagcagatcgcctggatgaccagcaacccccctatccccgtgggcgacatctacaagcgctggatcatcctgggcctgaacaagatcgtgcgcatgtacagccccgtgagcatcctggacatcaagcagggccccaaggagcccttccgcgactacgtggaccgcttcttcaagaccctgcgggccgagcaggccacccaggacgtgaagaactggatgaccgacaccctgctggtgcagaacgccaaccccgactgcaagaccatcctgcgggccctgggccccggcgccagcctggaggagatgatgaccgcctgccagggcgtgggcggccccagccacaaggcccgcgtgctggccgaggccatgagccaggccaacaacaccaacatcatgatgcagcgcagcaacttcaagggcccccgccgcatcgtgaagtgcttcaactgcggcaaggagggccacatcgcccgcaactgccgcgccccccgcaagaagggctgctggaagtgcgggaaggaggggcaccagatgaaggactgcaccgagcgccaggccaacttcctgggcaagatctggccctcccacaagggccgccccggcaacttcctgcagagccgccccgagcccaccgcccctcccgccgagagcttccgcttcgaggagaccacccccgcccccaagcaggagcccaaggaccgcgagcccctgaccagcctgaagagcctgttcggcagcgaccccctgagccaggccatgggggccaccatgttcttccgcgagaacctggccttcccgcagggcgaggcccgcgagttccccagcgagcagacccgcgccaacagccccacctcccgcgagctgcaggtgcggggcgacaacccccgcagcgaggccggcgccgagcgccagggcaccctgaacttcccgcagatcaccctgtggcagcgccccctggtgagcatcaaggtggggggccagatcaaggaggccctgctggacaccggcgccgacgacaccgtgctggaggagatcaacctgcccggcaagtggaagcccaagatgatcggcggcatcggcggcttcatcaaggtgcggcagtacgaccagatccccatcgagatctgcggcaagaaggccatcggcaccgtgctcgtgggccccacccccgtgaacatcatcggccgcaacatgctgacccagctgggctgcaccctcaacttccccatcagccccatcgagaccgtgcccgtgaagctgaagcccggcatggacggccccaaggtgaagcagtggcccctgaccgaggagaagatcaaggccctgaccgccatctgcgaggagatggagaaggagggcaagatcaccaagatcggccccgagaacccctacaacacccccgtgttcgccatcaagaagaaggacagcaccaagtggcgcaagctcgtggacttccgcgagctgaacaagcgcacccaggacttctgggaggtgcagctgggcatcccccaccccgccggcctgaagaagaagaagagcgtgaccgtgctggacgtgggcgacgcctacttcagcgtgcccctggacgaggacttccgcaagtacaccgccttcaccatccccagcatcaacaacgagacccccggcatccgctaccagtacaacgtgctgccccagggctggaagggcagccccgccatcttccagagcagcatgaccaagatcctggagcccttccgcgcccagaaccccgagatcgtgatctaccagtacatgaacgacctgtacgtgggcagcgacctggagatcggccagcaccgcgccaagatcgaggagctgcgcgagcacctgctgaagtggggcttcaccacccccgacaagaagcaccagaaggagccccccttcctgtggatgggctacgagctgcaccccgacaagtggaccgtgcagcccatccagctgcccgagaaggacagctggaccgtgaacgacatccagaagctcgtgggcaagctgaactgggccagccagatctaccccggcatcaaggtgaggcagctgtgcaagctgctgcgcggcgccaaggccctcaccgacatcgtgcccctcaccgaggaggccgagctggagctggccgagaaccgcgagatcctgaaggagcccgtgcacggcgtgtactacgaccccagcaaggacctgatcgccgagatccagaagcagggcgaccagtggacctaccagatctaccaggagcccttcaagaacctcaagaccggcaagtacgccaagatgcgcaccgcccacaccaacgacgtgaagcagctgaccgaggccgtgcagaagatcgcgatggagagcatcgtgatctggggcaagacccccaagttccgcctgcccatccagaaggagacctgggagacctggtggaccgactactggcaggccacctggatccccgagtgggagttcgtgaacacccctcccctggtgaagctgtggtatcagctggagaaggagcccatcgccggcgccgagaccttctacgtggacggcgccgccaaccgcgagaccaagatcggcaaggccggctacgtgaccgaccgcggccgccagaagatcgtgagcctgaccgagaccaccaaccagaaaaccgagctgcaggccatccagctggcgctgcaggacagcggcagcgaggtgaacatcgtgaccgacagccagtacgccctgggcatcatccaggcccagcccgacaagagcgagagcgagctggtgaaccagatcatcgagcagctgatcaagaaggagcgcgtgtacctgagctgggtgcccgcccacaagggcatcggcggcaacgagcaggtggacaagctggtgagcagcggcatccgcaaggtgctgttcctggacggcatcgacaaggcccaggaggagcacgagaagtaccacagcaactggcgggcgatggccagcgagttcaacctgccccccatcgtggccaaggagatcgtggccagctgcgacaagtgccagctgaagggcgaggccatgcacggccaggtggactgcagccccggcatctggcagctggactgcacccacctggagggcaagatcatcctggtggccgtgcacgtggccagcggctacatcgaggccgaggtgatccccgccgagaccggccaggagaccgcctacttcatcctgaagctggccggccgctggcccgtgaaggtgatccacaccgacaacggcagcaacttcaccagcgccgccgtgaaggccgcctgttggtgggccggcatccagcaggagttcggcatcccctacaacccccagagccagggcgtggtggagagcatgaacaaggagctgaagaagatcatcggccaggtgcgggaccaggccgagcacctcaagaccgccgtgcagatggccgtgttcatccacaacttcaagcgcaagggcggcatcggcgggtacagcgccggcgagcgcatcatcgacatcatcgccaccgacatccagaccaaggagctgcagaagcagatcatcaagatccagaacttccgcgtgtactaccgcgacagccgcgaccccatctggaagggccccgccaagctgctgtggaagggcgagggcgccgtggtgatccaggacaacagcgacatcaaggtggtgccccgccgcaaggccaagatcatcaaggactacggcaagcagatggccggcgccgactgcgtggccggccgccaggacgaggaccaattgctgaacttcgacctgctgaagctggccggcgacgtggagagcaaccccggccccggatgggccaccatggccgccaagtggtcaaaatgtagtgtgggatggcctgctgtaagagaaagaatgcgccgcactgagccagcagcagaggaggcagcagagggagtaggagcagcatctcaagacttagataaacacggggcacttacaagcagcaacacagccgccaataatgctgattgtgcctggctggaagcgcaagaggaggaagaagaggtaggctttccagtcagacctcaggttcctttaagaccaatgacttataagggagcattcgatctcagcttctttttaaaagaaaaggggggactggaagggttaatttacagcaagaagcgccaggagatcctggacctgtgggtgtaccacacccagggcttcttccccgactggcagaactacacccccggccccggcgtgcgctaccccctgaccttcggctggtgcttcaagctggtgcccgtggaccccggcgaggtggaggaggccaacgagggcgagaacaactgcctgctgcaccccatgagccagcacggcatggaggacgaggaccgcgaggtgctgaagtggaagttcgacagccacctggcccgccgccacatggcccgcgagctgcaccccgagtactacaaggactgctaa

SEQ ID NO: 63 - protein, 1730 aa, artificial
  artificial fusion protein
MGARASILRGGKLDTWEKIRLRPGGKKRYMLKHLVWASRELERFALNPGLLETSEGCKQIMKQLQPALQTGTEELKSLYNTVATLYCVHEGIEVRDTKEALDKIEEEQNKSQQKTQQAEAADGKVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGASLEEMMTACQGVGGPSHKARVLAEAMSQANNTNIMMQRSNFKGPRRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLKSLFGSDPLSQAMGATMFFRENLAFPQGEAREFPSEQTRANSPTSRELQVRGDNPRSEAGAERQGTLNFPQITLWQRPLVSIKVGGQIKEALLDTGADDTVLEEINLPGKWKPKMIGGIGGFIKVRQYDQIPIEICGKKAIGTVLVGPTPVNIIGRNMLTQLGCTLNFPISPIETVPVKLKPGMDGPKVKQWPLTEEKIKALTAICEEMEKEGKITKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRAQNPEIVIYQYMNDLYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKEPIAGAETFYVDGAANRETKIGKAGYVTDRGRQKIVSLTETTNQKTELQAIQLALQDSGSEVNIVTDSQYALGIIQAQPDKSESELVNQIIEQLIKKERVYLSWVPAHKGIGGNEQVDKLVSSGIRKVLFLDGIDKAQEEHEKYHSNWRAMASEFNLPPIVAKEIVASCDKCQLKGEAMHGQVDCSPGIWQLDCTHLEGKIILVAVHVASGYIEAEVIPAETGQETAYFILKLAGRWPVKVIHTDNGSNFTSAAVKAACWWAGIQQEFGIPYNPQSQGVVESMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKAKIIKDYGKQMAGADCVAGRQDEDQLLNFDLLKLAGDVESNPGPGWATMAAKWSKCSVGWPAVRERMRRTEPAAEEAAEGVGAASQDLDKHGALTSSNTAANNADCAWLEAQEEEEEVGFPVRPQVPLRPMTYKGAFDLSFFLKEKGGLEGLIYSKKRQEILDLWVYHTQGFFPDWQNYTPGPGVRYPLTFGWCFKLVPVDPGEVEEANEGENNCLLHPMSQHGMEDEDREVLKWKFDSHLARRHMARELHPEYYKDC

SEQ ID NO: 64 - DNA, 7662 bp, artificial
  artificial fusion gene
atgggcgcccgcgccagcatcctgcgcggcggcaagctggacacctgggagaagatccgcctgcgccccggcggcaagaagcgctacatgctgaagcacctggtgtgggccagccgcgagctggagcgcttcgccctgaaccccggcctgctggagaccagcgagggctgcaagcagatcatgaagcagctgcagcccgccctgcagaccggcaccgaggagctgaagagcctgtacaacaccgtggccaccctgtactgcgtgcacgagggcatcgaggtgcgggacaccaaggaggccctggacaagatcgaggaggagcagaacaagagccagcagaaaacccagcaggccgaggccgccgacggcaaggtgtcccagaactaccccatcgtgcagaacctgcagggccagatggtgcaccaggccatcagcccccgcaccctgaacgcctgggtgaaggtgatcgaggagaaggccttcagccccgaggtgatccccatgttcaccgccctgagcgagggcgccaccccccaggacctgaacaccatgctgaacaccgtgggcggccaccaggccgccatgcagatgctgaaggacaccatcaacgaggaggccgccgagtgggaccgcctgcaccccgtgcacgccggccccgtggcccccggccagatgcgcgagccccgcggcagcgacatcgccggcaccacctccaccctgcaggagcagatcgcctggatgaccagcaacccccctatccccgtgggcgacatctacaagcgctggatcatcctgggcctgaacaagatcgtgcgcatgtacagccccgtgagcatcctggacatcaagcagggccccaaggagcccttccgcgactacgtggaccgcttcttcaagaccctgcgggccgagcaggccacccaggacgtgaagaactggatgaccgacaccctgctggtgcagaacgccaaccccgactgcaagaccatcctgcgggccctgggccccggcgccagcctggaggagatgatgaccgcctgccagggcgtgggcggccccagccacaaggcccgcgtgctggccgaggccatgagccaggccaacaacaccaacatcatgatgcagcgcagcaacttcaagggcccccgccgcatcgtgaagtgcttcaactgcggcaaggagggccacatcgcccgcaactgccgcgccccccgcaagaagggctgctggaagtgcgggaaggaggggcaccagatgaaggactgcaccgagcgccaggccaacttcctgggcaagatctggccctcccacaagggccgccccggcaacttcctgcagagccgccccgagcccaccgcccctcccgccgagagcttccgcttcgaggagaccacccccgcccccaagcaggagcccaaggaccgcgagcccctgaccagcctgaagagcctgttcggcagcgaccccctgagccaggccatgggggccaccatgttcttccgcgagaacctggccttcccgcagggcgaggcccgcgagttccccagcgagcagacccgcgccaacagccccacctcccgcgagctgcaggtgcggggcgacaacccccgcagcgaggccggcgccgagcgccagggcaccctgaacttcccgcagatcaccctgtggcagcgccccctggtgagcatcaaggtggggggccagatcaaggaggccctgctggacaccggcgccgacgacaccgtgctggaggagatcaacctgcccggcaagtggaagcccaagatgatcggcggcatcggcggcttcatcaaggtgcggcagtacgaccagatccccatcgagatctgcggcaagaaggccatcggcaccgtgctcgtgggccccacccccgtgaacatcatcggccgcaacatgctgacccagctgggctgcaccctcaacttccccatcagccccatcgagaccgtgcccgtgaagctgaagcccggcatggacggccccaaggtgaagcagtggcccctgaccgaggagaagatcaaggccctgaccgccatctgcgaggagatggagaaggagggcaagatcaccaagatcggccccgagaacccctacaacacccccgtgttcgccatcaagaagaaggacagcaccaagtggcgcaagctcgtggacttccgcgagctgaacaagcgcacccaggacttctgggaggtgcagctgggcatcccccaccccgccggcctgaagaagaagaagagcgtgaccgtgctggacgtgggcgacgcctacttcagcgtgcccctggacgaggacttccgcaagtacaccgccttcaccatccccagcatcaacaacgagacccccggcatccgctaccagtacaacgtgctgccccagggctggaagggcagccccgccatcttccagagcagcatgaccaagatcctggagcccttccgcgcccagaaccccgagatcgtgatctaccagtacatgaacgacctgtacgtgggcagcgacctggagatcggccagcaccgcgccaagatcgaggagctgcgcgagcacctgctgaagtggggcttcaccacccccgacaagaagcaccagaaggagccccccttcctgtggatgggctacgagctgcaccccgacaagtggaccgtgcagcccatccagctgcccgagaaggacagctggaccgtgaacgacatccagaagctcgtgggcaagctgaactgggccagccagatctaccccggcatcaaggtgaggcagctgtgcaagctgctgcgcggcgccaaggccctcaccgacatcgtgcccctcaccgaggaggccgagctggagctggccgagaaccgcgagatcctgaaggagcccgtgcacggcgtgtactacgaccccagcaaggacctgatcgccgagatccagaagcagggcgaccagtggacctaccagatctaccaggagcccttcaagaacctcaagaccggcaagtacgccaagatgcgcaccgcccacaccaacgacgtgaagcagctgaccgaggccgtgcagaagatcgcgatggagagcatcgtgatctggggcaagacccccaagttccgcctgcccatccagaaggagacctgggagacctggtggaccgactactggcaggccacctggatccccgagtgggagttcgtgaacacccctcccctggtgaagctgtggtatcagctggagaaggagcccatcgccggcgccgagaccttctacgtggacggcgccgccaaccgcgagaccaagatcggcaaggccggctacgtgaccgaccgcggccgccagaagatcgtgagcctgaccgagaccaccaaccagaaaaccgagctgcaggccatccagctggcgctgcaggacagcggcagcgaggtgaacatcgtgaccgacagccagtacgccctgggcatcatccaggcccagcccgacaagagcgagagcgagctggtgaaccagatcatcgagcagctgatcaagaaggagcgcgtgtacctgagctgggtgcccgcccacaagggcatcggcggcaacgagcaggtggacaagctggtgagcagcggcatccgcaaggtgctgttcctggacggcatcgacaaggcccaggaggagcacgagaagtaccacagcaactggcgggcgatggccagcgagttcaacctgccccccatcgtggccaaggagatcgtggccagctgcgacaagtgccagctgaagggcgaggccatgcacggccaggtggactgcagccccggcatctggcagctggactgcacccacctggagggcaagatcatcctggtggccgtgcacgtggccagcggctacatcgaggccgaggtgatccccgccgagaccggccaggagaccgcctacttcatcctgaagctggccggccgctggcccgtgaaggtgatccacaccgacaacggcagcaacttcaccagcgccgccgtgaaggccgcctgttggtgggccggcatccagcaggagttcggcatcccctacaacccccagagccagggcgtggtggagagcatgaacaaggagctgaagaagatcatcggccaggtgcgggaccaggccgagcacctcaagaccgccgtgcagatggccgtgttcatccacaacttcaagcgcaagggcggcatcggcgggtacagcgccggcgagcgcatcatcgacatcatcgccaccgacatccagaccaaggagctgcagaagcagatcatcaagatccagaacttccgcgtgtactaccgcgacagccgcgaccccatctggaagggccccgccaagctgctgtggaagggcgagggcgccgtggtgatccaggacaacagcgacatcaaggtggtgccccgccgcaaggccaagatcatcaaggactacggcaagcagatggccggcgccgactgcgtggccggccgccaggacgaggaccaattgctgaacttcgacctgctgaagctggccggcgacgtggagagcaaccccggccccggatgggccaccatgcgcgtgatgggcatccagcgcaactgccagcagtggtggatctggggcatcctgggcttctggatgctgatgatctgcaacgtgatgggcaacctgtgggtgaccgtgtactacggcgtgcccgtgtggaaggaggccaagaccaccctgttctgcgccagcgacgccaaggcctacgagaccgaggtgcacaacgtgtgggccacccacgcctgcgtgcccaccgaccccaacccccaggagatcgtgctggagaacgtgaccgagaacttcaacatgtggaagaacgacatggtggaccagatgcacgaggacatcatcagcctgtgggaccagagcctgaagccctgcgtgaagctgacccccctgtgcgtgaccctgaactgcaccaacgcggccgcgaactgcaacaccagcgccatcacccaggcctgccccaaggtgtccttcgaccccatccccatccactactgcgcccccgccggctacgccatcctgaagtgcaacaacaagaccttcaacggcaccggcccctgcaacaacgtgagcaccgtgcagtgcacccacggcatcaagcccgtggtgagcacccagctgctgctgaacggcagcctggccgaggaggagatcatcatccgcagcgagaacctgaccaacaacgccaagaccatcatcgtgcacctgaacgagagcgtggagatcgtgtgcacccgccccaacaacaacacccgcaagagcatccgcatcggccccggccagaccttctacgccaccggcgacatcatcggcgacatccgccaggcccactgcaacatcagcggcaccaagtggaacaagaccctgcagcgcgtgagcgagaagctggccgagcacttccccaacaagaccatcaagttcgcccccagcagcggcggcgacctggagatcaccacccacagcttcaactgccgcggcgagttcttctactgcaacaccagcaagctgttcaacagcacctacaacagcaacagcaccgacaacgccaacagcaccgacaactccaccatcaccctgccctgccgcatcaagcagatcatcaacatgtggcagggcgtgggccaggccatctacgcccctcccatccgcggcaacatcacctgcaagtccaacatcaccggcatcctgctgacccgcgacggcggcagcgacgccaacgagaccgagaccttccgccccggcggcggcgacatgcgcgacaactggcgcagcgagctgtacaagtacaaggtggtggagatcaagcccctgggcatcgcccccaccaaggccaagcgccgcgtggtggagcgcgagaagcgggccgtgggcatcggcgccgtgttcctgggcttcctgggcgccgccggcagcacgatgggcgccgccagcatcaccctgaccgtgcaggcccgccagctgctgagcggcatcgtgcagcagcagagcaacctgctgcgggccatcgaagcccagcagcacatgctgcagctgaccgtgtggggcatcaagcagctgcagacccgcgtgctggccatcgagcgctacctgaaggaccagcagctgctgggcatctggggctgcagcggcaagctgatctgcaccaccgccgtgccctggaacagcagctggagcaacaagagccaggccgacatctgggacagcatgacctggatgcagtgggacaaggagatcagcaactacaccggcaccatctaccgcctgctggaggagagccagaaccagcaggagaagaacgagaaggacctgctggccctggacagctggcagaacctgtggaactggttcagcatcaccaactggctgtggtacatcaagatcttcatcatgatcgtgggcggcctgatcggcctgcgcatcatcttcgccgtgctgagcatcgtgaaccgcgtgcgccagggctacagccccctgagcttccagaccctgacccccaacccccgcggccccgaccgcctgggccgcatcgaggaggagggcggcgagcaggacaaggaccgcagcatccgcctggtgagcggcttcctggccctggcctgggacgacctgcgcagcctgtgcctgttcagctaccaccgcctgcgcgacctgatcctgatcgccgcccgcgccgtggagctgctgggccgcagcagcctgcggggcctgcagcgcggctgggagaccctgaagtacctgggcagcctggtgcagtactggggcctggagctgaagaagagcgccatcagcctgctggacaccaccgccatcgccgtggccgagggcaccgaccgcatcctggagctgatccagcgcatctgccgcgccatccgcaacatcccccgccgcatccgccagggcttcgaggccgccctgcagcaattgctgaacttcgacctgctgaagctggccggcgacgtggagagcaaccccggccccgtttgggccaccatggccgccaagtggtcaaaatgtagtgtgggatggcctgctgtaagagaaagaatgcgccgcactgagccagcagcagaggaggcagcagagggagtaggagcagcatctcaagacttagataaacacggggcacttacaagcagcaacacagccgccaataatgctgattgtgcctggctggaagcgcaagaggaggaagaagaggtaggctttccagtcagacctcaggttcctttaagaccaatgacttataagggagcattcgatctcagcttctttttaaaagaaaaggggggactggaagggttaatttacagcaagaagcgccaggagatcctggacctgtgggtgtaccacacccagggcttcttccccgactggcagaactacacccccggccccggcgtgcgctaccccctgaccttcggctggtgcttcaagctggtgcccgtggaccccggcgaggtggaggaggccaacgagggcgagaacaactgcctgctgcaccccatgagccagcacggcatggaggacgaggaccgcgaggtgctgaagtggaagttcgacagccacctggcccgccgccacatggcccgcgagctgcaccccgagtactacaaggactgctaa

SEQ ID NO: 65 - protein, 2553 aa, artificial
  artificial fusion protein
MGARASILRGGKLDTWEKIRLRPGGKKRYMLKHLVWASRELERFALNPGLLETSEGCKQIMKQLQPALQTGTEELKSLYNTVATLYCVHEGIEVRDTKEALDKIEEEQNKSQQKTQQAEAADGKVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGASLEEMMTACQGVGGPSHKARVLAEAMSQANNTNIMMQRSNFKGPRRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLKSLFGSDPLSQAMGATMFFRENLAFPQGEAREFPSEQTRANSPTSRELQVRGDNPRSEAGAERQGTLNFPQITLWQRPLVSIKVGGQIKEALLDTGADDTVLEEINLPGKWKPKMIGGIGGFIKVRQYDQIPIEICGKKAIGTVLVGPTPVNIIGRNMLTQLGCTLNFPISPIETVPVKLKPGMDGPKVKQWPLTEEKIKALTAICEEMEKEGKITKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRAQNPEIVIYQYMNDLYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKEPIAGAETFYVDGAANRETKIGKAGYVTDRGRQKIVSLTETTNQKTELQAIQLALQDSGSEVNIVTDSQYALGIIQAQPDKSESELVNQIIEQLIKKERVYLSWVPAHKGIGGNEQVDKLVSSGIRKVLFLDGIDKAQEEHEKYHSNWRAMASEFNLPPIVAKEIVASCDKCQLKGEAMHGQVDCSPGIWQLDCTHLEGKIILVAVHVASGYIEAEVIPAETGQETAYFILKLAGRWPVKVIHTDNGSNFTSAAVKAACWWAGIQQEFGIPYNPQSQGVVESMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKAKIIKDYGKQMAGADCVAGRQDEDQLLNFDLLKLAGDVESNPGPGWATMRVMGIQRNCQQWWIWGILGFWMLMICNVMGNLWVTVYYGVPVWKEAKTTLFCASDAKAYETEVHNVWATHACVPTDPNPQEIVLENVTENFNMWKNDMVDQMHEDIISLWDQSLKPCVKLTPLCVTLNCTNAAANCNTSAITQACPKVSFDPIPIHYCAPAGYAILKCNNKTFNGTGPCNNVSTVQCTHGIKPVVSTQLLLNGSLAEEEIIIRSENLTNNAKTIIVHLNESVEIVCTRPNNNTRKSIRIGPGQTFYATGDIIGDIRQAHCNISGTKWNKTLQRVSEKLAEHFPNKTIKFAPSSGGDLEITTHSFNCRGEFFYCNTSKLFNSTYNSNSTDNANSTDNSTITLPCRIKQIINMWQGVGQAIYAPPIRGNITCKSNITGILLTRDGGSDANETETFRPGGGDMRDNWRSELYKYKVVEIKPLGIAPTKAKRRVVEREKRAVGIGAVFLGFLGAAGSTMGAASITLTVQARQLLSGIVQQQSNLLRAIEAQQHMLQLTVWGIKQLQTRVLAIERYLKDQQLLGIWGCSGKLICTTAVPWNSSWSNKSQADIWDSMTWMQWDKEISNYTGTIYRLLEESQNQQEKNEKDLLALDSWQNLWNWFSITNWLWYIKIFIMIVGGLIGLRIIFAVLSIVNRVRQGYSPLSFQTLTPNPRGPDRLGRIEEEGGEQDKDRSIRLVSGFLALAWDDLRSLCLFSYHRLRDLILIAARAVELLGRSSLRGLQRGWETLKYLGSLVQYWGLELKKSAISLLDTTAIAVAEGTDRILELIQRICRAIRNIPRRIRQGFEAALQQLLNFDLLKLAGDVESNPGPVWATMAAKWSKCSVGWPAVRERMRRTEPAAEEAAEGVGAASQDLDKHGALTSSNTAANNADCAWLEAQEEEEEVGFPVRPQVPLRPMTYKGAFDLSFFLKEKGGLEGLIYSKKRQEILDLWVYHTQGFFPDWQNYTPGPGVRYPLTFGWCFKLVPVDPGEVEEANEGENNCLLHPMSQHGMEDEDREVLKWKFDSHLARRHMARELHPEYYKDC

SEQ ID NO: 66 - DNA, 7 bp, artificial
  artificial sequence
  misc_feature (6)..(6): n is a, c, g, or t
tttttnt

SEQ ID NO: 67 - DNA, 32 bp, artificial
  artificial sequence
tctcgagctcaatgaattcagtgactgtatca

SEQ ID NO: 68 - DNA, 33 bp, artificial
  artificial sequence
cgcggtaccgtcttaataaataaacccttgagc

SEQ ID NO: 69 - protein, 37 aa, artificial
  artificial sequence
AAAGCTTAGATCTGCCACCATGAATTCAGTGACTGTA

SEQ ID NO: 70 - DNA, 33 bp, artificial
  artificial sequence
agcggccgctacgtattaataaataaacccttg

SEQ ID NO: 71 - DNA, 36 bp, artificial
  artificial sequence
aagcttagatctgccaccatgtccaatttactgacc

SEQ ID NO: 72 - DNA, 73 bp, artificial
  artificial sequence
gtttaaacgcggccgcctattctagtgttagtgatgctagtggtgatggtagtgttacatcgccatcttccag

SEQ ID NO: 73 - protein, 13 aa, artificial
  artificial sequence
VTLPSPLASLTLE

(19) United States
(12) Patent Application Publication
(10) Pub. No.: US 2007/0275010 A1
(43) Pub. Date: Nov. 29, 2007

(54) Mva Vaccines

(76) Inventors: Mark Feinberg, Philadelphia, PA (US); David Garber, Atlanta, GA (US)

Correspondence Address: Thomas, Kayden, Horstemeyer & Risley, 100 Galleria Parkway, Suite 1750, Atlanta, GA, 30339, US

(21) Appl. No.: 10/572,229

(22) PCT Filed: Sep. 20, 2004
(86) PCT No.: PCT/US04/30849

§ 371 Date: Dec. 1, 2006

Related U.S. Application Data
(60) Provisional application No. 60/504,030, filed on Sep. 18, 2003.

(51) Int. Cl. A61K 39/12; A61P 31/12; A61P 31/18; C12N 15/63; C12N 15/86; C12N 5/10; C12N 7/00

(52) U.S. Cl. 424/199.1; 435/235.1; 435/320.1; 435/349; 435/456

(57) ABSTRACT

Recombinant modified vaccinia Ankara vectors are provided having a null mutation in a gene necessary for replication of the recombinant modified vaccinia Ankara virus and at least one heterologous antigen. The disclosed vectors optionally encode at least one pro-apoptotic factor, at least one anti-apoptotic factor, at least one immunomodulator, and combinations thereof. Cells complementing the null mutation the disclosed vectors are also provided.

### CROSS-REFERENCE TO RELATED APPLICATION

This application claims benefit of and priority to U.S. provisional patent application No. 60/504,030 filed on Sep. 18, 2003, and where permissible, is incorporated by reference in its entirety.

### STATEMENT CONCERNING FEDERALLY SPONSORED RESEARCH OR DEVELOPMENT

The work described herein was supported, in part, by Grant No. P01-A146007 awarded by the National Institutes of Health. Accordingly, the U.S. government has certain rights in the claimed subject matter.

### BACKGROUND

1. Technical Field

In general, aspects of the present disclosure are directed to methods and compositions relating to vaccines, in particular, viral vectors capable of eliciting an immune response.

2. Related Art

Infectious diseases including AIDS, malaria, tuberculosis and hepatitis C remain significant health threats throughout the world. In 2003, it is estimated that approximately 40 million people worldwide are living with HIV, and approximately 5 million people will be newly infected. A majority of these new antigens occur in developing nations that lack the economic resources and infrastructure to acquire and successfully deliver effective antiviral therapy. In the absence of effective therapy, the vast majority of these individuals will die of AIDS-a fate suffered by over three million people last year alone. Development of an effective HIV vaccine represents the single best hope for curtailing the suffering and devastation wrought by the AIDS pandemic. A major challenge in the development of live vaccines and immunotherapy vectors is the generation of safe delivery systems for clinical use that induce robust immune responses. Poxviruses (including canarypox, vaccinia, and fowlpox) are the most common live-vector HIV vaccine candidates. Poxviruses are capable of accommodating large amounts of foreign genes (heterologous DNA) and can infect mammalian cells, resulting in expression of a large amount of foreign protein. Modified Vaccinia Ankara (MVA) is an attenuated strain of vaccinia virus that infects, but is unable to replicate completely in human cells. MVA is avirulent in animal models of immunodeficiency and was safely administered, without significant side effects, to approximately 120,000 persons (including many individuals at high risk for complications of standard W vaccination) in the final stages of the smallpox eradication campaign (Moss, B., Genetically engineered poxviruses for recombinant gene expression, vaccination, and safety. Proceedings of the National Academy of Sciences of the United States of America, 1996. 93(21): p. 11341-8; Mayr, A. V., et al., Abstammung, eigenschaften und verwendung des attenuierten vaccinia-stammes MVA. Infection, 1975. 3: p. 6-14; Hochstein-Mintzel, V., et al, Vaccinia- und variolaprotektive Wirkung des modifizierten Vaccinia-Stammes MVA bei intramuskularer Immunisierung. Zentralblatt Fur Bakteriologie, Parasitenkunde, Infektionskrankheiten Und Hygiene—Erste Abteilung Originate—Reihe A: Medizinische Mikrobiologie Und Parasitologie, 1975. 230(3): p. 283-97; Mahnek, H. and A. Mayr, Erfahrungen bel der schutzimpfung gegen orthopocken von mensch und Hermit dem impfstamm MVA. Berl Munch. Tierartz. Wschr., 1994.107: p. 253-256; Stickl, H., et al., MVA-stufenimpfung gegen pocken. Kleinische erprobung des attenuierten pocken-lebendimpfstoffes, stamm MVA. Dtsch. Med. Wrsch., 1974. 99: p. 2386-2392; Mayr, A,, et al., Der Pockenimpfstamm MVA: Marker, genetische Struktur, Erfahrungen mit der parenteralen Schutzimpfung und Verhalten im abwehrgeschwachten Organismus. Zentralblatt Fur Bakteriologie, Parasitenkunde, Infektionskrankheiten Und Hygiene—Erste Abteilung Originale—Reihe B: Hygiene, Betriebshygiene, Preventive Medizinf 1978.167(5-6): p. 375-90). MVA was derived by prolonged passage on chicken embryo fibroblasts (CEFs) and accumulated sizeable deletions (approximately 30 kilobases [or 15%]) of the coding capacity of the parental vaccinia virus (W) genome (Mayr, A., V. Hochstein-Mintzel, and H, Stickl, Abstammung, eigenschaften und verwendung des attenuierten vaccinia-stammes MVA. Infection, 1975.3: p. 6-14; Mahnek, H. and A. Mayr, Erfahrungen bel der schutzimpfung gegen orthopocken von mensch und Hermit dem impfstamm MVA. Berl Munch. Tierartz. Wschr., 1994. 107: p. 253-256; Stickl, H., et al., MVA-stufenimpfung gegen pocken. Kleinische erprobung des aftenuierten pocken-lebendimpfstoffes, stamm MVA. Dtsch. Med. Wrsch,, 1974. 99: p. 2386-2392; Mayr, A,, et al., Der Pockenimpfstamm MVA: Marker, genetische Struktur, Erfahrungen mit der parenteralen Schutzimpfung und Verhalten im abwehrgeschwachten Organismus. Zentralblaft Fur Bakteriologie, Parasitenkunde, lnfektionskrankheiten Und Hygiene—Erste Abteilung Originale—Reihe B: Hygiene, Betriebshygiene, Preventive Medizinf 1978.167(5-6): p. 375-90; Antoine, G._{f} et al., The complete genomic sequence of the modified vaccinia Ankara strain: comparison with other orthopoxviruses. Virology, 1998. 244(2): p. 365-96; Blanchard, T. J., et al, Modified vaccinia virus Ankara undergoes limited replication in human cells and lacks several immunomodulatory proteins: implications for use as a human vaccine. Journal of General Virology, 1998. 79(Pt5):p. 1159-67). Deletion of genes, including those that effect viral host range, have resulted in the block to MVA replicating productively in human cells. The replication block in such ‘non-permissive’ cells occurs at a very late stage in the MVA life cycle after expression of both early (E) and late (L) W gene products (Blanchard, T. J., et al., Modified vaccinia virus Ankara undergoes limited replication in human cells and lacks several immunomodulatory proteins: implications for use as a human vaccine. Journal of General Virology, 1998. 79(Pt5):p. 1159-67; Carroll, M. W. and B. Moss, Host range and cytopathogenicity of the highly attenuated MVA strain of vaccinia virus: propagation and generation of recombinant viruses in a nonhuman mammalian cell line. Virology, 1997. 238(2): p.198-211) (FIG. 1). The inability of MVA to undergo >1 infection cycle in a human host is an inherent safety feature of MVA-based vaccines. Although some mammalian cells can propagate MVA, passaging of MVA in mammalian cells, however, presumably also increases virulence in mammals, resulting in new MVA-like strains with unknown safety profiles in humans.

The MVA genome has a number of gene deletions that include many, but not all, of the genes associated in poxvirus evasion of host immune responses. Exemplary deleted genes include the soluble receptors for IFN-γ, IFN-α/β, tumor necrosis factor and CC-chemokines (Antoine, G._{f} et al., The complete genomic sequence of the modified vaccinia Ankara strain: comparison with other orthopoxviruses. Virology, 1998. 244(2): p. 365-96; Blanchard, T. J., et al., Modified vaccinia virus Ankara undergoes limited replication in human cells and lacks several immunomodulatory proteins: implications for use as a human vaccine. Journal of General Virology, 1998. 79(Pt 5):p. 1159-67), which may contribute to MVA's favorable immunogenicity when used to express heterologous antigens in prime-boost regimens (e.g., DNA-primed/MVA-boosted HIV vaccines (Robinson, H. L., New hope for an AIDS vaccine. Nat Rev Immunol, 2002. 2(4): p. 239-50)). Further, although MVA-based vectors can substantially boost immune responses primed by other vaccine modalities (e.g., DNA), MVA appears to be relatively impaired compared to standard VV strains, in its ability to raise broad anti-W CD4+ and CD8+ cellular immune responses. As a vaccine vector, MVA has been shown to elicit immurie responses in animals against a number of heterologous viral antigens (including HIV) (Moss, B., Genetically engineered poxviruses for recombinant gene expression, vaccination, and safety. Proceedings of the National Academy of Sciences of the United States of America, 1996. 93(21): p. 11341-8).

Despite these advantages, there are several limitations to the use of currently available recombinant MVA vectors as vaccines against HIV infection. These include the need for complex vaccination regimens (multiple DNA primings, followed by MVA “boosting” immunizations) to achieve robust cellular immune responses and the inability to effectively “boost” immune responses against heterologous antigens by repeated immunizations with the same recombinant MVA vector (as a result of elicitation of anti-vaccinia neutralizing antibody responses) (Hanke, T., et al., Enhancement of MHC class I-restricted peptide-specific T cell induction by a DNA prime/MVA boost vaccination regime. Vaccine, 1998.16(5): p.439-45; Seth, A., et al., Immunization with a modified vaccinia virus expressing simian immunodeficiency virus (SIV) Gag-Pol primes for an anamnestic Gag-specific cytotoxic T-lymphocyte response and is associated with reduction of viremia after SIV challenge. J Virol, 2000. 74(6): p. 2502-9; Seth, A., et al., Recombinant modified vaccinia virus Ankara-simian immunodeficiency virus gag pol elicits cytotoxic T lymphocytes in rhesus monkeys detected by a major histocompatibility complex class I/peptide tetramer. Proceedings of the National Academy of Sciences of the United States of America, 1998. 95(17): p.10112-6; Amara, R. R., et al., Control of a mucosal challenge and prevention of AIDS by a multiprotein DNA/MVA vaccine. Science, 2001. 292(5514): p. 69-74). Enhancing the immunogenicity of a MVA vector may result in effective single-inoculation immunizations. This would lead directly to successful immunization of greater numbers of people, due to reduced costs of vaccine production and to increased ease of vaccine administration. Alternatively, effective boosting of anti-HIV immune responses through subsequent immunizations may be required to afford durable protection against HIV/AIDS (even for vaccine vectors that exhibit enhanced immunogenic potential). The use of the identical vaccine for initial immunization and subsequent booster immunizations would be advantageous over serial vaccinations with several different vaccine modalities by reducing the complexity and ensuing costs of vaccine production. The growth of MVA in mammalian cells (baby hamster kidney cells) has been described opening the way to also produce MVA vectors in permanent cells. Unfortunately, these mammalian cells are transformed cells, and transformed cells are unsuitable for the large scale manufacture of viral vaccines because of the uncertainty the transformation may have on the viral vaccine. Production of viral vaccines in transformed cells may confer undesirable attributes to the viral vaccine. Accordingly, there is a need for improved MVA vaccines and methods of producing them.

### SUMMARY

Recombinant modified vaccinia Ankara virus (rMVA) vectors and methods of producing and using them are provided. The disclosed rMVA vectors are useful as vaccines for vaccinating a host, such as a mammal, against a pathology such as an infectious disease or cancer. Representative vaccines are directed toward treating or preventing viral diseases such as HIV, hepatitis, and smallpox, among others.

One aspect provides a recombinant modified vaccinia Ankara having a null mutation in a vaccinia gene necessary for replication of the recombinant modified vaccinia Ankara virus. An exemplary gene necessary for replication is the vaccinia uracil DNA glycosylase gene. In another aspect, the rMVA includes a heterologous nucleic acid sequence encoding an antigen or a fragment of the antigen. The antigen can be selected from any number of viral, animal, plant, nematode, plasmodium, or bacterial polypeptides or polynucleotides. The disclosed rMVAs can be propagated in a complementing cell line. An exemplary complementing cell line can be an avian fibroblast cell engineered to express vaccinia uracil DNA glycosylase.

Accordingly, another aspect provides a system for producing recombinant modified vaccinia Ankara virus. The system includes an immortalized, non-transformed avian cell, for example, a fibroblast cell, infected with a recombinant modified vaccinia Ankara virus. The recombinant modified vaccinia Ankara virus generally includes a first null mutation in a vaccinia gene necessary for replication of the recombinant modified vaccinia Ankara virus. The avian cell is engineered to express the vaccinia gene necessary for viral replication to enable the recombinant modified vaccinia Ankara virus to replicate in the cell.

Other aspects provide rMVAs that include a null mutation in a vaccinia gene necessary for replication in a non-complementing cell, and from one to four heterologous nucleic acid sequences. The heterologous nucleic acid sequences can encode one or more antigens from one or more viruses or organisms. Alternatively, the heterologous nucleic acid sequences can independently and alternatively encode combinations of an antigen, pro-apoptotic factor, anti-apoptotic factor, and an immunomodulator. It will be appreciated that multiples of any one class of these polypeptides can be included. For example, representative rMVAs encode at least one antigen, and optionally, at least one pro-apoptotic factor, optionally at least one anti-apoptotic factor, optionally at least one immunomodulator, and combinations thereof. Additionally, the disclosed rMVAs can include additional null mutations in vaccinia genes to minimize an immune response to vaccinia antigens. Additional vaccinia genes that can contain null mutations include, but are not limited to, IL1 beta receptor, A46R, IL-18BP, A41 L, and E3L.

Still another aspect provides new vaccine vectors, for example vaccine vectors that simultaneously express consensus antigens that are encoded by several HIV genes (gag, pol, env, nef) that represent multiple independent subtypes of HIV-1 (clades B, C, A) or fusion proteins thereof.

Yet another aspect provides an immortalized, non-transformed avian fibroblast cell infected with the disclosed modified -vaccinia Ankara virus vectors. A representative cell includes a DF-1 cell and derivatives thereof. Derivatives of DF-1 cells include DF-1 cells that are engineered to complement a null mutation in the disclosed rMVA vectors. For example, one aspect of the present disclosure provides an immortalized, non-transformed avian fibroblast cell engineered to constitutively express vaccinia uracil DNA glycosylase.

Another aspect provides a method of propagating a modified vaccinia Ankara virus by culturing an immortalized, non-transformed cell engineered to express modified Ankara virus uracil DNA glycosylase; and infecting the cell with a recombinant modified vaccinia Ankara virus, wherein the recombinant modified vaccinia Ankara virus cannot express functional uracil DNA glycosylase. The rMVA can then be plaque purified from a plurality of infected cells.

Still another aspect provides a method of vaccinating a host by administering the disclosed vectors and compositions to the host, for example in an amount sufficient to affect an immune response in the host.

Another aspect provides a modified vaccinia Ankara virus comprising a nucleic acid encoding a pro-apoptotic factor, an anti-apoptotic factor, an immunomodulator, a heterologous antigen, fragments thereof or combinations thereof. The rMVAs can also have at least one null mutation in a gene necessary for rMVA replication or a gene selected from the group consisting of IL1 beta receptor, A46R, IL-18BP, A41L, and E3L.

Another aspect provides a smallpox vaccine comprising a null mutation in a gene necessary for replication of the recombinant modified vaccinia Ankara virus, and one or more nucleic acid sequences operably linked to an early stage viral promoter, wherein the one more nucleic acid sequences encode one or more genes selected from the group consisting of B5R, A33R, L1R, A27L, and fragments thereof.

The disclosed rMVAs are advantageously propagated in a non-mammalian, non-transformed cell. Thus, the disclosed rMVAs and methods of producing them are suitable for large scale manufacture according to Good Manufacturing Practices for pharmaceuticals and biologics.

### BRIEF DESCRIPTION OF THE DRAWINGS

FIG. 1 is a diagram showing that MVA infection of non-permissive cells is blocked at a late stage.

FIGS. 2A and B are graphs showing that DF-1 cells support high-level growth of MVA.

FIG. 3 is a diagram showing an exemplary structure of MVA transfer vectors.

FIG. 4 is a graph showing that zeocin inhibits MVA growth in DF-1 cells.

FIG. 5A is a diagram of an exemplary recombinant virus MVA-GZ that encodes GFP-Zeocin fusion protein under the control of early modified H5 promoter (E).

FIG. 5B is a diagram showing an exemplary transfection-infection method of generating representative MVA recombinants.

FIG. 6A is a diagram of an exemplary expression vector encoding UDG for generation of DF-1 derived cell lines that stably express UDG.

FIG. 6B is a table showing cell lines that were stably transfected with udg-expression vectors and that complemented udg^{ts} W mutants.

FIG. 7 is a diagram showing that MVAAUDG infection of non-complementing cells is blocked at the stage of viral DNA replication, but may be propagated in DF-1 derived cell lines that express UDG in trans.

FIG. 8A is a diagram showing an exemplary udg-deletion MVA recombinant virus.

FIG. 8B is a Southern Blot confirming udg-deletion of an exemplary MVA recombinant virus.

FIG. 9A is a graph showing that MVAAUDG recombinants grow on DF-1 complementing cells.

FIG. 9B is a graph showing that MVAAUDG recombinants do not grow on non-complementing DF-1 cells.

FIG. 10 is a graph showing viral DNA replication is blocked during MVAAUDG infection of non-complementing DF-1 cells.

FIGS. 11A and 11B are graphs showing the activation of pro-apoptotic proteases in DF-1 non-complementing cells and DF-1 complementing cells respectively.

FIGS. 12A and 12B are graphs showing MVAAUDG-GAG elicits higher levels of CD8+ T-cell proliferation responses that does MVA(udg+)-GAG (FIG. 12B) following immunization of rhesus macaques.

FIG. 13A is a diagram showing Cre-mediated excision of loxP-flanked gfp-zeo expression cassette from the MVA genome.

FIG. 13B is a chart showing the percent recombination using Cre-mediated excision of gfp-zeo.

FIG. 14A is a panel of graphs showing the percentage of cells or each lineage of murine splenocytes infected with MVA-GFP.

FIG. 14B is a panel of graphs showing the percentage of total infected spelenocytes of each lineage of murine splenocytes infected with MVA-GFP.

FIG. 15 is a graph showing the time course of MVA gene experssion and apoptosis in murine BMDCs.

FIG. 16A is a panel of graphs showing murine BMDCs and DF-1 cells mock-treated or infected with either rMVA-GFP or rVV-EGFP.

FIG. 16B is a panel of graphs showing human monocyte-derived DCs infeceted with VV-GFP or MVA-GFP and early and late gene expression.

FIG. 17A is a graph showing flow cytometry detection of NP antigen-specific CD8+ splenocytes via tetramer staining.

FIG. 17B is a graph showing antigen-specific production of IFN-γ in response to in vitro stimulation with NP peptide.

FIGS. 18A and 18B are graphs showing quantitation of p24Gag and gp120Env expression from synthetic consensus HIV-1 genes and gene fusions.

FIG. 19 is a diagram of exemplary MVA-based vaccine vectors that encode HIV-1 consensus genes.

FIG. 20 is a diagram of an exemplary multivalent MVA-based vaccine that encodes HIV-1 consensus genes.

FIG. 21 is a graphs showing BcIXL inhibition of MVA-induced apoptosis in human dendritic cells.

FIG. 22 is a panel of fluorescent micrographs showing MVAΔudg does not exhibit DNA replication during infection of non-complementing cells.

FIG. 23 is an autoradiograph of a gel showing MVAΔudg does not express viral late genes during infection of non-complementing cells.

FIG. 24 is a graph showing HIV-Gag expression is comparable during infection with MVAΔudg-gag and MVA-gag.

FIG. 25 is a panel of graphs showing MVAΔudg-gag induces greater CD8+ and CD4+ T-cell proliferation responses in vivo than does MVA-gag.

FIG. 26 are plots showing MVAΔudg-gag is a significantly better priming vector than MVA-gag in rhesus macaques.

FIG. 27 is a graph showing MVAΔudg-gag elicits higher levels of Gag-specific cellular memory immune responses than MVA(udg+)-gag in macaques.

FIG. 28 is a panel of plots showing MVAΔudg-gag elicits higher levels of cellular immune response that MVA(udg+)-gag following single-dose immunization of rhesus macaques.

FIG. 29 is an autoradiograph of a gel showing expression of HIV subtype B consensus antigens.

FIGS. 30A and 30B are photomicrographs showing formation of virus-like particles or cytoplasmic protein aggregates in 293 cells infected with plasmids that express full length gag (29A) or gag-pol fusion protein (29B).

FIG. 31 is a diagram showing promotion of cross-presentation is an exemplary strategy for enhancing immunogenicity of the disclosed MVA vectors.

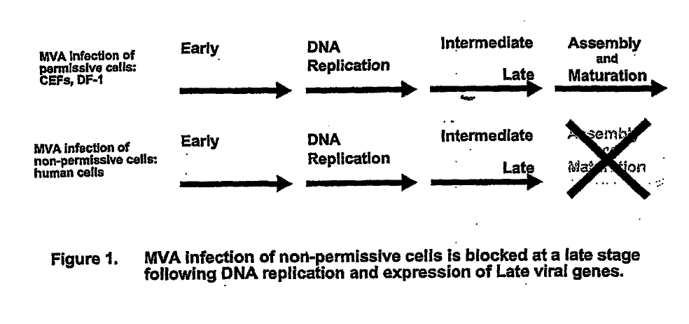

### DETAILED DESCRIPTION

1. Definitions

The term “organism” refers to any living entity comprised of at least one cell. A living organism can be as simple as, for example, a single eukaryotic cell or as complex as a mammal, including a human being.

The term “therapeutically effective amount” as used herein refers to that amount of the compound being administered which will relieve to some extent one or more of the symptoms of the disorder being treated. In reference to vascular pathologies or conditions, a therapeutically effective amount refers to that amount which has the effect of (1) reducing inflammation, plaque formation, or monocyte adhesion, (2) inhibiting (that is, slowing to some extent, preferably stopping) inflammation, plaque formation, or monocyte adhesion (3) relieving to some extent (or, preferably, eliminating) one or more symptoms associated with vascular inflammation including but not limited to atherosclerosis and other vascular inflammation pathologies.

“Pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, or a pharmaceutically acceptable salts thereof, with other chemical components, such as physiologically acceptable carriers and excipients. The purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to inflammation, these terms simply mean that the life expectancy of an individual affected with an inflammation pathology will be increased or that one or more of the symptoms of the disease will be reduced.

The term “prodrug” refers to an agent, including nucleic acids and proteins, which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N. J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11,:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p.185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

The term “nucleic acid” is a term of art that refers to a string of at least two base-sugar-phosphate combinations. For naked DNA delivery, a polynucleotide contains more than 120 monomeric units since it must be distinguished from an oligonucleotide. However, for purposes of delivering RNA, RNAi and siRNA, either single or double stranded, a polynucleotide contains 2 or more monomeric units. Nucleotides are the monomeric units of nucleic acid polymers. The term includes deoxyribonucleic acid (DNA) and ribonucleic acid (RNA). RNA may be in the form of an tRNA (transfer RNA), snRNA (small nuclear RNA), rRNA (ribosomal RNA), mRNA (messenger RNA), anti-sense RNA, RNAi, siRNA, and ribozymes. The term also includes PNAs (peptide nucleic acids), phosphorothioates, and other variants of the phosphate backbone of native nucleic acids. Anti-sense is a polynucleotide that interferes with the function of DNA and/or RNA. Natural nucleic acids have a phosphate backbone, artificial nucleic acids may contain other types of backbones, but contain the same bases.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gin, His), (Asp: Glu, Cys, Ser), (Gin: Asn), (Glu: Asp), (Gly: Ala), (His: Asn, Gin), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Lesk, A. M., Ed. (1988) Computational Molecular Biology, Oxford University Press, New York; Smith, D. W., Ed. (1993) Biocomputing: Infomatics and Genome Projects. Academic Press, New York; Griffin, A. M., and Griffin, H. G., Eds. (1994) Computer Analysis of Sequence Data: Part I, Humana Press, New Jersey; von Heinje, G. (1987) Sequence Analysis in Molecular Biology, Academic Press; Gribskov, M. and Devereux, J., Eds. (1991) Sequence Analysis Primer. M Stockton Press, New York; Carillo, H. and Lipman, D. (1988) SIAM J Applied Math., 48,1073).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, ((1970) J. Mol. Biol., 48, 443-453) algorithm (e.g., NBLAST, and XBLAST).

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “purified” and like terms relate to the isolation of a molecule or compound in a form that is substantially free (at least 60% free, preferably 75% free, and most preferably 90% free) from other components normally associated with the molecule or compound in a native environment

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “treating” includes alleviating the symptoms associated with a specific disorder or condition and/or preventing or eliminating said symptoms.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “exogenous DNA” or “exogenous nucleic acid sequence” or “exogenous polynucleotide” refers to a nucleic acid sequence that was introduced into a cell or organelle from an external source. Typically the introduced exogenous sequence is a recombinant sequence.

As used herein, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. The nucleic acid may be in the form of naked DNA or RNA, associated with various proteins or the nucleic acid may be incorporated into a vector.

As used herein, the term “vector” is used in reference to a vehicle used to introduce a nucleic acid sequence into a cell. A viral vector is virus that has been modified to allow recombinant DNA sequences to be introduced into host cells or cell organelles.

The term “primary cell” means a cell or cell line taken directly from a living organism, which is not immortalized.

The term “immortalized cell” means a cell that is able to grow and reproduce without restriction (given ample nutrients). Immortalized cells exhibit contact inhibition and will grow as monolayers in culture. Additionally, immortalized cells will not produce tumors when injected into immunocompromised mice.

The term “transformed” cell means a cell that is able to grow and reproduce without restriction given ample nutrients, but that does not exhibit contact inhibition. Transformed cells will form tumors when injected into immunocompromised mice.

The term “heterologous” means derived from a separate genetic source, a separate organism, or a separate species. Thus, a heterologous antigen is an antigen from a first genetic source expressed by a second genetic source. The second genetic source is typically a vector.

The term “antigen” means any substance that elicits an immune response in an organism. The immune response can be cellular, humoral, or a combination thereof. An antigen can have more than one epitope.

The term “epitope” means a particular site of a molecule to which an antibody binds or a particular fragment of a polypeptide that is recognizable by T lymphocytes when presented in a molecular complex with MHC proteins.

The term “recombinant” generally refers to a non-naturally occurring nucleic acid ornucleic acid construct. Such non-naturally occurring nucleic acids include combinations of DNA molecules of different origin that are joined using molecular biology technologies, or natural nucleic acids that have been modified, for example that have deletions, substitutions, inversions, insertions, etc. Recombinant also refers to the polypeptide encoded by the recombinant nucleic acid. Non-naturally occurring nucleic acids or polypeptides include nucleic acids and polypeptides modified by man.

The term “null mutation” means a mutation or change in a gene that results in the gene not being transcribed into RNA and/or translated into a functional protein product. Null mutations include, but are not limited to deletions, insertions, point mutations, transpositions, inversions, and substitutions.

The term “immunomodulator” means a substance that increases or decreases an immune response in a host. Exemplary immunomodulators include, but are not limited to chemokines and cytokines.

2. Recombinant MVA-based Vectors

One embodiment of the disclosure provides a recombinant modified vaccinia Ankara virus (rMVA) that expresses at least one heterologous antigen and does not express a gene required for replication of the rMVA, for example vaccinia uracil DNA glycosylase (udg), also referred to as MVA101R. It will be appreciated that udg can be mutated in the rMVA to prevent the expression of a functional UGD^{MVA} protein, also referred to as a null mutation. The genomic sequence of MVA is known in the art and is available at GENBANK Accession No. U94848 and in Antoine G. et al. “The complete genomic sequence of the modified vaccinia Ankara strain: comparison with other orthopoxviruses”; Virology 244(2):365-396(1998), both of which are incorporated by reference in their entirety. Representative mutations of udg include, but are not limited to, an insertion, inversion, deletion, point mutation, single base substitution, etc. In a particular embodiment, udg is deleted in whole or part from the rMVA.

The heterologous antigen can be any polynucleotide or polypeptide that is capable of eliciting an immune response in a mammal, for example primates including humans. Exemplary heterologous antigens include, but are not limited to, viral polypeptides other than MVA polypeptides, bacterial polypeptides, recombinant polypeptides specific for cancer cells, fungal polypeptides, plant polypeptides, and polypeptides of parasitic organisms. Representative heterologous viral antigens include, but are not limited to, HIV antigens, measles antigens, flavivirus antigens, small pox antigens, hepatitis antigens, adenoviridae antigens, alphavirus antigens, arbovirus antigens, borna disease antigens, bunyaviridae antigens, caliciviridae antigens, chickenpox-antigens, coronaviridae antigens, coxsackievirus antigens, cytomegalovirus antigens, dengue virus antigens, arbovirus antigens, hemorrhagic fever antigens, herpesviridae antigens, influenza antigens, mumps antigens, paramyxoviridae antigens, rabies antigens, respiratory syncytial virus antigens, rubella antigens, West Nile fever antigens, SARS antigens, and Yellow fever virus antigens.

Another embodiment provides a vaccine including a rMVA vector that expresses at least one heterologous antigen and contains at least on null mutation in a vaccinia gene required for replication of the rMVA, for example uracil DNA glycosylase (UDGmvA) and optionally, a pharmaceutically acceptable carrier or excipient.

2.1 Methods of Propagating Modified MVA Vectors

Another embodiment provides a method for the propagation or manufacture of the disclosed modified MVA vectors. MVA has been traditionally propagated on primary chicken embryo fibroblasts (CEFs) (which are inconvenient to derive), and due to its host-range restriction for chicken cells (and limited other cell lines), the cells available to derive and propagate MVA were severely limited. Further, although CEFs can be prepared from specific pathogen free (SPF) animals, chicken genomes commonly harbor numerous endogenous retroviruses that represent potentially undesirable adventitious agents in vaccine preparations. Apart from primary CEFs, the only other cells currently available that can propagate MVA are transformed cell lines. Transformed cell lines are unlikely to be deemed acceptable for the generation of vaccines because of the tumorigenic nature of the cell line.

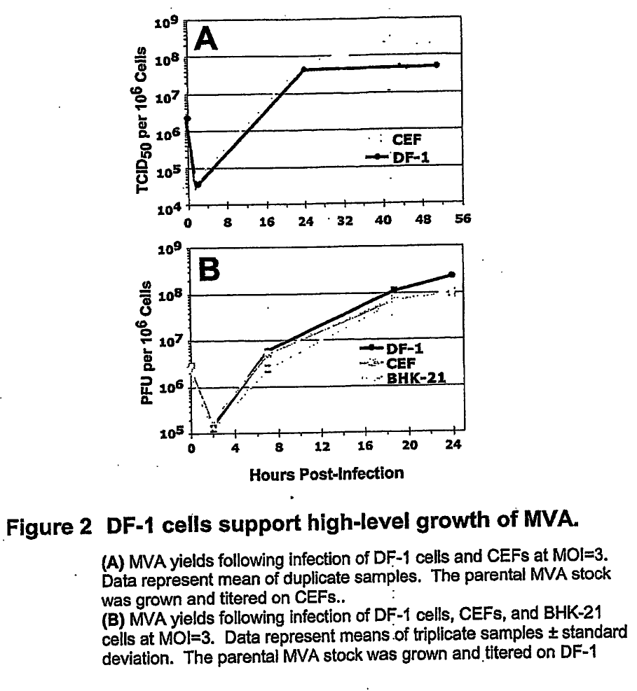

It has been discovered that an immortalized, but not transformed, CEF cell line (termed DF-1), derived from the endogenous retrovirus-free EV-O chickens that are free of endogenous avian sarcoma and leukosis viruses, grows MVA as well as CEFs, but is far more convenient (and potentially safer) than CEFs (FIG. 2). Accordingly, one embodiment of the present disclosure provides a method of propagating MVA in an immortalized, non-transformed cell line, in particular an avian fibroblast cell, more particularly a DF-1 cell. The method includes infecting an immortalized, non-transformed chicken embryo fibroblast with MVA, in particular a recombinant MVA expressing at least one heterologous antigen. The infected cells will form plaques from which the virus can be isolated and optionally purified using conventional techniques.

2.2 Transfer and Expression Vectors

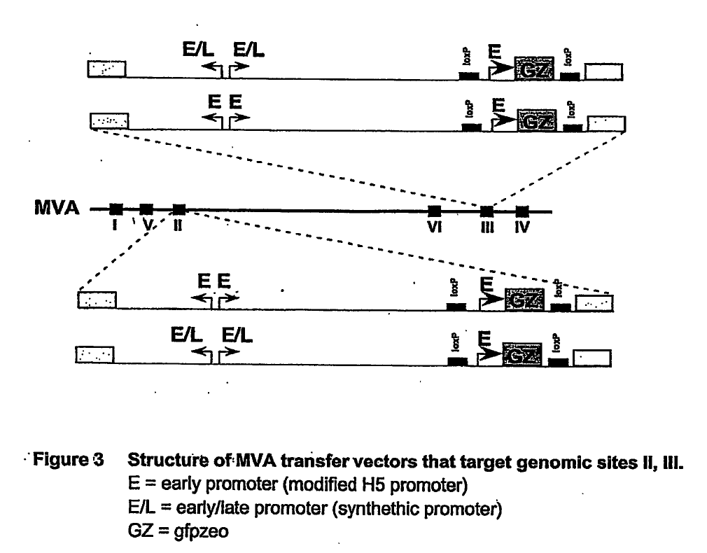

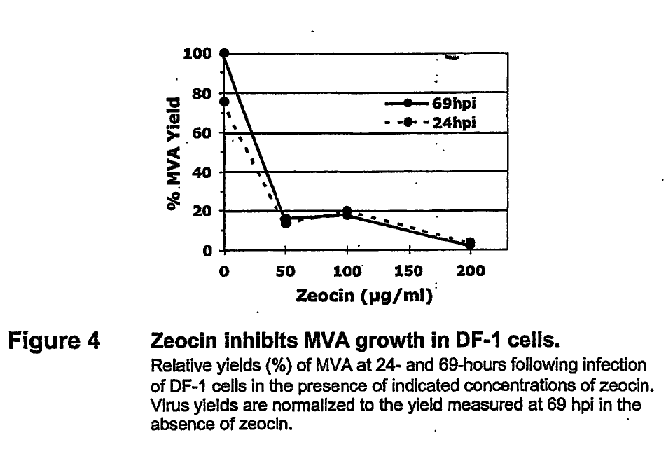

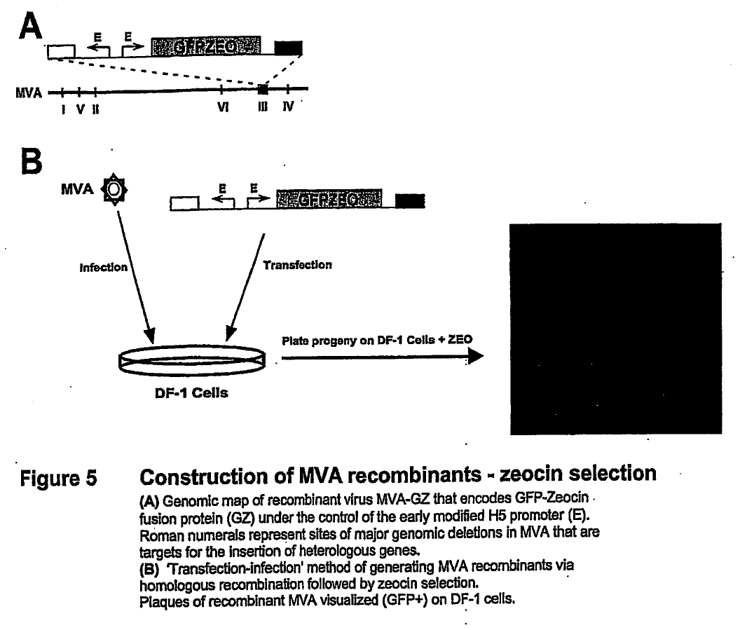

Another embodiment provides transfer and expression vectors that enable facile insertion of heterologous sequences into positions in the MVA genome that flank defined deletions (referred to as sites II and III) that do not code for intact MVA gene products. Certain embodiments provide transfer and expression vectors having “bi-directional” promoters that have been modified to express high levels of a protein at early (E) or both early and late (E/L) times in the W life cycle (FIG. 3). These transfer vectors also encode a dominant selectable marker that has been shown to enable rapid selection for (and isolation of) recombinant MVAs. An exemplary dominant selectable marker is a genetic fusion (gfp-zeo, Invitrogen) of the green fluorescent protein gene (gfp) to a gene encoding resistance to the antibiotic zeocin [zeo]). Because MVA replication in DF-1 cells is blocked in the presence of zeocin (200 μg/ml, see FIG. 4), viral recombinants that have incorporated gfpzeo into their genome may be rapidly selected for in the presence of zeocin and rapidly identified/isolated through the microscopic detection of GFP+ plaques (FIG. 5). These vectors permit for the generation of recombinant MVAs via the standard “transfection-infection’-homologous recombination protocol and the simultaneous insertion and high-level expression of up to four exogenous genes. High-level expression means higher expression than levels of existing typical eukaryotic plasmid expression vectors.

In addition, capacity to express foreign genes from MVA has been expanded by incorporating additional early promoter elements into the site II, III transfer vectors and by creating analogous transfer vectors (each with 2 early promoters) that target insertion to genomic loci other than sites II or III. These targeted loci encode essential viral gene products and/or immune evasion genes and include the following coding regions: udg, D7R, IL-1βr, A46R, IL18BP, A41L, and E3L (see below). Other embodiments provide recombinant MVAs that express high levels of heterologous viral antigens, including but not limited to HIV and LCMV antigens, or cellular gene products including but not limited to GM-CSF or MIP3alpha.

An exemplary transfer vector includes an expression cassette comprising a dominant resistance marker operably linked to at least two promoters, wherein the expression cassette is flanked by sequences complementary to at least a portion of udg, D7R, IL-1βr, A46R, IL18BP, A41L, or E3L. The flanking sequences are typically 500 bp on each side of the expression cassette. The promoters are generally promoters that regulate the expression of early genes in the lifecycle of MVA, and include, but are not limited to the mH5 promoter.

The disclosed transfer vectors can be used to generated rMVA and vectors thereof. Generally, the expression cassette recombines into the MVA genome. In one embodiment, the recombination is achieved via homologous recombination in permissive cells, for example DF-1 cells, that have been infected with MVA or rMVA and simultaneously transfected with the plasmid transfer vector. Resulting recombinant MVA viruses may be selected/screened for using the dominant selection marker, for example antibiotic resistance.

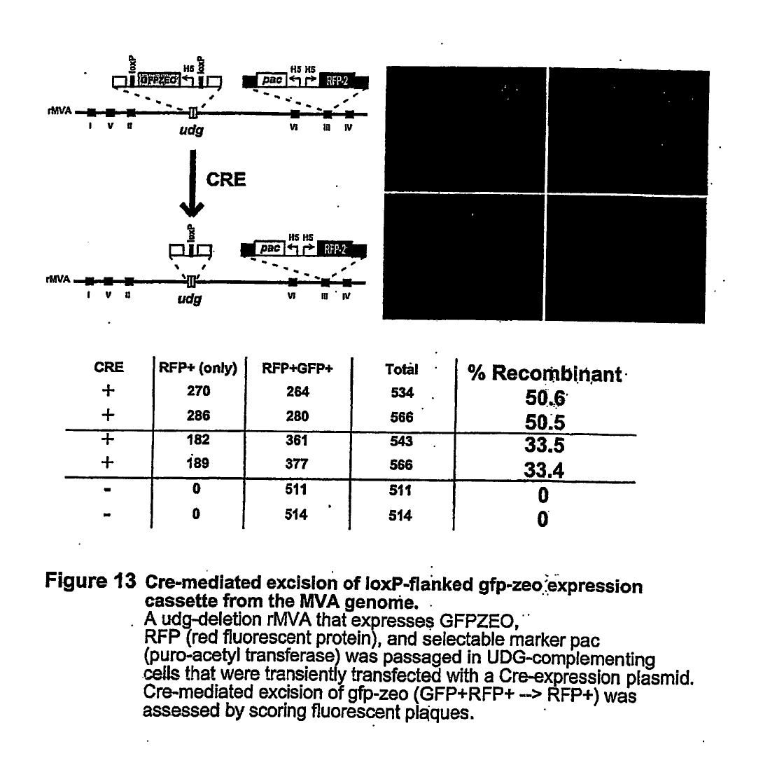

In another embodiment the expression cassette comprises a target nucleic acid sequence flanked by loxP sites. The DF-1 cell can be engineered to express a site-specific recombinases such as Gre (or Flp). The engineered DF-1 cell can be transfected with the vector containing the expression cassette and infected with MVA. The expression cassette can then be inserted into the MVA via homologous recombination. Cre recombinase specifically recognizes short (˜30 bp) DNA elements (loxP sites) and mediates genetic recombination between two loxP sites. Recombination can be directed to result in either insertion of DNA into a target sequence at a loxP site or deletion of DNA from between loxP sites within the target sequence. Both transient and constitutive expression of Cre recombinase in DF-1 cells mediates efficient excision of DNA sequences from the MVA genome that are flanked by loxP sites (FIG. 13).

Alternatively, a DF-1 cell engineered to express Cre recombination can be infected with a rMVA comprising two loxP sites flanking a region of interest in the rMVA. By controlling the expression of Cre recombinase in the DF-1 cell and using a transfer vector, predetermined heterologous nucleic acid sequences can be inserted into the rMVA between the loxP sites. The heterologous nucleic acid sequence can encode an antigen, chemokine, cytokine, anti-apoptotic factor, or combinations thereof.

In another embodiment, site-specific recombination may be achieved through trans-membrane delivery of a modified cell-permeable Cre recombinase protein that is engineered to be targeted to the cytoplasm, rather than the nucleus. Following cre-lox excision of a gfp-zeo expression cassette from a MVA recombinant, additional genes can be sequentially added at other insertion sites in the MVA genome using the same gfp-zeo selection process (and at the original site via cre-mediated insertion). In addition, when deletion of a specific gene is desired, a transfer vector can be created that replaces the gene of interest with the loxP flanked mH5-promoter gfp-zeo cassette (which is itself flanked by MVA sequences that surround the gene), and used as described above to generate recombinant viruses lacking specific genes.

The use of site-specific recombination to genetically engineer MVA (and likely, other poxviruses) is an improvement over current technology because the frequency of site-specific recombination is much greater than that attainable via homologous recombination. The use of cre/loxP recombination to modify the MVA genome is expandable to encompass the use of alternative site-specific recombinase systems (e.g., Flp recombinase/frt) either singly, or in combination with cre/loxP.

2.3 Genetic Systems for the Generation of MVA-deletion Vaccine Vectors

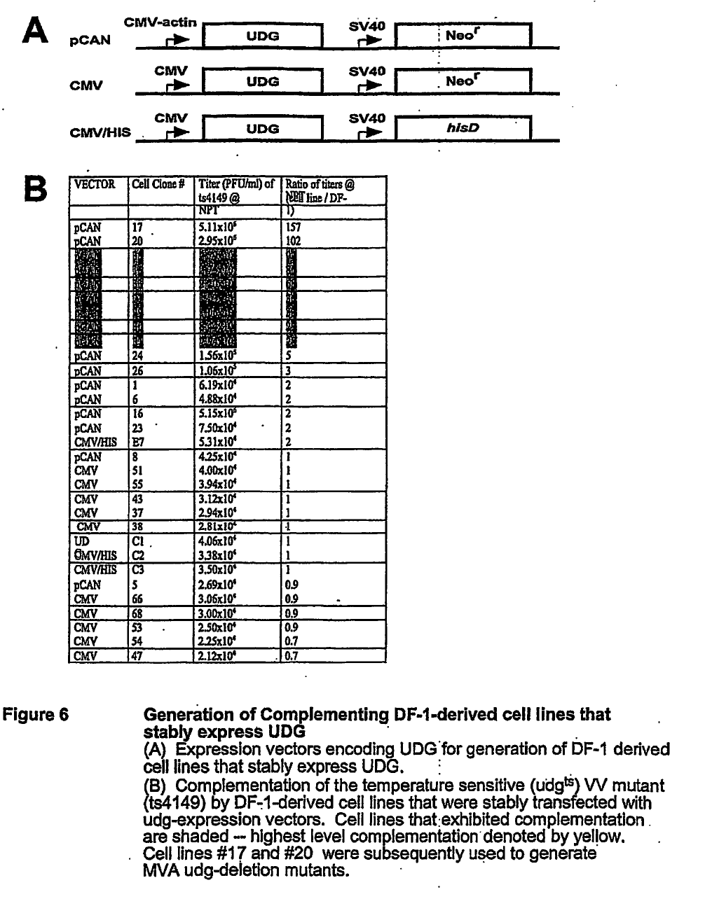

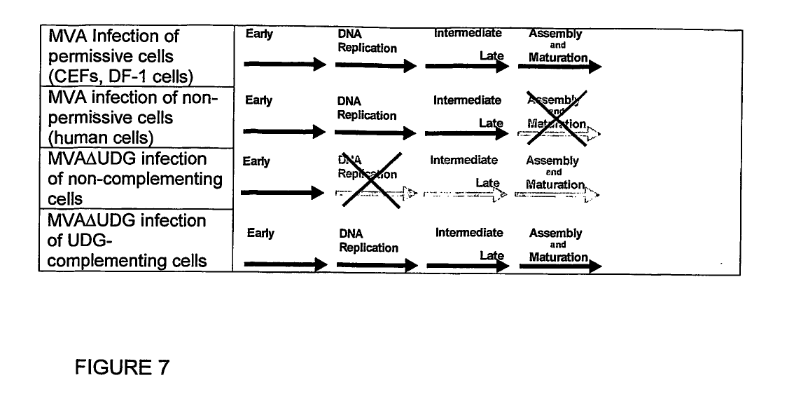

Still another embodiment provides a system for the generation of MVA-deletion mutants. MVA-deletion mutants typically have a deletion in one or more essential MVA viral genes. Although other vaccinia virus vectors have been reported which have deletions in essential viral genes, no MVA-deletion mutant vectors are described in the art because MVA-generally requires its propagation on primary chicken embryo fibroblasts. As described above, the present disclosure provides methods and compositions for propagating MVA in an immortalized (but not transformed) chicken fibroblast cell line (DF-1). These immortalized cells can be stably transfected to express MVA genes, including essential viral genes (FIG. 6). This allows, for the first time, generation and propagation of mutant MVAs that harbor deletions of (otherwise) essential genes from the viral genome through functional complementation in trans (FIG. 7).

One embodiment provides a system for propagating recombinant MVA including a recombinant MVA having a deletion in an essential MVA gene, and an immortalized, but not transformed, avian fibroblast cell transfected to express the deleted MVA gene. The complementing DF-1-derived cell lines can constitutively expresses the deleted MVA gene and thereby supply the deleted MVA gene product so that the rMVA is able to propagate within the transfected cell.

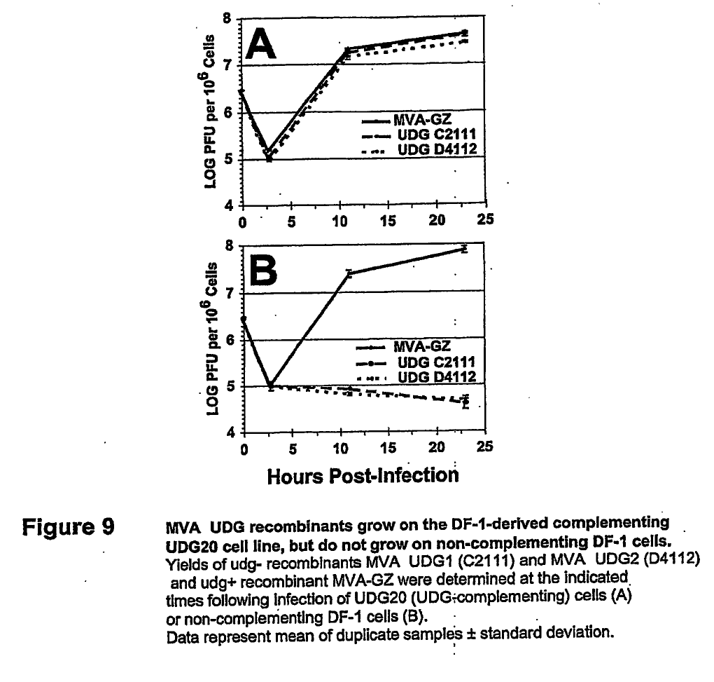

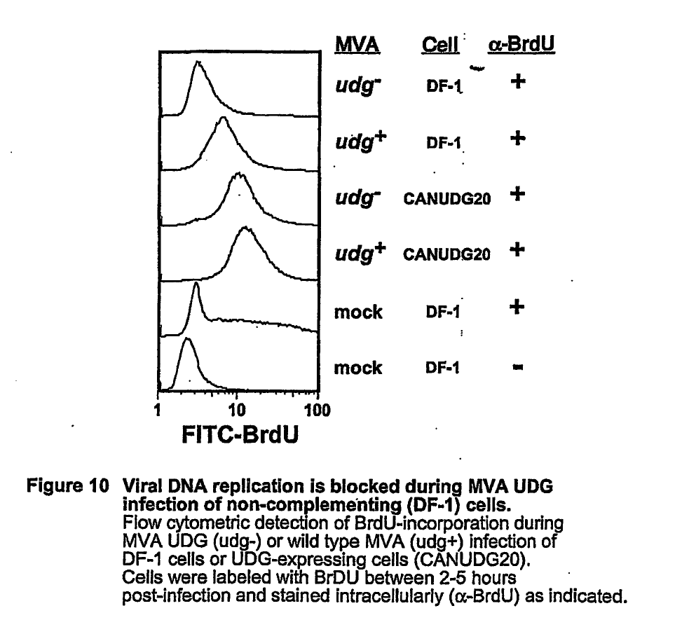

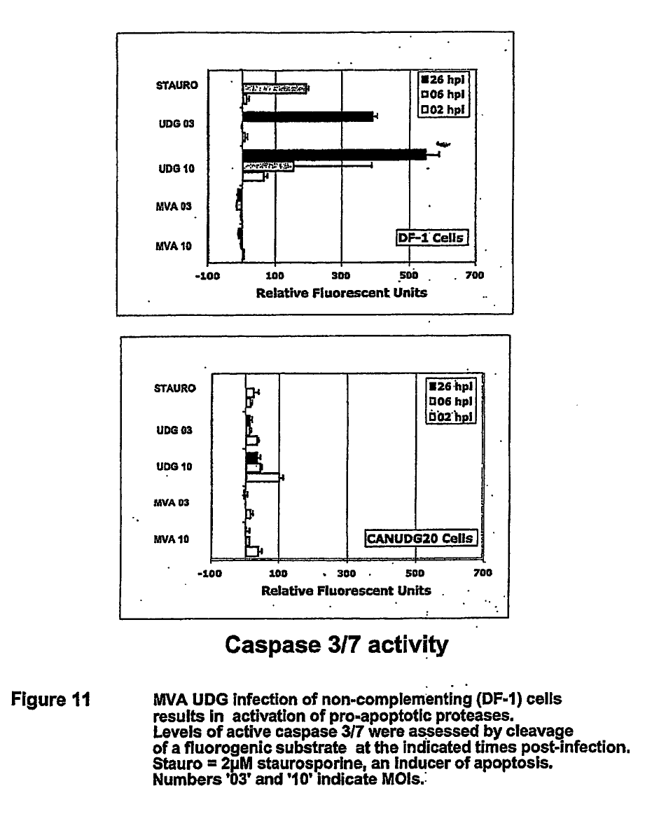

In another embodiment, the nucleic acid construct expressing the deleted MVA gene is incorporated into the genome of the cell. An essential MVA gene means a gene that is required for the propagation or survival of MVA, for example a gene that encodes a protein necessary for viral replication. Representative essential MVA genes include, but are not limited to, udg, A28L and D7R. As a representative example, the udg-deletion mutant has been shown to require the complementing (udg-expressing) DF-1-derived cell line for permissive replication (FIG. 9). Moreover, the replication cycle of this mutant is blocked (during infection of non-complementing cells) at the level of DNA replication (FIG. 10) and infection of non-permissive cells results in apoptosis (FIG. 11).

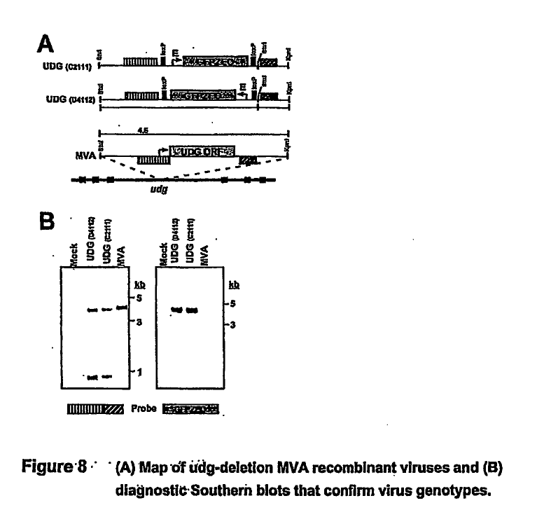

Another embodiment provides, MVA vectors that elicit augmented immune responses against encoded heterologous antigens, for example HIV antigens, compared to existing MVA vectors while minimizing undesirable responses against the vaccine vector itself. An exemplary MVA vector includes one having a udg-deletion (FIG. 8) causing the virus to be genetically blocked midway during the VV replication cycle and express only a subset of MVA genes (FIG. 7). Immunization with this deletion vector (or with vectors derived from other mutants that are also blocked midway through the VV replication cycle) can help focus the host immune response against the relevant heterologous antigens encoded by the vaccine vector through reduction of the number of irrelevant, endogenonous MVA antigen targets that are expressed. Moreover, mutants blocked for expression of late MVA genes, including those that encode highly immunogenic MVA structural proteins, elicit significantly lower neutralizing immune responses against the MVA vector itself—thereby, enabling subsequent immunizations, with the same vector, to effectively boost immune responses against encoded HIV antigens. Finally, premature abortion of the VV replication cycle that occurs during infection with these deletion mutants results in increased apoptosis of infected cells and concomitant enhancement of cross-presentation of vector-encoded antigens to DCs, thereby increasing the overall duration and magnitude of desired immune responses.

Yet another embodiment provides a recombinant modified vaccinia Ankara virus modified to express a human-codon-optimized HIV-1 consensus sequence polypeptide but is unable to express a functional MVA uracil DNA glycosylation polypeptide. The HIV consensus sequence can encode HIV-1 Gag, Pol, Env, and Nef from Clades B, C, and A, a fragment thereof, or a fusion thereof.

2.4 Modulation of Apoptosis to Enhance Immunogenicity of MVA-Based Vaccine Vectors

Although it has been commonly assumed that vaccinia virus (VV) has broad cellular tropism, it has been discovered that both W and the attenuated strain MVA preferentially target dendritic cells (DCs) for infection in vivo. Because DCs are essential antigen presenting cells for generation of immune responses, this unexpected in vivo tropism is a favorable attribute for potential vaccine vectors. Both W and MVA rapidly induce apoptosis of infected DCs, coincident with the transition from “early” to “late” VV gene expression programs. The rapid killing of DCs by VV and MVA likely limits the capacity of infected DCs to elicit maximally potent immune responses. As such, other embodiments of the disclosure provide methods that prolong the survival of infected DCs by delaying or blocking the transition to the later stages of the vaccinia life cycle to enable increases in the immunogenicity of the disclosed vaccinia-based vaccine vectors.

The availability of MVA and W recombinants that express GFP made it possible to define, using multi-parameter flow cytometry, the target cell tropism for virus infection, as well as the fate of infected cells. Although VV infects a broad range of cells in culture, it was discovered that VV tropism is highly restricted among primary hematolymphoid cells in both mice and humans. In studies of VV and MVA infection of human peripheral blood cells (PBMCs), macrophages were found to be readily infectable by VV and MVA, with B cells somewhat less susceptible and resting T cells resistant. As dendritic cells (DCs) are essential antigen presenting cells (APCs) necessary for generation of immune responses, but are quite rare in the PBMC population, the susceptibility to infection of murine and human DCs derived ex vivo according to standard methods were examined.

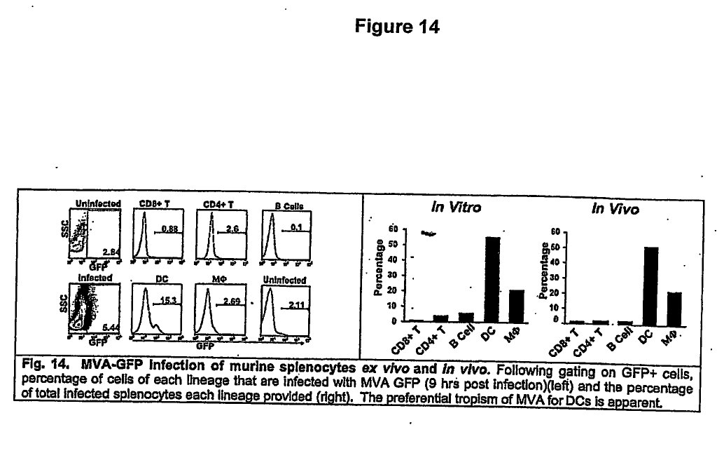

Both mature and immature murine bone marrow-derived DCs (BMDCs) and human monocyte-derived DCs (MMDCs) were found to be highly susceptible to VV and MVA infection ex vivo (see FIG. 14 for murine data).. Furthermore, it was found that priiary DCs freshly isolated from mouse spleens, as well as both human myeloid (CD11c+) and plasmacytoid (CD123+) DCs freshly isolated from peripheral blood, are readily susceptible to VV and MVA infection ex vivo. When murine splenocytes are infected with either VV-GFP or MVA-GFP ex vivo, DCs were found to be most highly susceptible cell type for infection, followed by macrophages and B cells. However, when virus infections were carried out in vivo (by IV injection), the degree of preferential infection of DCs by MVA and VV was even more pronounced (FIG. 14). In all, these data indicate that VV and MVA are selective in their target cell preference, with a predilection for infection of DCs, the immune system's most important antigen presenting cells.

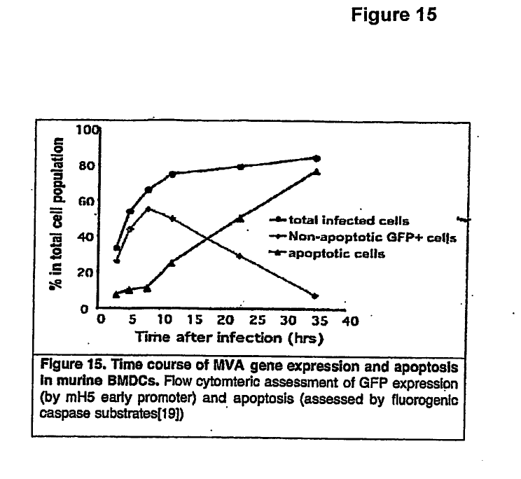

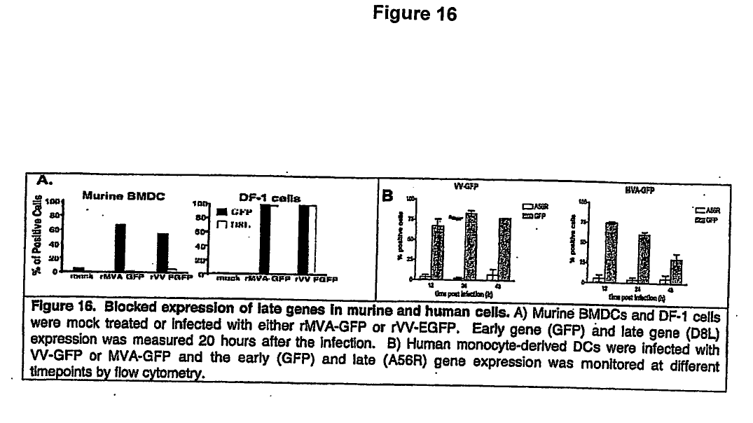

How virus-DC interaction affects generation of immune responses to VV and MVA-based vectors (or VV and MVA themselves) was also investigated. The availability of viruses encoding GFP enabled the fate of infected cells to be tracked, both with respect to infected cell longevity and the VV life cycle within specific cell types. Two major findings that are of direct relevance to the derivation of more effective vaccine vectors were discovered. First, while VV and MVA have the desirable property of infecting DCs, they also induce the prompt apoptosis of both immature and mature DCs. Indeed, MVA induces DC apoptosis earlier following infection than otherW strains (e.g., NYCBH, WR), perhaps because MVA lacks the anti-apoptotic VV SPI-2 gene (see FIG. 15). Second, progression through the viral replication cycle is abortive following infection of DCs with VV or MVA, with readily detectable early gene expression, but little if any late gene expression (see FIG. 16). This phenomenon of virus induced DC apoptosis and abortive virus infection has been observed in both in vivo isolated and tissue culture derived DC populations in both humans and mice. Collectively, these data help explain earlier observations that expression of heterologous genes from early VV promoters results in induction of higher level protective immunity to both infectious agents and model tumors than from the typically stronger late promoters. Further, these observations help explain why MVA is a very effective vector in boosting immune responses to antigens primed previously by heterologous vectors (especially when early VV promoters are used).

Accordingly, one embodiment provides modified viral vectors that express at least one heterologous antigen as well as gene products that inhibit or promote apoptosis of infected cells. An exemplary modified vector expresses at least one anti-apoptosis factor or pro-apoptotic factor of viral origin, cellular origin, or both, in combination with at least one heterologous antigen, for example a viral antigen including, but not limited to an HIV antigen or LCMV antigen. The heterologous DNA encoding the heterologous polypeptides can be placed in the region of deletion sites II, III, or both in the MVA genome. Representative anti-apoptosis factors that can be expressed by the disclosed modified vectors, includes, but is not limited to: Bcl-xl (SEQ ID NO.1-2), Bcl2 (SEQ ID NO 34), Ciapl, Ciap2, Flame (SEQ ID NO.5-6 ), CrmA (SEQ ID NO. 7-8), p35, Xiap (SEQ ID NO.9-10), MC159. Representative pro-apoptoticfactors include, but are not limited to Bax, Bak, Bid, Fas receptor, AIF, and caspase 3-CPP32.

2.5 MVA-based Vaccine Vectors Encoding Immunomodulators

Another embodiment provides modified MVA vectors that facilitate the recruitment of DCs to the site of immunization, promotion of DC maturation, and modulate DC lifespan following MVA infection. Exemplary modified vectors express one or more immunomodulators, including but not limited to specific cytokines, chemokines, or combinations thereof. The presentation of MVA-encoded antigens occurs by both direct and indirect pathways. Embodiments of the disclosed modified vectors express immunomodulators that promote enhanced antigen presentation by either the direct or indirect pathways, or both. Exemplary immunomodulatory gene products expressed in recombinant MVAs include, but are not limited to MIP3α (SEQ ID NO. 11-12), GM-CSF (SEQ ID NO. 13-14), IL-15 (SEQ ID NO. 15-16), or combinations thereof.

GM-CSF is a cytokine with pleiotropic beneficial activities in augmenting immune responses, including promotion of DC expansion and maturation, and has been explored with promising results in a number of infectious disease and tumor vaccine models. IL-15 is a cytokine that resembles IL-2 biologically, stimulating NK cells, T cells and B cells to become activated and proliferate. IL-15 also promotes dendritic cell activation and protects them from apoptosis, as well acting to regulate memory T cell homeostasis. IL-15 serves as an adjuvant in DC-based vaccine studies in mice, and incorporation of IL-15 into VV attenuates viral pathogenicity in murine infection models. The CC chemokine macrophage inflammatory protein (MIP)-3α (CCL20) plays a key role in recruitment of immature DCs to sites of inflammation and infection (in particular, in trafficking of Langerhans cells to the skin). Recently, MIP-3α expressed by a recombinant adenovirus has been reported to promote tumor clearance in a tumor vaccine model.

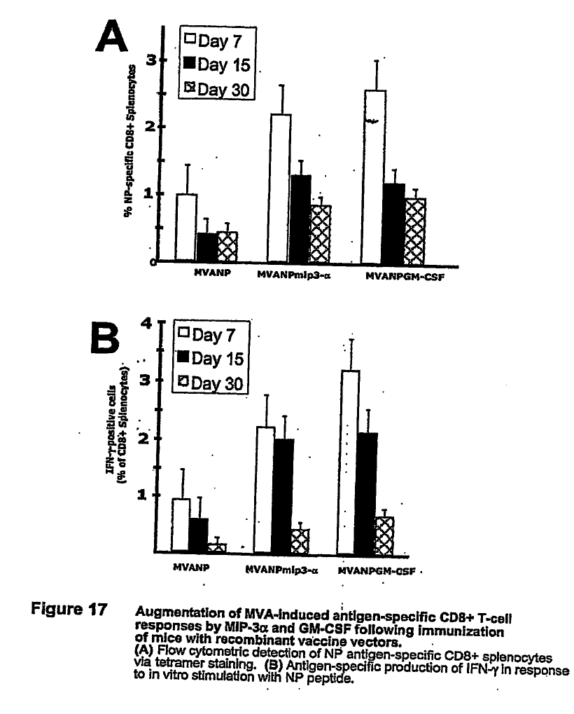

In some embodiments, expression of GM-CSF, IL-15, and MIP3alpha from the genomes of rMVA vectors each resulted in two to four-fold enhancement of cellular immune responses to heterologous antigens (e.g., LCMV NP) that are also expressed from the MVA vector (FIG. 17). Given that these immunomodulators have different targets and mechanisms of action, another embodiment provides a modified MVA vector expressing a heterologous antigen and a plurality of immunomodulators.

One exemplary MVAII-hMIP3α/hGM-CSF transfer vector has a sequence of (SEQ ID NO. 17). Particular features of the transfer vector are provided in Table 2.

TABLE 2
Annotation of MVAII-hMIP3α/hGM-CSF
transfer vector sequence:

| Nucleotide range | Feature |
|---|---|
| 6959-7173, 0-352 | MVA II Flank 1 |
| 579-546 | loxP site |
| 1684-586 | gfpzeo |
| 1784-1715 | pH5 |
| 1825-1792 | loxP site |
| 2710-2276 | hGM-CSF |
| 2950-2881 | pH5 |
| 2956-3026 | pH5 |
| 3141-3431 | hMIP3α |
| 3585-4248 | MVA II Flank 2 |

2.6 MVA Vectors with Deletions of the Immune Evasion Genes

Although MVA lacks a number of viral immune evasion genes including the soluble receptor for IFNγ, IFNα/β, TNF, and CC-chemokines, a number of known and candidate immune evasion genes remain intact. One embodiment provides a modified MVA vector in which at least one of the following is deleted, not expressed, or non-functional: IL1β Receptor, A46R, IL-18BP, A41L, E3L, or a combination thereof. The virus or vector with the null mutation, for example a deletion, can also express at least one cytokine or chemokine and at least one heterologous antigen.

MVA encodes an intact IL-1 receptor gene (IL-1βR: gene B15R in WR) whose product is secreted from virus-infected cells and binds IL-1β with high affinity. As IL-1β is a cytokine produced in response to infection and bssue injury, and exerts multifaceted effects in regulation of inflammatory and immune responses, deletion of the IL-1βR gene from the WR VV strain or repair of the defective gene in VV Copenhagen substantially alters viral pathogenesis in vivo. Given the importance of the IL-1 signaling in adaptive immunity, and in linking innate and adaptive immune responses, deletion of the MVA IL-1βR can provide enhanced anti-VV responses.

VV A46R (159R in MVA) encodes a protein with sequence homology to the Toll//IL-1 receptor (TIR) domain motif that defines the IL-1/Toll-like receptor (TLR) superfamily of receptors that play key roles in innate immune responses and in inflammation. The VV A46R gene product is reported to inhibit IL-1-mediated activation of NF-KB: a key step in activation of host innate and adaptive responses to infection. Deletion of MVA 159R can result in augmentation of MVA immunogenicity (for example, by acting synergistically in a MVA genetic background where IL-1βR is also absent).

Most orthopoxviruses (including W and MVA:[C12L]) encode a secreted protein that binds IL-18, effectively inhibiting activation of IFN-γ production from T, B and NK cells. Acting in synergy with IL-12, IL-18 plays a very important role in marshalling Th1 cellular immune responses, The deletion of IL-18BP from VV does not affect virus replication in culture, but attenuates viral pathogenic potential in vivo (by as yet incompletely understood mechanisms). Deletion of the intact IL-18BP from MVA can result in induction of enhanced antiviral immune responses.

The VV (and MVA)-encoded A41 L gene product shares amino acid sequence homology with the vCKBP protein (a CC-chemokine binding protein encoded by some VVs) and the GIF protein (that binds GM-CSF and IL-2) encoded by the orf virus. The A41L protein is known to be secreted from infected cells; however, its ligand has not yet been identified. Deletion of the A41L gene from VV does not impair replication in culture, but results in increased inflammatory infiltration and accelerated virus clearance following intradermal inoculation of mice. A41L likely encodes an immunomodulator whose deletion from MVA can promote generation of antiviral immune responses.

MVA (and other VVs) encode E3L homologs that act to sequester dsRNA and prevent PKR-mediated induction of type-I interferons that induce anti-viral state and inhibit viral gene expression and replication. Deletion of E3L from VV affects viral host range and augments apoptosis to enhance cross-presentation of vector-encoded antigens.

2.7 MVA Vectors Having Consensus Antigens

Rapid virus evolution in vivo is a fundamental aspect of the biology of HIV infection that contributes significantly to the elusiveness of this pathogen from host antiviral responses and remains a formidable challenge to the development of effective vaccines against HIV/AIDS. The capacity of HIV to evolve rapidly in vivo to escape recognition by host cellular and humoral immune responses is due to the high error rate of reverse transcriptase acting in concert with a high level of virus replication/turnover that is typically seen during untreated HIV infection. It has been estimated that, during chronic infection, HIV variants harboring single nucleotide mutations at every position of the viral genome have the potential of arising thousands of times per day. In a relatively short time, such enormous capacity for generating diversity results in a vast pool of mutants from which host immune responses may select for resistant variants. Thus, to curb the generation of (and establishment of latent infection by) potential immune escape variants, vaccine-elicited CD8+ T-cell responses will likely need to control HIV replication at the very earliest stages of infection.

In order to achieve early immune recognition and containment of HIV, vaccine-elicited CD8+ T-cell responses should target, a maximal number of epitopes that are identical (or nearly so) to those in the strain of HIV most likely to be subsequently encountered. However, choosing which antigens to include in candidate vaccines is further complicated by the enormous worldwide diversity of HIV variants. Phylogenetically diverse HIV subtypes are composed of HIV variants whose gene products may diverge by 15% (Gag) to 30% (Env) at the amino acid level. In contrast, amino acid changes of less than 2% between a vaccine strain and circulating forms of influenza virus necessitate a change in vaccine strains. High levels of genetic recombination further increase the diversity of HIV by generating viruses with inter-subtype mosaic genomes (circulating recombinant forms, CRFs).

Because phylogenetically diverse HIV subtypes are prevalent in different geographical locations, the use of HIV subtype-consensus sequences as vaccine immunogens has been proposed as a scientifically justifiable and feasible approach towards trying to diminish the problem that diversity poses against selecting relevant antigens for inclusion in candidate HIV vaccines.

One embodiment provides a modified MVA vector that expresses a clade-specific HIV-1 consensus gene product (4 consensus genes×3 HIV clades=12 total genes). Another embodiment provides HIV vaccine compositions. For use as candidate HIV/AIDS vaccines, human-codon-optimized consensus sequences encoding HIV-1 Gag, Pol, Env, and Nef (from HIV Clades B, C, A) have been synthesized, subcloned into MVA-expression vectors, and recombined into both wild-type and deletion-mutant MVA vectors.

Exemplary optimized consensus sequences include, but are not limited to: HIV subtype A gag gene (SEQ ID NO.18), HIV subtype A Gag protein (SEQ ID NO. 19), HIV subtype A pol gene (SEQ ID NO. 20), HIV subtype A Pol protein (SEQ ID NO. 21), HIV subtype A env gene (SEQ ID NO. 22), HIV subtype A Env protein (SEQ ID NO. 23), HIV subtype A nef gene (SEQ ID NO. 24), HIV subtype A Nef protein (SEQ ID NO. 25), HIV subtype B gag gene (SEQ ID NO. 26), HIV subtype B Gag protein (SEQ ID NO. 27), HIV subtype B pol gene (SEQ ID NO. 28), HIV subtype B Pol protein (SEQ ID NO. 29), HIV subtype B env gene (SEQ ID NO. 30), HIV subtype B Env protein (SEQ ID NO. 31), HIV subtype B nef gene (SEQ ID NO. 32), HIV subtype B Nef protein (SEQ ID NO. 33), HIV subtype C gag gene (SEQ ID NO. 34), HIV subtype C Gag protein (SEQ ID NO. 35), HIV subtype C pol gene (SEQ ID NO. 36), HIV subtype C Pol protein (SEQ ID NO. 37), HIV subtype C env gene (SEQ ID NO. 38), HIV subtype C Env protein (SEQ ID NO. 39), HIV subtype C nef gene (SEQ ID NO. 40), and HIV subtype C Nef protein (SEQ ID NO.41).

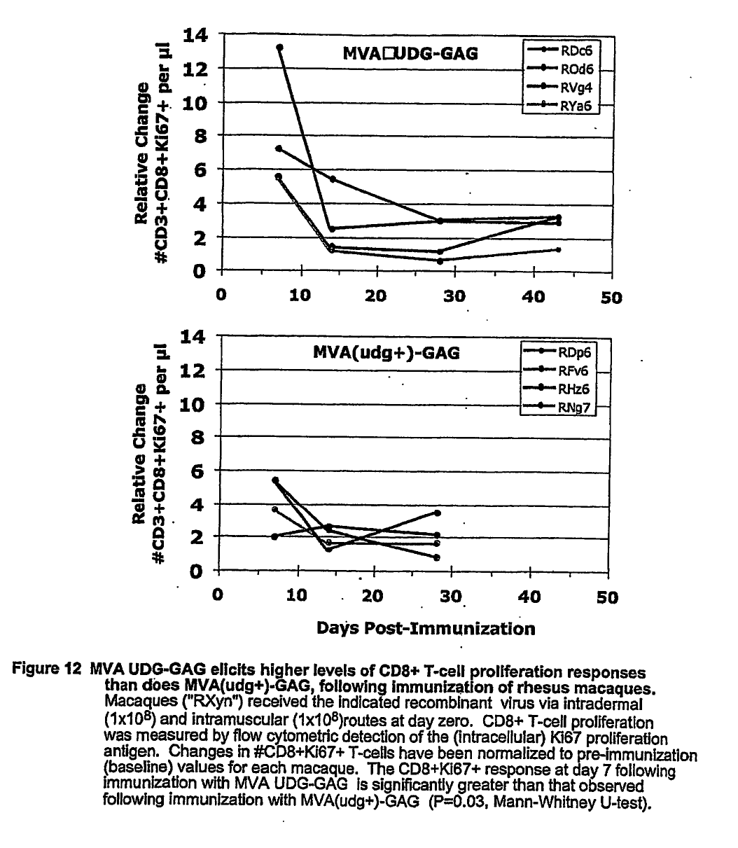

Exemplary fusion genes and proteins include, but are not limited to: HIV subtype A gag-polfusion gene (SEQ ID NO.42), HIV subtype A Gag-Pol fusion protein (SEQ ID NO. 43), HIV subtype A env-nef gene (SEQ ID NO.44), HIV subtype A Env-Nef fusion protein (SEQ ID NO. 45), HIV subtype A gag-pol-nef gene (SEQ ID NO. 46), HIV subtype A Gag-Pol-Nef fusion protein (SEQ ID NO. 47), HIV subtype A gag-pol-env-nef gene (SEQ ID NO. 48), HIV subtype A Gag-Pol-Env-Nef protein (SEQ ID NO. 49), HIV subtype B gag-pol fusion gene (SEQ ID NO. 50), HIV subtype B Gag-Pol fusion protein (SEQ ID NO. 51), HIV subtype B env-nef fusion gene (SEQ ID NO. 52), HIV subtype B Env-Nef fusion gene (SEQ ID NO. 53), HIV subtype B gag-pol-nef fusion gene (SEQ ID NO. 54), HIV subtype B Gag-Pol-Nef fusion protein (SEQ ID NO. 55), HIV subtype B gag-pol-env-nef fusion gene (SEQ ID NO. 56), subtype B Gag-Pol-Env-Nef fusion protein (SEQ ID NO. 57), HIV subtype C gag-pol fusion gene (SEQ ID NO.58), HIV subtype C Gag-Pol fusion protein (SEQ ID NO. 59), HIV subtype C env-nef fusion gene (SEQ ID NO. 60), HIV subtype C Env-Nef fusion protein (SEQ ID NO. 61), HIV subtype C gag-pol-nef fusion gene (SEQ ID NO. 62), HIV subtype C Gag-Pol-Nef fusion protein (SEQ ID NO. 63), HIV subtype C gag-pol-env-nef fusion gene (SEQ ID NO. 64), and HIV subtype C Gag-Pol-Env-Nef fusion protein (SEQ ID NO. 65). Immunization of rhesus macaques with MVAΔUDG-GAG (that encodes HIV-1 dade B consensus Gag) results in significantly greater CD8+ T-cell proliferation responses than does similar immunization with MVA(udg⁺)-GAG (FIG. 12).

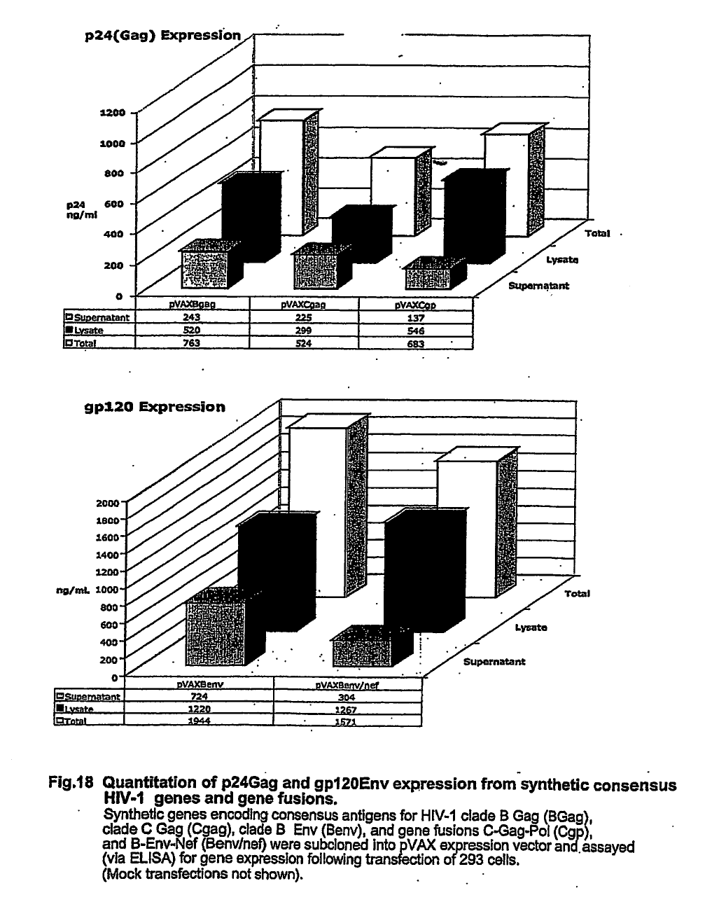

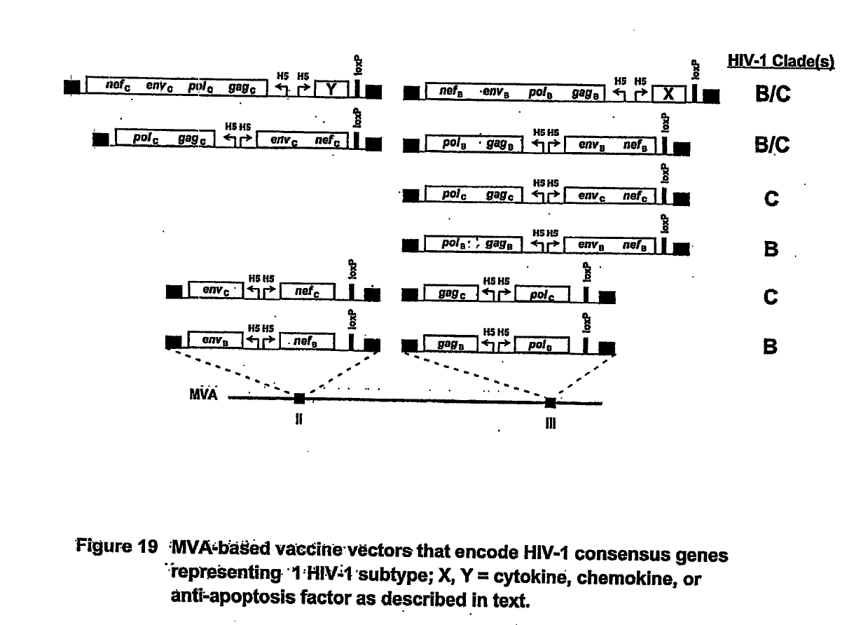

In another embodiment, the nucleotide sequences of all consensus genes have been modified to reflect codon preferences that ensure their maximal expression and translation in human cells. See Table 1 below. In addition, codon-optimization of these HIV genes can optionally result in the removal of inhibitory sequences such that the synthetic HIV-1 genes may be efficiently expressed in the absence of Rev (FIG. 18). Several modifications have also been designed into these synthetic genes to increase their immunogenicity and/or safety profiles for use in human immunizations and include the following: mutation of Asp153 within the catalytic center of reverse transcriptase (RT) to eliminate potential RT activity; deletion of glycine residues (AGly) or mutation of residues (G A) at positions 2 and 3 of Nef to prevent myristoylation and membrane localization of Nef (that is otherwise associated with downregulation of MHC-I molecules); deletion of the first variable loop of Env (ΔV1) or inclusion of V1 from a primary HIV-1 isolate to promote the generation of antibody responses against Env. Finally, signals (5′-TTTTTNT-3′)(SEQ ID NO. 66) that indicate premature termination of transcription from early VV promoters have been removed to allow efficient, full-length expression of HIV genes from early MVA promoters within the viral genome. These consensus HIV genes have been expressed singly (1 promoter: 1gene) or in combination (1 promoter: >1 gene) as gene fusions (gag-pol; gag-pol-nef; env-nef; gag-pol-env-nef) from recombinant MVA vectors. Env-nef fusions have been joined by the FMDV2A proteolytic/slippage sequence to promote independent expression of Env and Nef polypeptides (to prevent Nef insertion into the plasma membrane) and to destabilize the Nef polypeptide (by virtue of N-terminal expression of a proline residue) to promote antigen processing. The use of gene fusions (as opposed to individual genes) effectively increases the expression capacity of the MVA transfer vectors. The expression of HIV-1 consensus genes singly, multiply, and in combination with cytokines, chemokines, and anti-apoptosis factors from MVA-based vaccine vectors is depicted in FIG. 19.

TABLE 1
Salient features of HIV consensus antigens to
be expressed from rMVA-based HIV vaccines.

| | | | MODIFICATIONS* |
|---|---|---|---|
| HIV | | CONSENSUS | [Safety/ |
| SUBTYPES | NOTES | GENES | Immunogenicity] |
| A, B, C | — | gag | — |
| | Independent expression of | pol | Add translation |
| | pol that is not dependent on | | initiation codon |
| | gag-pol frameshift | | |
| | Mutation at RT catalytic | | D153N |
| | center to knock out potential | | |
| | RT activity | | |
| | Augment antibody response | env | V1/V2 (or inclusion |
| | to env | | of YU2 V1/V2) |
| | Prevent myristoylation, | nef | Gly @ aa 2.3 |
| | membrane localization of Nef, | | (or G2A + G3A) |
| | and down-regulation of MHC-I | | |

*All synthetic HIV genes have been codon-optimized for maximum (rev-independent) expression in human cells and exclude early vaccinia transcription termination signals (5′-TTTTTNT-3′) to ensure full length expression from early MVA promoters.

2.8 MVA-based Smallpox Vaccine

The development of the smallpox vaccine-from Jenner to the eradication of smallpox is an especially informative and successful chapter in the history of vaccinology. However, recent concerns about the potential use of smallpox or other highly virulent orthopoxviruses as agents of bioterrorism have led to re-initiation of smallpox vaccination efforts. Vaccination is currently proposed to be initially provided to members of the military, and certain healthcare and emergency service workers, but may also be made widely available to the general public in the future. However, with endemic smallpox now gone, and the prevalence of medical conditions that represent contraindications for VV immunization (e.g., HIV infection and recipients of therapeutic immunosuppression) now markedly increased, the risk-benefit ratio of vaccination is much less favorable than when smallpox was still an endemic disease. Further, even healthy individuals receiving VV immunization have the potential to transmit the vaccine virus to close contacts who may themselves be at risk for serious consequences of VV infection. Importantly, no protective options are now available for individuals who are at high risk of adverse consequences of vaccinia immunization should cases of variola or other pathogenic poxvirus antigens be seen in the future. As such, there is a significant and pressing need to develop much safer and substantially more tolerable vaccines to protect against smallpox and other virulent orthopoxvirus antigens.

The serious and potentially life-threatening complications of VV inoculation were appreciated throughout the smallpox eradication effort. As a result, significant attention was focused on the development of less reactogenic and safer vaccinia immunization strategies—especially those that might be safe in persons at high risk of vaccine-associated adverse events. As inactivated VV vaccines were shown to be ineffective, a variety of more highly attenuated live vaccines were developed as alternatives to the highly effective, yet risky, vaccine preparations (such as Dryvax). However, the lesser reactogenicity of many of the alternative vaccine candidates was matched by unsatisfactory immunogenicity. Toward the later stages of the smallpox erdication campaign, additional attenuated strains of W were developed and tested in humans. Of these, MVA has garnered substantial recent interest as potentially safer smallpox vaccine. Yet, MVA was never studied in a smallpox endemic area, and its efficacy is unknown.

Currently available live-attenuated vaccinia (VV)-based preparations of smallpox vaccine are associated with high rates of adverse reactions and are not safe for use in immunodeficient individuals (e.g., those infected with HIV) or those with a variety of rather common medical conditions (e.g., pregnancy or eczema), development of vaccines that are substantially safer, but of equivalent or better immunogenicity (and protective efficacy as documented in a surrogate animal model system) than the currently available VV vaccine preparations is imperative. While certain attenuated strains of VV (especially MVA) have highly desirable safety features and impressive immunogenicity properties when used to express heterologous antigens, our recent studies of the biology of MVA vaccination, along with published data from the comparative immunogenicity of replication-competent W strains versus MVA obtained by others, suggest that MVA may be an insufficiently immunogenic vaccine to reliably engender protective responses against variola or other highly pathogenic orthopoxviruses. Because MVA infection of DCs is abortive prior to the expression of late viral gene products (including viral structural proteins that are major targets that elicit neutralizing antibody responses), the level of induction of anti-MVA neutralizing antibodies (while sufficient to interfere with our ability to use the same MVA-based vaccine vector in sequential prime/boost immunizations) may be insufficient to provide cross-protection against infection with variola.

In one embodiment MVA is modified such that key late viral gene products that are targets for protective immune responses are expressed early in the virus life cycle, thereby promoting increased presentation of these antigens by DCs and consequently, the elicitation of higher level, more effective immune responses that are directed against the vector. It must be noted that this approach to increase the neutralizing immune responses that are directed against MVA for successful use as a vaccine against smallpox stands directly opposite to that of the previously described ‘deletion-mutant’ approach to vaccine vectors wherein MVA-specific neutralizing responses were diminished to allow greater utility of that vaccine vector for immunization against heterologous (non-poxvirus) antigens. The following modifications preserve the desirable safety characteristics of MVA, but confer a substantially enhanced ability to raise durable, high level cellular and humoral immune responses that are cross-reactive with major pathogenic orthopoxviruses.

To enhance the immunogenicity of a MVA vaccine against smallpox, VV genes B5R, A33R, L1R, and A27L have been cloned from the NYCBH vaccine strain and expressed from strong early (mH5) promoters that have been recombined into MVA sites II and III. The intracellular mature virion (IMV) constituents L1R, A27L and the extracellular enveloped virion (EEV) surface protein B5R are well-described targets of VV neutralizing antibodies. Although antibodies against EEV A33R component do not neutralize VV in culture, they do play a protective role In vivo in model antigens. Vaccination with these VV subunits, alone and in combination, induce protection against virulent challenge in mice and induce VV neutralizing antibodies in macaques. Because expression of EEV and IMV proteins in isolation can result in improper trafficking (and perhaps conformation), soluble secreted versions of these EEV and IMV antigens were expressed by mutant genes encoding solely the extracellular domains (that are targeted by neutralizing antibodies). In this way, both proper folding and trafficking can be accomplished, as well as potentially increased MHC Class II presentation of these relevant VV antigens.

2.9 Multivalent MVA-based Vaccine Vectors

Another embodiment provides multivalent vaccines (that simultaneously engender effective immunization against more than one infectious agent) by inclusion of genetic cassettes that direct the expression of relevant (protective) antigens from other organisms that cause infectious disease. In the developed world, use of a single multivalent MVA-based vaccine that replaces several currently-licensed vaccines would have the practical benefit of significantly reducing both the overall costs of vaccine production and the complexity of current immunization schedules. In developing nations where childhood diseases such as measles and polio have not been eradicated (and are associated with high mortality rates [e.g., 5-15% due to measles]), the use of multivalent MVA vaccines holds the promise to provide enormous public health benefits not otherwise achievable with currently available vaccines.

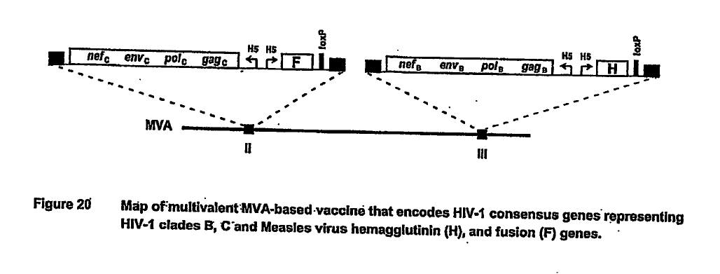

For example, successful immunization against measles virus with current live-attenuated or inactivated vaccines is dependent upon the recipient possessing low titers of measles-specific maternal antibodies (that wane between 9-15 months of age) to prevent neutralization of the vaccine itself. In geographical areas where measles is endemic, such conditions of low-level maternal antibodies against measles virus that which are required for successful immunization simultaneously constitute a window of susceptibility for pathogenic infection by circulating strains of measles virus, Because routine immunization against smallpox (that generates significant titers of VV-neutralizing antibodies) was discontinued a generation ago, maternal antibodies against VV (and MVA) would not interfere with the early successful delivery (at 9 months of age) of a multivalent MVA-based vaccine that encodes measles virus antigens as one of its valencies. Such an immunization would be expected to prime measles-specific cellular and humoral immune responses (that could in turn be augmented via booster immunization with rMVA or conventional measles virus vaccines), if not confer lasting protection against measles. In one embodiment, a multivalent MVA-based vaccine that encodes measles virus antigens in addition to its inclusion of HIV-1 genes (FIG. 20) possesses certain potential advantages (as described above) for use in developing nations. Because there is great capacity ( 30 kilobases) for expression of foreign genes from the disclosed recombinant MVA vectors, embodiments include genes of the following pathogens for combinatorial inclusion in multivalent MVA-based vaccines: measles, polio, mumps, rubella, HepA, HepB, HepC, influenza, VSV, VZV, HSV, EBV, HCMV, HHV-6, HHV-7, KSHV, YFV, West Nile Virus, plasmodium, mycobacterium, and SARS. This list is not exhaustive, and the methods and compositions of the present disclosure contemplate incorporation of antigens from any infectious disease. Furthermore, the methods and compositions of the present invention may be useful in the treatment of other diseases, in particular cancer.

2.10 Administration

Embodiments of the disclosure include pharmaceutical compositions such as vaccine compositions and dosage forms comprising the disclosed MVAs and vectors, or a pharmaceutically accept salt or prodrug thereof. Pharmaceutical compositions and unit dosage forms of the disclosure typically also comprise one or more pharmaceutically acceptable excipients or diluents. Advantages provided by specific compounds of the disclosure, such as, but not limited to, increased solubility and/or enhanced flow, purity, or stability (e.g., hygroscopicity) characteristics can make them better suited for pharmaceutical formulation and/or administration to patients than the prior art.

Pharmaceutical unit dosage forms of the compounds of this disclosure are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure will typically vary depending on their use. For example, a dosage form used in the acute treatment of a disease or disorder may contain larger amounts of the active ingredient, for example a MVA, than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this disclosure will vary from one another will be readily apparent to those skilled in the art. See, e.g., Remington's Pharmaceutical Sciences, 18th ed., Mack Publishing, Easton, Pa. (1990).

Typical pharmaceutical compositions and dosage forms comprise one or more excipients. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that comprise primary or secondary amines are particularly susceptible to such accelerated decomposition.

The disclosure further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate or organic acids. A specific solubility modulator is tartaric acid.

Like the amounts and types of excipients, the amounts and specific type of active ingredient in a dosage form may differ depending on factors such as, but not limited to, the route by which it is to be administered to patients. However, typical dosage forms of the compounds of the disclosure comprise a pharmaceutically acceptable salt of a disclosed MVA, or a pharmaceutically acceptable polymorph, solvate, hydrate, dehydrate, co-crystal, anhydrous, or amorphous form thereof, in an amount of from about 10 mg to about 1000 mg, preferably in an amount of from about 25 mg to about 750 mg, and more preferably in an amount of from 50 mg to 500 mg.

### EXAMPLES

### Example 1

### Cells

The UMNSAH/DF-1 chicken embryo fibroblast cell line (‘DF-1’), kindly provided by H. Varmus (Memorial Sloan-Kettering Cancer Center, New York, N.Y.) and currently available through ATCC (Manassas, Va.), was propagated in Dulbecco's Modified Eagle's Medium (DMEM) that was supplemented with 10% heat-inactivated fetal bovine serum (FBS; HyClone, Logan, Utah), 100 I.U./ml penicillin (PEN), 100 μg/ml streptomycin (STREP), and 2 mM L-glutamine (GLUT). Primary chicken embryo fibroblasts (CEF) prepared from 8-11 day embryos were obtained from Chales River SPAFAS, Inc. (Preston, Conn.) and propagated in Basal Medium Eagle that was supplemented with 5% FBS, 100 I.U./ml PEN, 100 μg/ml STREP, and 2 mM GLUT. All DF-1-derived cell lines (described below) were propagated in DF-1 growth medium that was supplemented with 300 μg/ml G418 Sulfate. All tissue culture growth media and supplements were obtained from Mediatech (Hemdon, Va.) unless noted otherwise. Zeocin was purchased from Invitrogen (Carlsbad, Calif.).

### Example 2

### Generation of DF-1-derived Cell Lines

To allow generation of DF-1-derived cell lines that constitutively express UGD^{MVA} or CRE recombinase, the pCAN gene-expression vector was constructed for use in avian cells by subcloning a 1.7 kb CMV IE-chicken β-Actin promoter/enhancer element (kindly provided by J. Jacob, Emory Vaccine Center) into pNEB193 (New England Biolabs, Beverly, Mass.) to yield pCMVACT193. Subsequently, a 2.3 kb BamHl SV40-Neo^{R} expression cassette was subcloned from pIRES (BD Biosciences Clontech, Palo Alto, Calif.) into pCMVACT to generate pCAN (CMV IE-chicken β-Actin/Neo^{R}).

The udg ORF (MVA nucleotides 92,417-93,073; Genbank accession U94848) was amplified via polymerase chain reaction from genomic MVA DNA with forward primer 5′-tctcgagctcaATGAATTCAGTGACTGTATCA-3′ (SEQ ID NO. 67) (initiator methionine codon underlined) and reverse primer=5′-cgcggtaccgtcTTAATAAATAAACCCTTGAGC-3′ (SEQ ID NO. 68) (stop codon underlined; udg ORF in capitals) and cloned into pCR2.1 (Invitrogen) to yield p2.1udgORF. The udg ORF was subsequently re-amplified via PCR with forward primer 5′-aaagcftagatctgccaccATGAATTCAGTGACTGTA-3′ (SEQ ID NO. 69)(Kozak consensus in bold, initiator methionine codon underlined) and reverse primer 5′-agcggccgctacgtaTTAATAAATAAACCCTTG-3′ (SEQ ID NO. 70)(stop codon underlined) to incorporate a translation initiation consensus sequence immediately preceding the udg ORF. This PCR product was cloned into the pCR-Blunt II-TOPO vector (Invitrogen) to yield pDG100, its integrity confirmed via DNA sequencing, and was subsequently subcloned under the control of the CMV IE-chicken β-Actin promoter/enhancer element in the PCAN expression vector to yield pCANudg.

DF-1-derived cell lines that constitutively express UGD^{MVA} were generated by calcium phosphate-mediated transfection of the udg-expression plasmid pCANudg into DF-1 cells followed by clonal selection of G418^{R} cells. G418^{R} cell lines were screened for their ability to complement the growth of ts4149, a vaccinia virus mutant that harbors a temperature-sensitive mutation in the udg (D4R) gene (generously provided by G. McFadden, University of Western Ontario, Canada), at the non-permissive temperature of 39.5° C. The G418^{R} cell lines that exhibited the highest levels of complementation, designated CAN20 and CAN17 were subsequently used to generate and propagate udg-deletion recombinants of MVA.

To generate a CRE recombinase-expression vector, the cre ORF was amplified via PCR from pBS185 (Gibco BRL) with forward primer 5′-aagcttagatctgccaccATGTCCAATTTTACTGACC-3′ (SEQ ID NO. 71) (initiator met codon underlined) and reverse primer 5′-gtttaaacgcggccgcCTATTCTAGTGTTAGTGATGCTAGTGGTGATGGTAGTGTTACATCGCCATCTTCCAG-3′ (SEQ ID NO. 72) (stop codon in bold, NES1-encoding sequence underlined) to generate a C-terminal fusion between CRE and the nuclear export signal (N-VTLPSPLASLTLE-C, NES1 (SEQ ID NO. 73) of EBV. The cre-NES1 PCR product was cloned into pCR-Blunt II-TOPO vector (Invitrogen, Carlsbad, Calif.) to yield pCRE-TOPOM2, confirmed via DNA sequencing, and subsequently cloned under the control of the CMV IE-chicken β-Actin promoter/enhancer element in pCAN to yield the CRE expression vector pCANcre. Following calcium phosphate-mediated transfection of DF-1 cells with pCANcre, G418^{R} cell clones were isolated and screened for their ability to mediate excision of a loxP-flanked pH5-gfpzeo cassette from rMVA.

### Example 3

### Viruses

MVA (p579), generously provided by B. Moss (National Institutes of Health), was amplified on primary CEFs or DF-1 chicken embryo fibroblasts as indicated. Virus stocks were prepared as lysates of infected cells that were subsequently clarified via centrifugation (800 g). Infectious titers of virus stocks were determined via TCID₅₀ assay on primary CEFs (where indicated) or via plaque assay on DF-1 cell monolayers. For immunization experiments, rMVAs were purified via sedimentation through a 36% sucrose cushion.

### Example 4

### Generation and Isolation of Recombinant MVAs

Recombinant MVAs encoding GFPZEO^{R} were generated via homologous recombination by infecting 2×10⁶ permissive cells at MOI=0.05 for 1.5 hours followed by transfection of 1 μg MVA transfer vector (supercoiled plasmid DNA) via Effectene (Qiagen, Valencia, Calif.) according to the manufacturer's protocol. At 48 hours following infection, progeny viruses were released from infected cells via lysis (1 freeze/thaw cycle followed by sonication) and were plated at various dilutions onto monolayers of permissive cells (DF-1 or DF-1-derivatives). gfpzeo⁺ recombinant viruses were selected for by application of an agarose overlay (1% low-melting agarose/1× DMEM (GibcoBRL)) that was supplemented with 2% FBS, 100 I.U./ml PEN, 100 μg/ml STREP, and 200 μg/ml Zeocin (Invitrogen). Recombinant viruses were identified as foci of GFP⁺ cells that were readily detected microscopically by 2 days following infection. Recombinant viruses were plaque purified through at least 3 rounds of Zeocin selection and analyzed by diagnostic Southern blots to ensure clonality.

### Example 5

### BcIXL-inhibition of MVA-induced Apoptosis

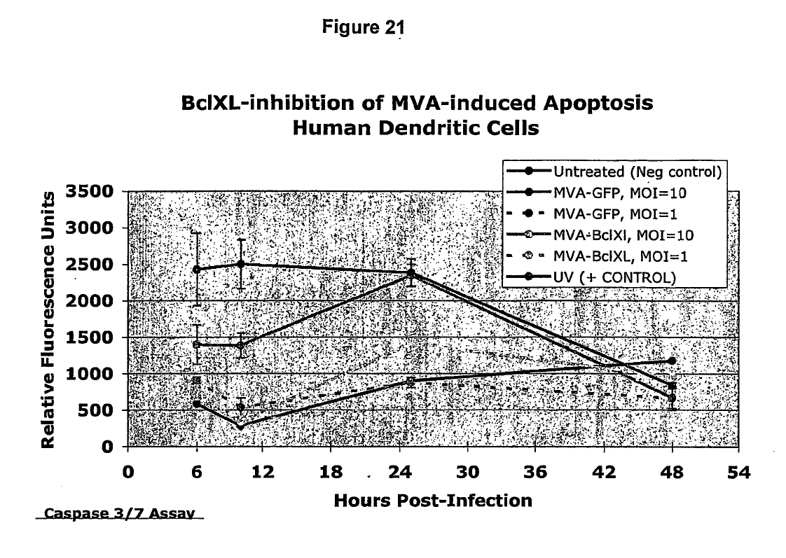

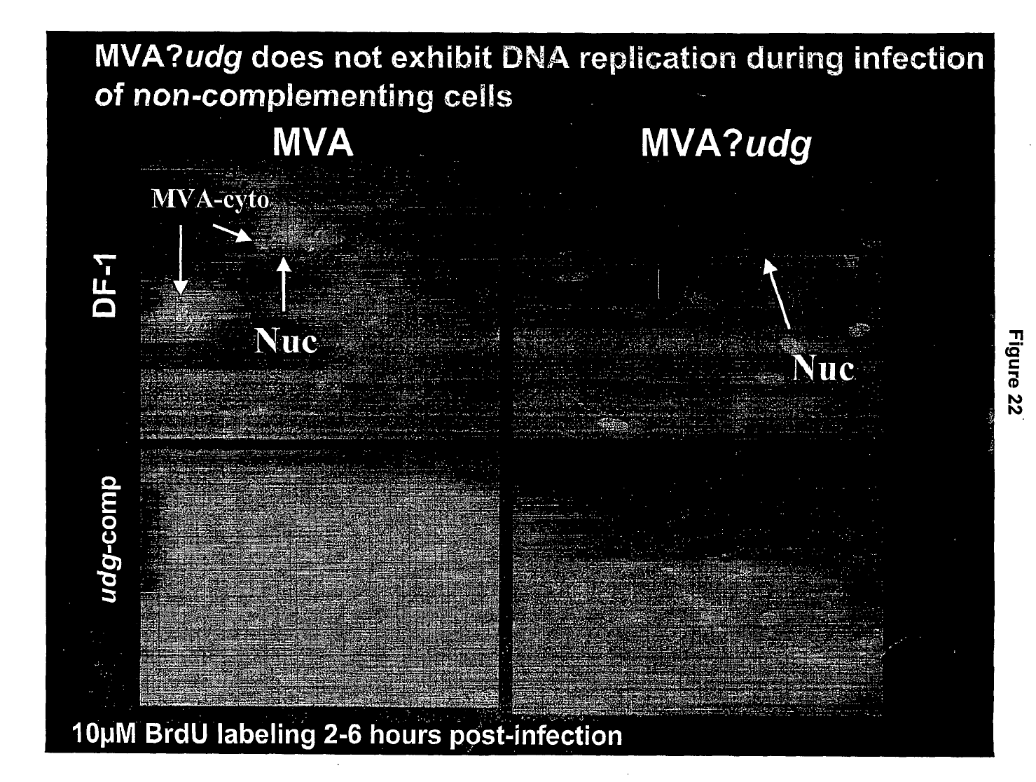

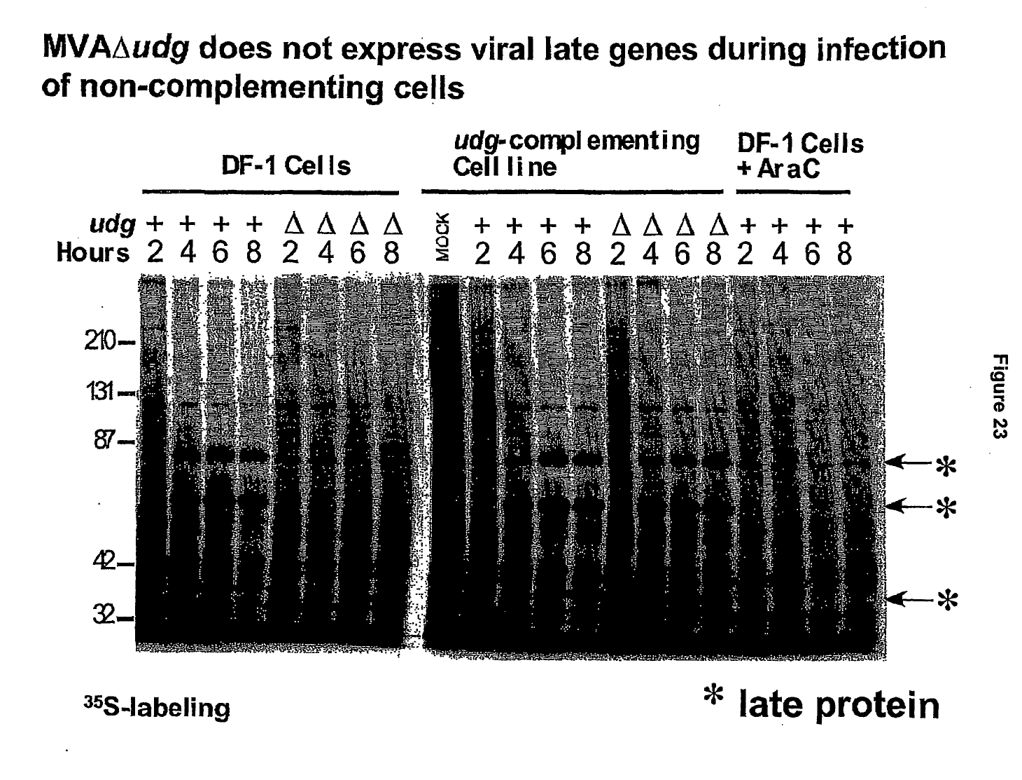

Human monocyte-derived dendritic cells were infected, as indicated with MVA-GFP or MVA-BcIXI in FIG. 21. Levels of caspase-3/7 activity, an indicator of cellular apoptosis, were measured in DCs by the detection of fluorescence emitted following enzymatic cleavage of a fluorescent (DEVD-AmC) caspase-3/7 substrate.

### Example 6

### MVAΔudg Does Not Exhibit DNA Replication During Infection of Non-Complementing Cells

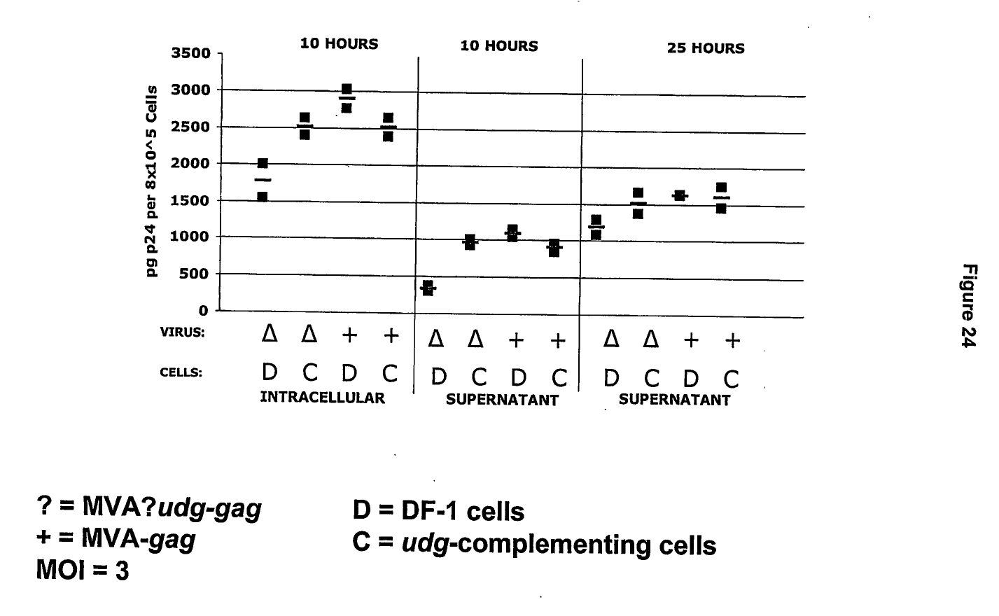

DF-1 fibroblasts (DF-1) or a UDG-complementing DF-1-derived cell line (udg-comp) were infected with MVA at a MOI=3 (FIG. 22). Infected cells were labelled with BrdU during the interval of 2-6 hours post-infection. BrdU that was incorporated into newly replicated DNA was detected via immunofluorescent detection with anti-BrdU-FITC antibody. This technique labels newly synthesized cellular DNA within nuclei (Nuc) and newly synthesized viral DNA in the cytoplasm (MVA-cyto). These data show that the recombinant MVA constructs disclosed herein do not replication in non-complementing cells.

### Example 7

### MVA udg Does Not Express Viral Late Genes During Infection of Non-Complementing Cells In Culture.

DF-1 and udg-complementing (DF-1-derived) cells were infected with MVA (udg+) or MVA UDG ( ) at MOI=10 in the absence or presence of 150 μM AraC, as indicated in FIG. 23. Infected cell proteins were metabolically labeled with 35S-methionine for 30 min immediately prior to harvesting at indicated times post infection. Proteins were separated via SDS-PAGE and visualized by autoradiography. Arrows denote viral late gene products. As can be seen in FIG. 23, MVA udg does not express viral late genes during infection of non-complementing cells in culture

### Example 8

### HIV-Gag Expression is Comparable During Infection with MVAΔudg-gag and MVA-gag

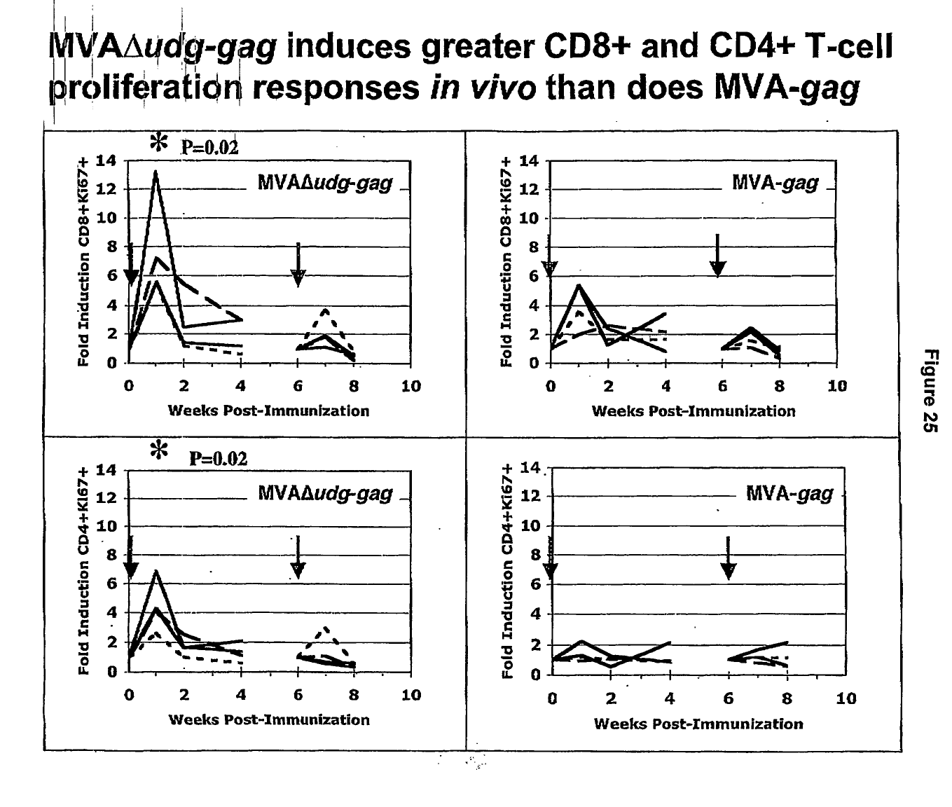

DF-1 fibroblasts and CAN20 cells (UDG-complementing DF1-derived cells) were infected with MVA-gag or MVA udg-gag at MOI=3 (FIG. 24). Culture supernatants (supernatant) or cell lysates (intracellular) were assayed via HIV p24Gag ELISA at 10, 25 hours following infection to quantify levels of HIV Gag antigen. HIV p24Gag ELISAs were purchased from NCI and used according to manufacturer's protocols. These data show that heterologous nucleic acid sequences of the disclosed rMVA vectors express antigenic polypeptides.

### Example 9

### MVA UDG-gag Immunization Induces Greater CD8+ and CD4+ T-cell Proliferation Responses In Vivo Than Does MVA-gag

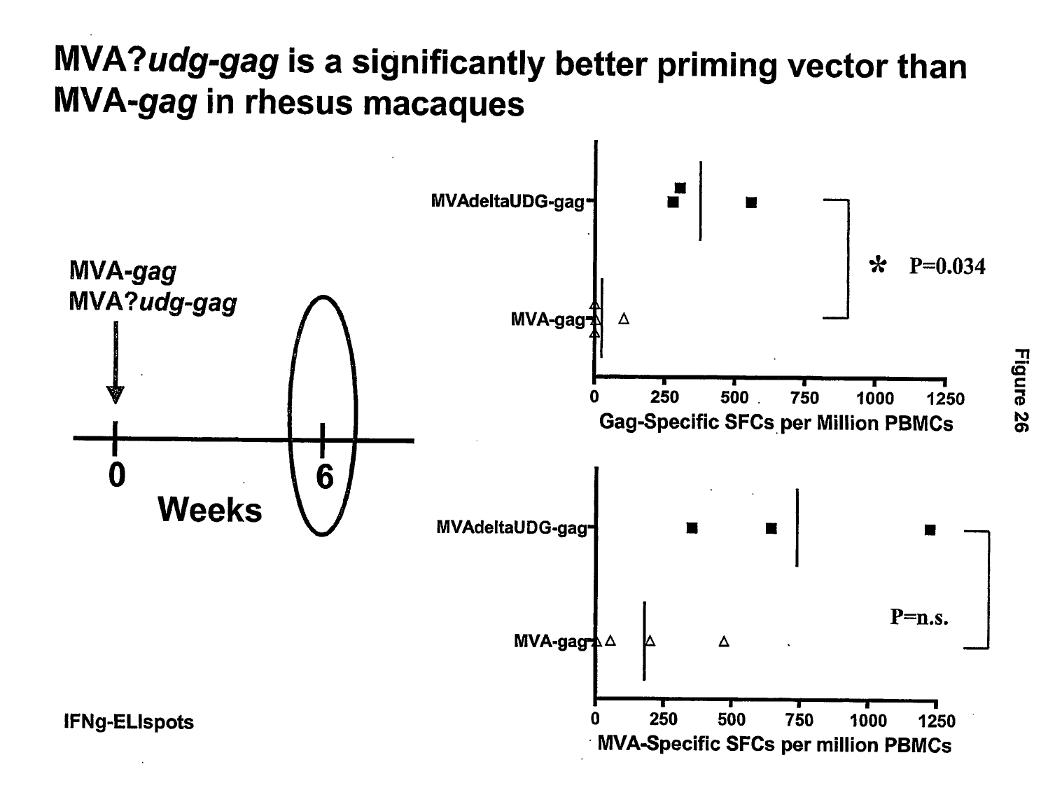

Lymphocytes in whole blood samples obtained from rhesus macaques at indicated times following immunization with MVA-gag or MVA udg-gag viruses were stained with fluorescently labelled antibodies (Becton-Dickinson) for cell surface expression of CD3, CD4, CD8, intracellular expression of Ki67, and analyzed via flow cytometry on a FACS calibur (FIG. 25). (Antibodies used for flow cytometric staining are of anti-human antigen specificity and have been previously verified to cross react with rhesus macaque antigens). Absolute numbers of Ki67-positive CD4+ and CD8+ T-cells per pl of blood were calculated at each timepoint. Values plotted from weeks 0 through 4 have been normalized against each individual macaque's pre-immunization baseline values. The values plotted for weeks 6 through 8 have been normalized to each macaque's level at week 6 (time of boost). Immunization with MVA UDG-gag resulted in significantly higher peak induction of CD8+ and CD4+ T-cell proliferation (p=0.02, Mann-Whitney, I week post-immunization). MVA UDG-gag immunization induces greater CD8+ and CD4+ T-cell proliferation responses in vivo than does MVA-gag.

### Example 10

### MVA UDG-gag Elicits Significantly Higher Gag-specific Cellular Immune Responses Than MVA-gag Following Single Dose Immunization of Rhesus Macaques

Antigen-specific cellular immune responses were assayed via IFNg-ELIspot analysis of PBMCs at 6 weeks following immunization of macaques with 2×10ˆ8 PFU of indicated rMVA (1×10ˆ8 PFU intradermally+1×10ˆ8 PFU intramuscularly). Gag-specific cellular responses (SFCs, spot-forming cells) were measured following in vitro stimulation of PBMCs with pools of overlapping Gag peptides (15mers, overlapping by 11). MVA-specific cellular immune responses were measured following in vitro stimulation of PBMCs with MVA. Gag-specific responses following immunization with MV UDG-gag were significantly higher than those observed following immunization with MVA-gag (p=0.034, Mann-Whitney). The difference in MVA-specific responses between groups does not achieve statistical significance. SFCs=spot-forming cells. The data are provided in FIG. 26 and confirm that MVA UDG-gag elicits significantly higher Gag-specific cellular immune responses than MVA-gag following single dose immunization of rhesus macaques.

### Example 11

### MVAΔudg-gag Elicits Higher Levels of Gag-specific Cellular Memory Immune Responses Than MVA(udg+)-gag in Macaques

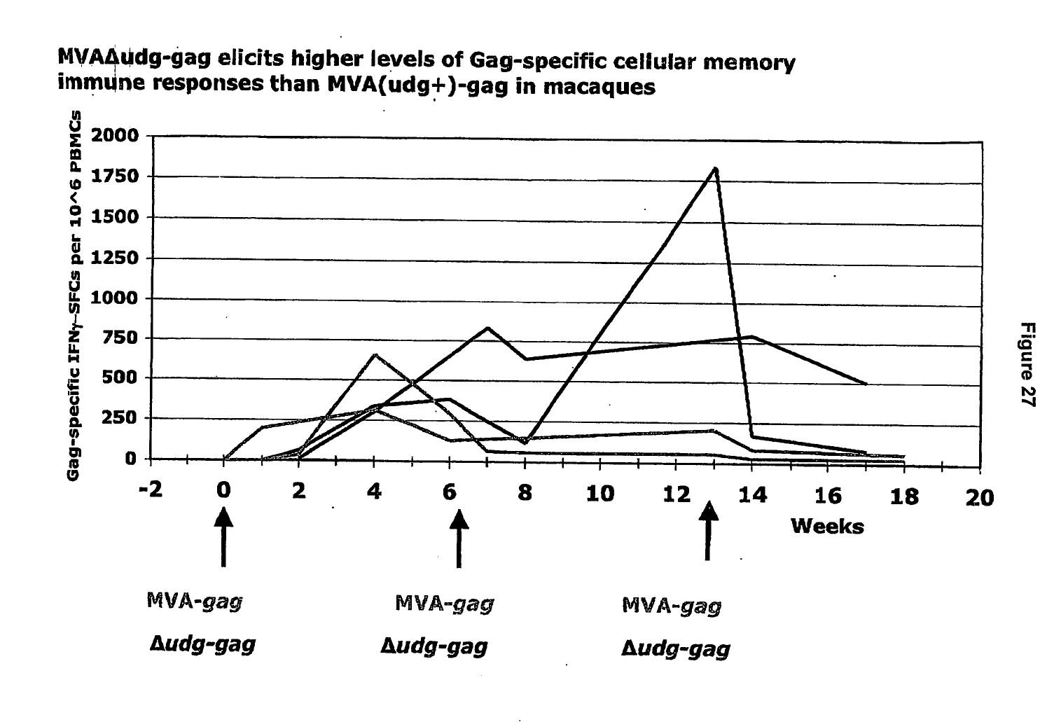

Gag-specific cellular immune responses were determined via IFN gamma ELlspot analysis of PBMCs from rhesus macaques following immunization with 2×10ˆ8 PFU of rMVA, as indicated in FIG. 27. Gag-specific cellular responses (SFCs, spot-forming cells) were measured via IFNgamma ELlspot assay following in vitro stimulation of PBMCs with pools of overlapping HIV subtype B consensus Gag peptides (15mers, overlapping by 11) that were obtained from the NIH AIDS Reagent Repository.

### Example 12

### MVAΔudg-gag Elicits Higher Levels of Cellular Immune Responses than MVA(udg+)-gag Following Single-Dose Immunization of Rhesus Macaques

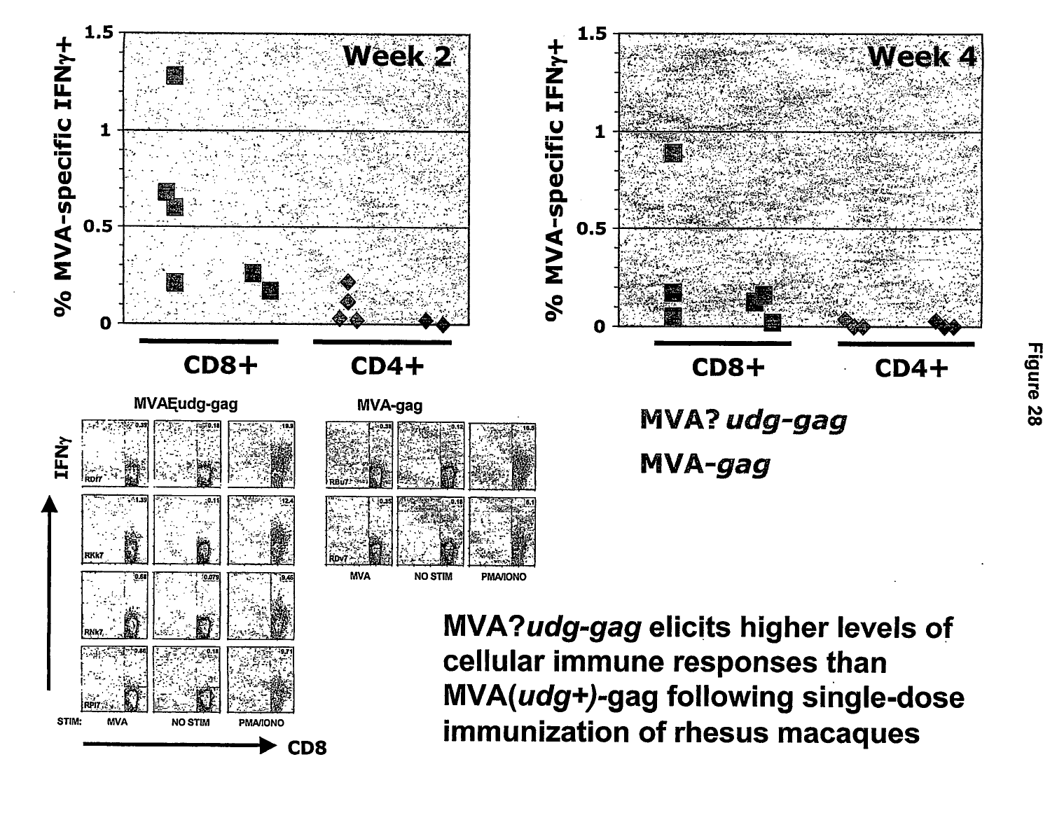

In FIG. 28, frequencies of MVA-specific CD8+ and CD4+ T cells were determined in PBMCs isolate at 2, 4 weeks following immunization of rhesus macaques with MVA-gag or MVA udg-gag (2×10ˆ8 PFU/macaque) via intracellular cytokine staining (IFNY) following overnight stimulation of rhesus macaque PBMCs with MVA (MOI=2).

### Example 13

### Working Model Explaining Increased Immunogenicity Exhibited by the udg-Deletion rMVAs

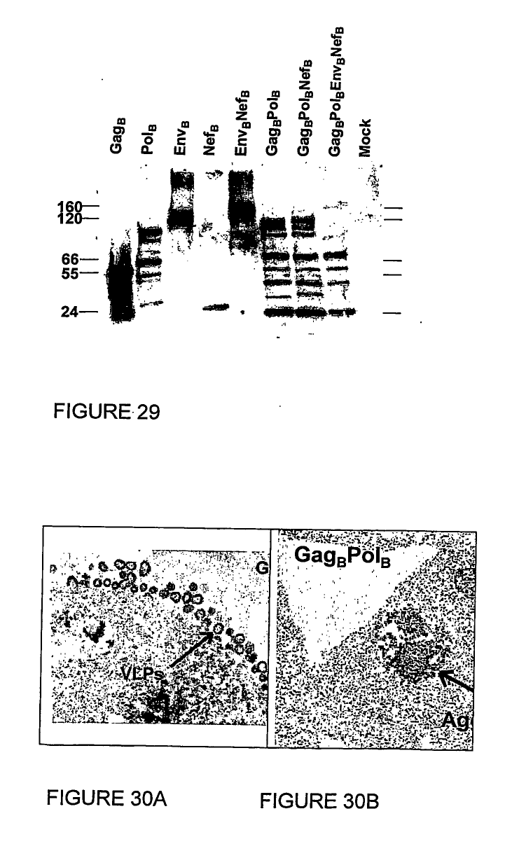

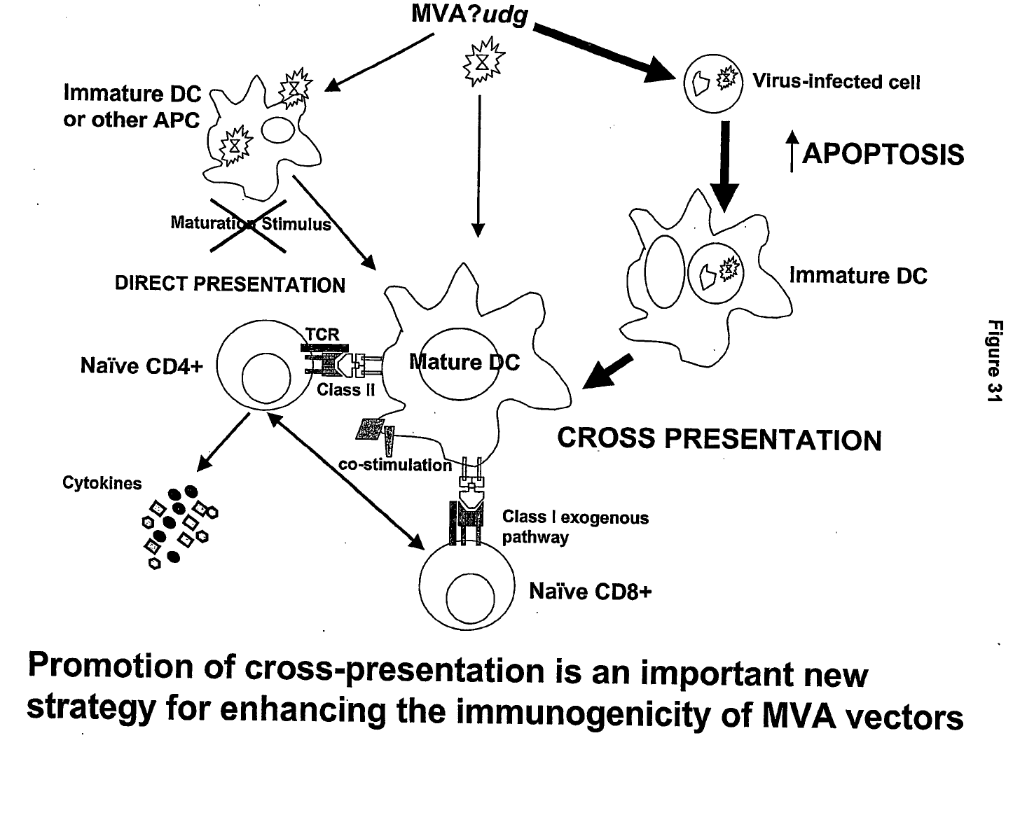

While both udg+ and udg rMVAs induce apoptosis of infected immature dendritic cells, only the udg mutant induces apoptosis in non-APCs (such as fibroblasts at the site of immunization). The active death of these infected cells, in turn, constitutes a potent source of antigens for uptake by un-infected DCs that facilitates cross-presentation of MVA-encoded antigens to elicit cellular immune responses (FIG. 29).

### Example 14

### CRE Recombinase-mediated Insertion of a loxP/GFP-expression Cassette into the MVA Genome

An rMVA that harbors a single loxP sequence in place of the udg ORF at the udg locus was grown in UDG-complementing cells that were transiently transfected with a CRE-expression plasmid(=) or salmon sperm DNA (−). CRE-mediated insertion of gfpzeo was assessed by scoring GFP+ plaques.

| | GFP-negative | GFP-positive | Total | % |
|---|---|---|---|---|
| CRE | plaques | plaques | plaques | Recombnant |
| + | 1036 | 7 | 1043 | 0.67 |
| + | 1188 | 9 | 1197 | 0.75 |
| + | 1014 | 7 | 1021 | 0.69 |
| − | 1058 | 0 | 1058 | 0 |
| − | 1074 | 0 | 1074 | 0 |
| − | 1032 | 0 | 1032 | 0 |

It should be emphasized that the above-described embodiments of the present disclosure, particularly, any “preferred” embodiments, are merely possible examples of implementations, merely set forth for a clear understanding of the principles of the disclosed subject matter. Many variations and modifications may be made to the above-described embodiment(s) without departing substantially from the spirit and principles of the disclosure. All such modifications and variations are intended to be included herein within the scope of this disclosure and protected by the following claims.

We claim:

1. A system for producing recombinant modified vaccinia Ankara virus comprising:
an immortalized, non-transformed avian fibroblast cell infected with a recombinant modified vaccinia Ankara virus comprising a first null mutation in a vaccinia gene necessary for replication of the recombinant modified vaccinia Ankara virus, wherein the cell is engineered to express the vaccinia gene necessary for viral replication to enable the recombinant modified vaccinia Ankara virus to replicate in the cell.

2. The system of claim 1, wherein the cell is a chicken embryo fibroblast.

3. The system of claim 1, wherein the cell is a DF-1 cell.

4. The system of claim 1, wherein the gene necessary for viral replication is vaccinia uracil DNA glycosylase.

5. The system of claim 1, wherein the null mutation comprises a deletion.

6. The system of claim 1, wherein the modified vaccinia Ankara virus comprises a first heterologous nucleic acid sequence.

7. The system of claim 6, wherein the first heterologous nucleic acid sequence encodes a first antigen.

8. The system of claim 7, wherein the first antigen is selected from the group consisting of an HIV antigen, measles virus antigen, polio virus antigen, mumps virus antigen, rubella virus antigen, hepatitis virus antigen, SARS virus antigen, influenza virus antigen, herpes virus antigen, West Nile Virus antigen, malaria plasmodium antigen, tuberculosis bacillus antigen, yellow fever virus antigen, dengue flavivirus antigen, river blindness nematode antigen, Epstein-Barr virus antigen, and combinations thereof.

9. The system of claim 8, wherein the HIV antigen comprises an optimized consensus sequence of HIV subtype A, B, or C polypeptides selected from the group consisting of Pol, Gag, Env, Nef, combinations thereof, a fusion polypeptide thereof, and fragments thereof.

10. The system of claim 7, wherein the modified vaccinia Ankara virus comprises a second heterologous nucleic acid sequence operably linked to an early stage viral promoter.

11. The system of claim 10, wherein the second heterologous nucleic acid sequence encodes a pro-apoptotic, an anti-apoptotic factor, or a fragment thereof.

12. The system of claim 11, wherein the pro-apoptotic factor is selected from the group consisting of Bax, Bak, Bid, Fas receptor, AIF, caspase 3-CPP32, fragments thereof, and combinations thereof.

13. The system of claim 11, wherein the anti-apoptotic factor is selected from the group consisting of Bcl2, Bcl-xl, Ciapl, Ciap2, Flame, CrmA, p35, Xiap, MC159, a fragment thereof, and combinations thereof.

14. The system of claim 11, wherein the modified vaccinia Ankara virus comprises a third heterologous nucleic acid sequence operably linked to an early stage viral promoter.

15. The system of claim 14, wherein the third heterologous nucleic acid sequence encodes an immunomodulator.

16. The system of claim 15, wherein the immunomodulator is selected from the group consisting of GM-CSF, IL-15, MIP3alpha, fragments thereof, and combinations thereof.

17. The system of claim 14, wherein the modified vaccinia Ankara virus comprises a fourth heterologous nucleic acid sequence operably linked to an early stage viral promoter.

18. The system of claim 14, wherein the fourth heterologous nucleic acid sequence encodes a second antigen.

19. The system of claim 18, wherein the first antigen and the second antigen are from different viral subtypes.

20. The system of claim 18, wherein the first antigen and the second antigen are from different organisms.

21. The system of claim 1, further comprising a second null mutation in a gene selected from the group consisting of IL1 beta receptor, A46R, IL-18BP, A41L, and E3L.

22. An immortalized, non-transformed avian fibroblast cell infected with modified vaccinia Ankara virus.

23. The cell of claim 22, wherein the cell is engineered to express a gene necessary for vaccinia virus replication.

24. The cell of claim 22, wherein the modified vaccinia Ankara virus comprises a null mutation in the gene necessary for modified vaccinia Ankara virus replication.

25. The cell of claim 24, wherein the gene necessary for modified vaccinia Ankara virus replication is vaccinia uracil DNA glycosylase.

26. The cell of claim 24, wherein the null mutation comprises a deletion.

27. The cell of claim 24, wherein the modified vaccinia Ankara virus encodes a heterologous antigen.

28. The cell of claim 27, wherein the heterologous antigen is selected from the group consisting of an HIV antigen, measles virus antigen, polio virus antigen, mumps virus antigen, rubella virus antigen, hepatitis virus antigen, SARS virus antigen, influenza virus antigen, herpes virus antigen, West Nile Virus antigen, malaria plasmodium antigen, tuberculosis bacillus antigen, yellow fever virus antigen, dengue flavivirus antigen, river blindness nematode antigen, Epstein-Barr virus antigen, and combinations thereof.

29. The cell of claim 28, wherein the HIV antigen comprises an optimized consensus sequence of HIV subtype A, B, or C polypeptides selected from the group consisting of Pol, Gag, Env, Nef, combinations thereof, a fusion polypeptide thereof, and fragments thereof.

30. A recombinant modified vaccinia Ankara virus comprising a first null mutation in a vaccinia gene necessary for replication of the recombinant modified vaccinia Ankara virus.

31. The recombinant modified vaccinia Ankara virus of claim 30, wherein the virus is propagated in an immortalized, non-transformed avian fibroblast cell line engineered to complement the first null mutation.

32. The recombinant modified vaccinia Ankara virus of claim 31, wherein the cell line is a chicken embryo fibroblast cell line.

33. The recombinant modified vaccinia Ankara virus of claim 31, wherein the cell lines is DF-1.

34. The recombinant modified vaccinia Ankara virus of claim 30, wherein the virus comprises a first heterologous nucleic acid sequence.

35. The recombinant modified vaccinia Ankara virus of claim 34, wherein the first heterologous nucleic acid sequence encodes a first antigen.

36. The recombinant modified vaccinia Ankara virus of claim 35, wherein the first antigen is selected from the group consisting of an HIV antigen, measles virus antigen, polio virus antigen, mumps virus antigen, rubella virus antigen, hepatitis virus antigen, SARS virus antigen, influenza virus antigen, herpes virus antigen, West Nile Virus antigen, malaria plasmodium antigen, tuberculosis bacillus antigen, yellow fever virus antigen, dengue flavivirus antigen, river blindness nematode antigen, Epstein-Barr virus antigen, and combinations thereof.

37. The recombinant modified vaccinia Ankara virus of claim 36, wherein the HIV antigen comprises an optimized consensus sequence of Pol, Gag, Env, Nef, combinations thereof, a fusion polypeptide thereof, or a fragment thereof.

38. The recombinant modified vaccinia Ankara virus of claim 37, wherein the modified vaccinia Ankara virus comprises a second heterologous nucleic acid sequence operably linked to an early stage viral promoter.

39. The recombinant modified vaccinia Ankara virus of claim 38, wherein the second heterologous nucleic acid sequence encodes a pro-apoptotic, an anti-apoptotic factor, an immunomodulator, a second antigen, or fragments thereof.

40. The recombinant modified vaccinia Ankara virus of claim 39, wherein the pro-apoptotic factor is selected from the group consisting of Bax, Bak, Bid, Fas receptor, AIF, caspase 3-CPP32, fragments thereof, and combinations thereof.

41. The recombinant modified vaccinia Ankara virus of claim 39, wherein the anti-apoptotic factor is selected from the group consisting of Bcl2, Bcl-xl, Ciapl, Ciap2, Flame, CrmA, p35, Xiap, MC159, a fragment thereof, and combinations thereof.

42. The recombinant modified vaccinia Ankara virus of claim 39, wherein the immunomodulator is selected from the group consisting of GM-CSF, IL-15, MIP3alpha, fragments thereof, and combinations thereof.

43. The recombinant modified vaccinia Ankara virus of claim 39, wherein the first antigen and the second antigen are from different viral subtypes.

44. The recombinant modified vaccinia Ankara virus of claim 39, wherein the first antigen and the second antigen are from different organisms.

45. The recombinant modified vaccinia Ankara virus of claim 39, wherein the modified vaccinia Ankara virus comprises a third heterologous nucleic acid sequence operably linked to an early stage viral promoter.

46. The recombinant modified vaccinia Ankara virus of claim 42, wherein the third heterologous nucleic acid sequence encodes a second immunomodulator, a third antigen, or fragments thereof.

47. The recombinant modified vaccinia Ankara virus of claim 42, wherein the third heterologous nucleic acid sequence encodes a second pro-apoptotic factor if the second heterologous nucleic acid sequence encodes a first pro-apoptotic factor.

48. The recombinant modified vaccinia Ankara virus of claim 42, wherein the third heterologous nucleic acid sequence encodes a second anti-apoptotic factor if the second heterologous nucleic acid sequence encodes a first anti-apoptotic factor.

49. The recombinant modified vaccinia Ankara virus of claims 46-48, wherein the modified vaccinia Ankara virus comprises a fourth heterologous nucleic acid sequence operably linked to an early stage viral promoter.

50. The recombinant modified vaccinia Ankara virus of claim 49, wherein the fourth heterologous nucleic acid sequence encodes a fourth antigen.

51. The recombinant modified vaccinia Ankara virus of claim 35, further comprising a second null mutation in a gene selected from the group consisting of IL1-beta receptor, A46R, IL-18BP, A41L, and E3L.

52. A vaccine comprising:
a modified vaccinia Ankara virus comprising:
a null-mutation in a vaccinia viral gene necessary for repHcation of the modified vaccinia Ankara virus, and
a heterologous nucleic acid sequence encoding an antigen, and
a pharmaceutically acceptable excipient or carrier.

53. The vaccine of claim 52, wherein the viral gene necessary for viral replication is uracil DNA glycosylase.

54. The vaccine of claim 52, wherein the heterologous antigen is selected from the group consisting of an HIV antigen, measles virus antigen, polio virus antigen, mumps virus antigen, rubella virus antigen, hepatitis virus antigen, SARS virus antigen, influenza virus antigen, herpes virus antigen, West Nile Virus antigen, malaria plasmodium antigen, tuberculosis bacillus antigen, yellow fever virus antigen, dengue flavivirus antigen, river blindness nematode antigen, Epstein-Barr virus antigen, and combinations thereof.

55. The vaccine of claim 54, wherein the HIV antigen is an optimized consensus sequence of Pol, Gag, Env, Nef, combinations thereof, a fusion polypeptide thereof, or a fragment thereof.

56. The vaccine of claim 52, further comprising a second null mutation in gene selected from the group consisting of IL1 beta receptor, A46R, IL-18BP, A41L, and E3L

57. A method of propagating a modified vaccinia Ankara virus comprising:
culturing an immortalized, non-transformed cell engineered to express modified Ankara virus uracil DNA glycosylase; and
infecting the cell with a recombinant modified vaccinia Ankara virus, wherein the recombinant modified vaccinia Ankara virus cannot express functional uracil DNA glycosylase.

58. The method of claim 57, wherein the recombinant modified vaccinia Ankara virus encodes a heterologous antigen.

59. The method claim 58, wherein the heterologous antigen is selected from the group consisting of an HIV antigen, measles virus antigen, polio virus antigen, mumps virus antigen, rubella virus antigen, hepatitis virus antigen, SARS virus antigen, influenza virus antigen, herpes virus antigen, West Nile Virus antigen, malaria plasmodium antigen, tuberculosis bacillus antigen, yellow fever virus antigen, dengue flavivirus antigen, river blindness nematode antigen, Epstein-Barr virus antigen, and combinations thereof.

60. The method of claim 59, wherein the HIV antigen comprises an optimized consensus sequence of Pol, Gag, Env, Nef, combinations thereof, a fusion polypeptide thereof, or a fragment thereof.

61. The method of claim 57, wherein the cell is a chicken embryo fibroblast.

62. The method of claim 61, wherein the cell is a DF-1 cell.

63. The method of claim 57, further comprising the step of plaque purifying the recombinant modified vaccinia Ankara virus from a plurality of infected cells engineered to express modified Ankara virus uracil DNA glycosylase.

64. A recombinant modified vaccinia Ankara virus produced by the method of claim 57.

65. The recombinant modified vaccinia Ankara virus of claim 64, further comprising comprising a second null mutation in gene selected from the group consisting of IL1 beta receptor, A46R, IL-18BP, A41L, and E3L.

66. A smallpox vaccine comprising:
a recombinant modified vaccinia Ankara virus comprising a null mutation in a gene necessary for replication of the recombinant modified vaccinia Ankara virus, and one or more nucleic acid sequences operably linked to an early stage viral promoter, wherein the one more nucleic acid sequences encode one or more genes selected from the group consisting of B5R, A33R, L1R, A27L, and fragments thereof.

67. The smallpox vaccine of claim 66, wherein the gene necessary for replication of the recombinant modified vaccinia Ankara virus is vaccinia uracil DNA glycosylase.

68. The smallpox vaccine of claim 66, further comprising a second null mutation in gene selected from the group consisting of IL1 beta receptor, A46R, IL-18BP, A41L, and E3L.

69. A modified vaccinia Ankara virus comprising:
a null mutation in a vaccinia viral gene necessary for replication of the modified vaccinia Ankara virus, and
one to four heterologous nucleic acid sequences independently selected from the group consisting of SEQ ID NOs. 18-65, a heterologous antigen, a nucleic acid sequence encoding a pro-apoptotic factor, a nucleic acid sequence encoding an anti-apoptotic factor, a nucleic acid sequence encoding an immunomodulator, fragments thereof, or combinations thereof.

70. The modified vaccinia Ankara virus of claim 69, wherein the gene necessary for replication of the recombinant modified vaccinia Ankara virus is vaccinia uracil DNA glycosylase.

71. The modified vaccinia Ankara virus of claim 69, further comprising a second null mutation in gene selected from the group consisting of IL1 beta receptor, A46R, IL-18BP, A41L, and E3L.

72. A recombinant avian fibroblast cell engineered to constitutively express vaccinia uracil DNA glycosylase.

73. A method for vaccinating a host comprising,
administering a composition according to any one of claims 30-56 and 65-71 in an amount sufficient to affect an immune response in the host.

74. The method of claim 73, where in the host is a mammal.

75. A modified vaccinia Ankara virus comprising:
a heterologous nucleic acid sequence operably linked to a promoter, wherein the heterologous nucleic acid sequence encodes a pro-apoptotic factor, an anti-apoptotic factor, an immunomodulator, combinations thereof, or a fragment thereof.

76. The modified vaccinia Ankara virus of claim 75, further comprising a heterologous nucleic acid sequence encoding an antigen.

77. A modified vaccinia Ankara virus comprising:
a first heterologous nucleic acid sequence operably linked to a promoter, wherein the first heterologous nucleic acid sequence encodes a pro-apoptotic factor or a fragment thereof; and
a second heterologous nucleic acid operably linked to a promoter, wherein the second heterologous nucleic acid encodes an immunomodulator or a fragment thereof.

78. The modified vaccinia Ankara virus of claim 77, further comprising a third heterologous nucleic acid sequence operably linked to a promoter, wherein the third heterologous nucleic acid sequence encodes an antigen.

79. A modified vaccinia Ankara virus comprising:
a first heterologous nucleic acid sequence operably linked to a promoter, wherein the first heterologous nucleic acid sequence encodes an anti-apoptotic factor or a fragment thereof; and
a second heterologous nucleic acid operably linked to a promoter, wherein the second heterologous nucleic acid encodes an immunomodulator or a fragment thereof.

80. The modified vaccinia Ankara virus of claim 79, further comprising a third heterologous nucleic acid sequence operably linked to a promoter, wherein the third heterologous nucleic acid sequence encodes an antigen.

81. The modified vaccinia Ankara virus of claims 75-80 further comprising a null mutation in a gene necessary for replication of the modified Ankara virus.

82. The modified vaccinia Ankara virus of claims 75-81, further comprising a second null mutation in a gene selected from the group consisting of IL1 beta receptor, A46R, IL-18BP, A41L, and E3L.

83. A vaccine comprising the modified vaccinia Ankara virus of claims 81 or 82.

84. The vaccine of claim 83, further comprising a pharmaceutically acceptable excipient.

85. A method of vaccinating a host comprising administering the vaccine of claims 83 or 84 to host.

86. The method of claim 85, wherein the vaccine is administered in an amount sufficient to modulate an immune response in a host.

87. A vector comprising SEQ ID No. 17.
